SEQUENCE LISTING (46 sequences)

SEQ ID NO: 1 - RNA, 19 nt, Homo sapiens
uccgacuaccuuguggucu

SEQ ID NO: 2 - RNA, 19 nt, Homo sapiens
aaggaguugcagcgguuca

SEQ ID NO: 3 - RNA, 19 nt, Homo sapiens
cuguuucuaucgggcuuuc

SEQ ID NO: 4 - RNA, 19 nt, Homo sapiens
gcuuucggauucucccacu

SEQ ID NO: 5 - RNA, 19 nt, Homo sapiens
gcugugucugccaagagca

SEQ ID NO: 6 - RNA, 19 nt, Homo sapiens
cacguucauggaccugauu

SEQ ID NO: 7 - DNA, 2016 bp, Artificial Sequence
  Sequence encoding a CAR polypeptide
atggctttgccagtgacagctcttctccttccactggccctcctccttcacgccgctaggccagagcagaaacttatttcagaggaagacctggacattcaaatgacacaaactacttcttctctctccgcctcacttggtgaccgcgtcactattagttgccgcgctagtcaagatattagtaagtacctgaattggtatcaacaaaaacctgacgggactgtaaagctgcttatatatcatacttctaggctgcattctggagtaccttcacgatttagcggtagcggatccggcaccgactactccctcacaattagcaatctggagcaagaggacatagccacctacttctgccagcaagggaataccttgccatacactttcggtggtggaactaagctcgaaattactgggggtggaggcagtggcggaggggggtcaggtgggggaggttcagaagtcaaactccaggaatctggacctggactcgttgccccttcccaatcccttagtgttacatgcactgtatcaggtgtatccctccctgattacggtgtctcctggattcggcagcctcctcggaagggtctcgagtggttgggagtgatttgggggtctgaaactacttattataacagtgcccttaagagtagattgactataattaaggataacagtaagtcacaagtattcctcaaaatgaattccttgcaaacagacgatacagcaatatattactgcgcaaaacactactactatggcggtagttacgctatggactattggggtcaaggaacctctgtcacagtttctagcattgagttcatgtatcccccaccttacttggacaatgaaaggtctaatgggaccatcatacacattaaagagaaacacctgtgtcatactcagagttctccaaaattgttctgggccttggttgtcgttgccggcgtactgttctgttacggtctcttggttaccgtggcactttgtgttatctggactaattcccggcggaatcggggtggacagagcgattacatgaatatgaccccaagaagacctggactgaccaggaaaccatatcaaccctatgctcctgctcgggactttgctgcttaccgcccacgcgcaaagttttctaggagcgctgaaaccgctgccaacctccaagaccctaatcagctttacaatgaattgaacttgggacgccgggaggagtatgacgtccttgagaaaaagcgggctcgggatccagaaatgggcggaaagcaacagaggcgaagaaatccacaagagggggtctataacgctcttcagaaagataaaatggctgaggcatatagcgaaattgggaccaagggggagagaagaagaggcaagggacatgacgggctttaccagggtttgtctaccgcaacaaaagacacctatgatgctttgcacatgcaaacactggctcctagagccaccaacttctccctgctgaagcaggccggcgacgtggaggagaaccccggccccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtga

SEQ ID NO: 8 - protein, 671 aa, Artificial Sequence
  CAR polypeptide
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 9 - DNA, 22 bp, Artificial Sequence
  Synthetic oligonucleotide
cgtgaaaagatgacccagatca

SEQ ID NO: 10 - DNA, 20 bp, Artificial Sequence
  Synthetic oligonucleotide
cacagcctggatggctacgt

SEQ ID NO: 11 - DNA, 20 bp, Artificial Sequence
  Synthetic oligonucleotide
gtagcgactccgaaggtgtt

SEQ ID NO: 12 - DNA, 20 bp, Artificial Sequence
  Synthetic oligonucleotide
accagaggattctgcacagc

SEQ ID NO: 13 - DNA, 20 bp, Artificial Sequence
  Synthetic oligonucleotide
agcaccagccaagccatgta

SEQ ID NO: 14 - DNA, 21 bp, Artificial Sequence
  Synthetic oligonucleotide
cgtagggagaggtgctgtttt

SEQ ID NO: 15 - DNA, 20 bp, Artificial Sequence
  Synthetic oligonucleotide
gcagtcgtgtttgtcactcg

SEQ ID NO: 16 - DNA, 20 bp, Artificial Sequence
  Synthetic oligonucleotide
agagcaagcaatgacaggga

SEQ ID NO: 17 - DNA, 21 bp, Artificial Sequence
  Synthetic oligonucleotide
tctgtgcactgtgcatctctc

SEQ ID NO: 18 - DNA, 21 bp, Artificial Sequence
  Synthetic oligonucleotide
gacttggtgcatggaacactg

SEQ ID NO: 19 - DNA, 24 bp, Artificial Sequence
  Synthetic oligonucleotide
tgaatgaaccttccaagactcaga

SEQ ID NO: 20 - DNA, 22 bp, Artificial Sequence
  Synthetic oligonucleotide
ggttatggtcgatctttagctg

SEQ ID NO: 21 - DNA, 20 bp, Artificial Sequence
  Synthetic oligonucleotide
tgtcagcgtgcgacatggct

SEQ ID NO: 22 - DNA, 20 bp, Artificial Sequence
  Synthetic oligonucleotide
gagtgaagcggcggctggtg

SEQ ID NO: 23 - DNA, 20 bp, Artificial Sequence
  Synthetic oligonucleotide
ataccccctcgctctctgtt

SEQ ID NO: 24 - DNA, 20 bp, Artificial Sequence
  Synthetic oligonucleotide
acataggtccccatctgcct

SEQ ID NO: 25 - DNA, 20 bp, Artificial Sequence
  Synthetic oligonucleotide
gggatcatcttgctggtgaa

SEQ ID NO: 26 - DNA, 20 bp, Artificial Sequence
  Synthetic oligonucleotide
aggtccctgtcatgcttctg

SEQ ID NO: 27 - DNA, 20 bp, Artificial Sequence
  Synthetic oligonucleotide
agacatccgttccccctaca

SEQ ID NO: 28 - DNA, 20 bp, Artificial Sequence
  Synthetic oligonucleotide
gcagggtgctgacataccat

SEQ ID NO: 29 - DNA, 21 bp, Artificial Sequence
  Synthetic oligonucleotide
ccatggaaccgatcagtgtga

SEQ ID NO: 30 - DNA, 20 bp, Artificial Sequence
  Synthetic oligonucleotide
ttttcatcccggaagcaggg

SEQ ID NO: 31 - DNA, 22 bp, Artificial Sequence
  Synthetic oligonucleotide
ctcaggagcccacaacgagtgc

SEQ ID NO: 32 - DNA, 23 bp, Artificial Sequence
  Synthetic oligonucleotide
tctgggcttcttgcctcttgggt

SEQ ID NO: 33 - DNA, 21 bp, Artificial Sequence
  Synthetic oligonucleotide
cgggaagagctctggagaacc

SEQ ID NO: 34 - DNA, 22 bp, Artificial Sequence
  Synthetic oligonucleotide
gcattctctaacagtctgtgcc

SEQ ID NO: 35 - DNA, 20 bp, Artificial Sequence
  Synthetic oligonucleotide
gccagggaaccgcttatatg

SEQ ID NO: 36 - DNA, 23 bp, Artificial Sequence
  Synthetic oligonucleotide
gacgatcatctgggtcacattgt

SEQ ID NO: 37 - DNA, 20 bp, Artificial Sequence
  Synthetic oligonucleotide
tcatcacctacagcgacgag

SEQ ID NO: 38 - DNA, 20 bp, Artificial Sequence
  Synthetic oligonucleotide
gggtagaaggtggggatttc

SEQ ID NO: 39 - DNA, 22 bp, Artificial Sequence
  Synthetic oligonucleotide
gcctcttatgcttcaaacaaca

SEQ ID NO: 40 - DNA, 20 bp, Artificial Sequence
  Synthetic oligonucleotide
cagcagcattgccaaacagt

SEQ ID NO: 41 - DNA, 21 bp, Artificial Sequence
  Synthetic oligonucleotide
gaaacccctccctcttcagga

SEQ ID NO: 42 - DNA, 22 bp, Artificial Sequence
  Synthetic oligonucleotide
agggctgcacagataaaacttc

SEQ ID NO: 43 - DNA, 20 bp, Artificial Sequence
  Synthetic oligonucleotide
gatcgccggattggaacaga

SEQ ID NO: 44 - DNA, 20 bp, Artificial Sequence
  Synthetic oligonucleotide
ggtctagctgcttagcgtcc

SEQ ID NO: 45 - DNA, 20 bp, Artificial Sequence
  Synthetic oligonucleotide
gctgtgcttatgggcttctc

SEQ ID NO: 46 - DNA, 20 bp, Artificial Sequence
  Synthetic oligonucleotide
cacatacatgggcacaaagc

(19) United States
(12) Patent Application Publication
(10) Pub. No.: US 2022/0233594 A1
(43) Pub. Date: Jul. 28, 2022

(54) TARGETING OTUB1 IN IMMUNOTHERAPY

(71) Applicant: Board of Regents, The University ofTexas System, Austin, TX (US)

(72) Inventors: Shao-Cong SUN, Houston, TX (US); Xiaofei ZHOU, Houston, TX (US)

(73) Assignee: Board of Regents, The University of Texas System, Austin, TX (US)

(21) Appl. No.: 17/609,507

(22) PCT Filed: May 7, 2020
(86) PCT No.: PCT/US20/31816

§ 371 Date: Nov. 8, 2021

Related U.S. Application Data
(60) Provisional application No. 62/844,217, filed on May 7, 2019.

(51) Int. Cl. A61K 35/17; A61P 35/00; A61K 38/17; A61K 45/06; A61K 31/675; A61K 31/7076; A61P 37/04

(52) U.S. Cl. CPC A61K 35/17; A61P 35/00; A61K 38/1774; A61P 37/04; A61K 31/675; A61K 31/7076; A61K 45/06

(57) ABSTRACT

The present disclosure provides methods for generating Otub 1 deficient T cells and natural killer (NK) cells and compositions comprising engineered T cells expressing a reduced amount of Otub 1. Further provided are methods of treating cancer comprising administering the Otub 1 deficient T cells and/or NK cells to a subject in need thereof.

### REFERENCE TO RELATED APPLICATIONS

The present application claims the priority benefit of U.S. provisional application No. 62/844,217, filed May 7, 2019, the entire contents of which is incorporated herein by reference.

### STATEMENT REGARDING FEDERALLY SPONSORED RESEARCH

This invention was made with government support under Grant Nos. AI064639, AI057555, and GM084459 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on May 6, 2020, is named UTFC.P1462WO_ST25.txt and is 17.6 kilobytes in size.

### BACKGROUND

### 1. Field

The present invention relates generally to the fields of medicine and oncology. More particularly, it concerns T cells and NK cells having reduced levels of Otub1 protein and their use in treating cancer.

### 2. Description of Related Art

CD8 T cells and natural killer (NK) cells are major cytotoxic effector cells of the immune system responsible for destruction of pathogen-infected cells and cancer cells (Durgeau et al., 2018; Chiossone et al., 2018). CD8 T cells detect specific antigens via the T cell receptor (TCR), while NK cells are innate lymphocytes that use different receptors for sensing target cells. These effector cells also function in different phases of an immune response, with NK cells acting in the early phase of innate immunity and CD8 T cells acting in the late phase of adaptive immunity. NK cells also play an important role in regulating T cell responses (Crouse et al., 2015). Thus, CD8 T cells and NK cells are considered complementary cytotoxic effectors and have been actively explored for cancer immunotherapy (Rosenberg & Huang, 2018).

A common feature of CD8 T cells and NK cells is their dependence on the cytokine IL-15 for homeostasis (Surh & Sprent, 2008; Castillo & Schluns, 2012). IL-15 is a member of common gamma-chain (γc) family cytokines that functions through the IL-15 receptor (IL-15R) complex, composed of IL-15Rα, IL-15Rβ (also called IL-2Rβ or CD122), and γc (also called CD132). IL-15 induces signaling via a transpresentation mechanism, in which IL-15Ra binds to IL-15 and transpresents IL-15 to the IL-15R β/γ complex on responding cells (Castillo & Schluns, 2012). Under physiological conditions, IL-15 is specifically required for the homeostasis of CD8 T cells and NK cells that express high levels of IL-15R βγ heterodimer (Schluns et al., 2000; Schluns & Legrancois, 2003). Exogenously administered IL-15 can also promote activation of CD8 T cells and NK cells and, therefore, has been exploited as an adjuvant for cancer immunotherapies (Liu et al., 2002; Deshpande et al., 2013; Teague et al., 2006). However, the physiological function of IL-15 in regulating the activation of CD8 T cells and NK cells is poorly defined, and how the signal transduction from IL-15R is regulated is also elusive.

Ubiquitination has become a crucial mechanism that regulates diverse biological processes, including immune responses (Hu & Sun, 2016). Ubiquitination is a reversible reaction counter-regulated by ubiquitinating enzymes and deubiquitinases (DUBs) (Sun, 2008). In vitro studies identified an atypical DUB, Otub1, which can both directly cleave ubiquitin chains from target proteins and indirectly inhibit ubiquitination via blocking the function of specific ubiquitin-conjugating enzymes (E2s), including the K63-specific E2 Ubc13 (Juang et al., 2012; Nakada et al., 2010; Wang et al., 2009; Wiener et al., 2012). However, the in vivo physiological function of Otub1 has been poorly defined.

### SUMMARY

Otub1 (ubiquitin thioesterase) is a pivotal regulator of IL-15R signaling and homeostasis of CD8 T cells and NK cells. Otub1 controls IL-15-stimulated activation of AKT, a pivotal kinase for T cell activation, metabolism, and effector functions (Gubser et al., 2013; Kim & Suresh, 2013; Cammann et al., 2016). Otub1 controls the activation and function of CD8 T cells and NK cells in immune responses against infections and cancer.

In one embodiment, provided herein are ex vivo methods for producing CD8 T cells and/or natural killer (NK) cells modified to express a reduced level of Otub1 compared to unmodified CD8 T cells and/or NK cells comprising: (a) culturing a starting population of CD8 T cells and/or NK cells; (b) introducing a vector that inhibits the expression of Otub1; and (c) expanding the modified CD8 T cells and/or NK cells.

In some aspects, the vector encodes an Otub1 inhibitory RNA. In some aspects, the vector encodes an shRNA that inhibits Otub1 mRNA expression. In some aspects, the shRNA targets a sequence selected from the group consisting of CUGUUUCUAUCGGGCUUUC (SEQ ID NO: 3), GCUUUCGGAUUCUCCCACU (SEQ ID NO: 4), GCUGUGUCUGCCAAGAGCA (SEQ ID NO: 5), and CACGUUCAUGGACCUGAUU (SEQ ID NO: 6). In some aspects, the vector encodes an Otub1 inhibitor RNA comprising an shRNA that binds to the sequence of either SEQ ID NO: 1 or 2. In some aspects, the vector encodes a construct to modify the Otub1 gene, thereby preventing Otub1 expression. In some aspects, the vector is a lentiviral vector or retroviral vector. In some aspects, introducing comprises transduction, transfection, or electroporation. In some aspects, the modified CD8 T cells and/or NK cells are further modified to express a CAR and/or a TCR. In some aspects, the starting population of CD8 T cells and/or NK cells is obtained from a sample of autologous tumor infiltrating lymphocytes having antitumor activity, cord blood, peripheral blood, bone marrow, CD34⁺ cells, or induced pluripotent stem cells (iPSCs). In some aspects, the population of modified CD8 T cells and/or NK cells are GMP-compliant.

In one embodiment, provided herein are populations of modified CD8 T cells and/or NK cells produced according to the methods of any one of the present embodiments.

In one embodiment, provided herein are pharmaceutical compositions comprising the population of modified CD8 T cells and/or NK cells of any one of the present embodiments and a pharmaceutically acceptable carrier.

In one embodiment, provided herein are compositions comprising an effective amount of the modified CD8 T cells and/or NK cells of any one of the present embodiments for use in the treatment of a cancer in a subject.

In one embodiment, provided herein are uses of a composition comprising an effective amount of the modified CD8 T cells and/or NK cells of any one of the present embodiments for the treatment of a cancer in a subject.

In one embodiment, provided herein are methods of treating a cancer in a patient comprising administering an anti-tumor effective amount of modified CD8 T cells and/or NK cells of any one of the present embodiments to the subject.

In some aspects, the cancer is a solid cancer or a hematologic malignancy. In some aspects, the modified CD8 T cells and/or NK cells are autologous to the patient. In some aspects, the modified CD8 T cells and/or NK cells are derived from a sample of autologous tumor infiltrating lymphocytes having antitumor activity. In some aspects, the modified CD8 T cells and/or NK cells are allogeneic. In some aspects, the modified CD8 T cells and/or NK cells are HLA matched to the patient.

In some aspects, the modified CD8 T cells express a CAR polypeptide and/or a TCR polypeptide. In some aspects, the modified CAR and/or TCR has antigenic specificity for CD19, CD319/CS1, ROR1, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

In some aspects, the modified CD8 T cells and/or NK cells are administered to the subject intravenously, intraperitoneally, or intratumorally. In some aspects, the methods further comprise administering at least one additional therapeutic agent to the patient. In some aspects, the at least one additional therapeutic agent is selected from the group consisting of chemotherapy, radiotherapy, and immunotherapy. In some aspects, the at least one additional therapeutic agent is an immunotherapy, such as an immune checkpoint inhibitor. In some aspects, the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR). In some aspects, the immune checkpoint inhibitor inhibits PD-1 or CTLA-4.

In some aspects, the methods further comprise lymphodepletion of the subject prior to administration of the modified CD8 T cells and/or NK cells. In some aspects, lymphodepletion comprises administration of cyclophosphamide and/or fludarabine.

In some aspects, the methods increase the frequency of CD8 effector T cells in the patient's cancer. In some aspects, the methods increase the frequency of stage 4 mature NK cells in the patient's cancer. In some aspects, the methods overcome immune tolerance in the patient. In some aspects, the methods reduce CD8 T cell self-tolerance in the patient. In some aspects, the methods increase the number of tumor infiltrating CD8 T cells and NK cells in the patient's cancer.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-H. Otub1 regulates the homeostasis and activation of CD8 T cells. FIG. 1A, Flow cytometric analysis of naive (CD44^{lo}CD62L^{hi}) and memory (CD44^{hi}CD62L^{lo}) CD4 T cells and naive (CD44^{lo}) and memory (CD44^{hi}) CD8 T cells in the spleen of WT and Otub1-TKO (TKO) mice. Data are presented as a representative plot (upper) and summary graphs (lower) based on multiple mice (WT, n=13; TKO, n=8). FIGS. 1B&C, Flow cytometric analysis of intracellular IFN-γ, TNF and IL-2 in WT and Otub1-TKO splenic CD8 T cells (FIG. 1B) or CD4 T cells (FIG. 1C), stimulated for 4 h with PMA and ionomycin in the presence of monensin. Data are presented as representative plots (left) and summary graphs (right) based multiple mice (WT, n=5; TKO, n=5). FIGS. 1D&E, ELISA of the indicated cytokines in the culture supernatant of naive CD8 and CD4 T cells (FIG. 1D) or OT-I CD8 T cells (FIG. 1E) purified from the spleen of young (6 wk, n=4) WT and Otub1-TKO mice and stimulated for 66 h with plate-bound anti-CD3 (1 μg/ml) and anti-CD28 (1 μg/ml). FIGS. 1F-H, Liver bacteria titer (FIG. 1F) and flow cytometric analysis of IFN-γ-producing CD8 effector T cell frequency in OVA257-264-stimulated splenic T cells (FIGS. 1G&H) derived from WT and Otub1-TKO mice (FIG. 1G, WT: n=6; TKO: n=4) or WT OT-I and TKO OT-I mice (FIG. 1H, WT OT-I: n=6; TKO OT-I: n=5) infected with LM-OVA for 7 days. Data summarize three (FIGS. 1B-H) or five (FIG. 1A) independent experiments. Summary graphs are presented as mean±s.e.m. with P values being determined by two-tailed Student's t-test. *P<0.05, **P<0.01, ***P<0.0001. Numbers in quadrants indicate percentage of cells.

FIGS. 2A-H. Otub1 controls IL-15-mediated homeostatic responses and priming of CD8 T cells. FIGS. 2A-C, Schematic of experimental design (FIG. 2A), a representative plot (FIG. 2B), and summary graph (FIG. 2C) of flow cytometric analyses of memory (CD44^{hi}) and naive (CD44^{lo}) CD8 T cells from Il15ra^{+/+} or Il15ra^{-/-} recipient mice 7 days after adoptive transfer with carboxyfluorescein succinimidyl ester (CFSE)-labeled WT and Otub1-TKO naive CD8 T cells. The WT and Otub1-TKO CD8 T cells were detected as CFSE⁺ cells and distinguished based on CD45 congenic marker (WT: CD45.1⁺; TKO: CD45.2+). FIG. 2D, Cell proliferation assays (based on CFSE dilution) of WT and Otub1-TKO OT-I cells isolated from sublethally irradiated Il15ra^{+/+} or Il15ra^{-/-} recipient mice 8 days after adoptive transfer with a mixture (1:1 ratio, 12×10⁶ cells) of CFSE-labeled WT OT-I (CD45.1+CD45.2+) and Otub1-TKO OT-I (TKO OT-I; CD45.2+) cells. FIGS. 2E&F, ELISA (FIG. 2E) and intracellular IFN-γ flow cytometric analysis (FIG. 2F) of WT and Otub1-TKO OT-I cells isolated from Il15ra^{+/+} or Il15ra^{-/-} recipient mice 7 days after being adoptively transferred with a mixture (1:1 ratio, 6×10⁶ cells) of CFSE-labeled WT OT-I (CD45.1⁺CD45.2⁺) and Otub1-TKO OT-I (TKO OT-I; CD45.2⁺) cells (n=4 for Il15ra^{+/+} and Il15ra^{-/-} recipients in FIG. 2E). In FIG. 2E, the bars in each graph represent, from left to right, WT OT-I and Il15ra^{+/+} recipient, KO OT-I and Il15ra^{+/+} recipient, WT OT-I and Il15ra^{-/-} recipient, and KO OT-I and Il15ra^{-/-} recipient. In FIG. 2F, each column represents, from left to right, WT OT-I and Il15ra^{+/+} recipient, KO OT-I and Il15ra^{+/+} recipient, WT OT-I and Il15ra^{-/-} recipient, and KO OT-I and Il15ra^{-/-} recipient. FIG. 2G, Heatmap showing a list of effector/memory-related genes and stem memory T cell (Tscm) genes from RNA sequencing analysis of untreated WT and Otub1-TKO naive OT-I CD8 T cells freshly isolated from young mice (6 wk). FIG. 2H, qRT-PCR analysis of the indicated genes in WT and Otub1-TKO OT-I cells freshly isolated from Il15ra^{+/+} and Il15ra^{-/-} recipient mice 7 days after being adoptively transferred with a mixture (1:1 ratio, 6×10⁶ cells) of CFSE-labeled WT OT-I (CD45.1⁺CD45.2⁺) and TKO OT-I (CD45.2⁺) cells (WT recipients: n=4; Il15ra^{-/-} recipients: n=5). In FIG. 2H, each group of bars represents, from left to right, WT OT-I and Il15ra^{+/+} recipient, KO OT-I and Il15ra^{+/+} recipient, WT OT-I and Il15ra^{-/-} recipient, and KO OT-I and Il15ra^{-/-} recipient. Data are representative of one experiment (FIG. 2G) or summarize three (FIGS. 2B-F&H) independent experiments. Summary data are mean±s.e.m. with P values being determined by two-tailed Student's t-test. *P<0.05, **P<0.01, ***P<0.001, ****P<0.0001.

FIGS. 3A-H. Otub1 controls the maturation and activation of NK cells. FIGS. 3A&B, Schematic of experimental design for producing Otub1 tamoxifen-induced KO (iKO) and WT control mice (FIG. 3A) and immunoblot analysis of Otub1 in splenocytes of Otub1-iKO and WT mice (FIG. 3B). FIGS. 3C-E, Flow cytometric analysis of the frequency of naive (CD44^{lo}) and memory-like (CD44^{hi}) CD8 T cells (FIG. 3C), NK cells (FIG. 3D), and maturation stage subpopulations of NK cells (FIG. 3E, stage 2: CD11b^{lo} CD27^{hi}; stage 3: CD11b^{hi}CD27w; and stage 4: CD11b^{hi}CD27^{lo}). FIGS. 3F-H, Flow cytometric analysis of intracellular granzyme B (FIG. 3F) and CCL5 (FIGS. 3G&H) in WT or Otub1-iKO NK cells stimulated in vitro with IL-2 (5 ng/ml), IL-12 (10 ng/ml), and IL-18 (10 ng/ml) for the indicated time periods. The CCL5 results were presented as histogram (FIG. 3G) and dot plot (FIG. 3H). Data summarize two (FIGS. 3B-E) or three (FIGS. 3F-H) independent experiments. Summary data are mean±s.e.m. with P values being determined by two-tailed Student's t-test. *P<0.05, **P<0.01, ***P<0.001, ****P<0.0001.

FIGS. 4A-K Otub1 controls AKT axis of IL-15R signaling and is located to membrane compartment in an IL-15-dependent manner. FIGS. 4A-C, Immunoblot analyses of the indicated phosphorylated (P-) and total proteins in IL-15-stimulated CD8 T cells from 6-week old WT and Otub1-TKO OT-I mice (FIG. 4A), 15R-KIT cells transduced with either a control shRNA (sh-Ctrl) or two different Otub1-silencing shRNAs (Sh-Otub1) (FIG. 4B), or NK cells from tamoxifen-induced Otub1 KO (iKO) and WT control mice (FIG. 4C, NK cells were collected from 16 WT and 15 iKO mice). FIG. 4D, Immunoblot analyses of the indicated phosphorylated (P-) and total proteins in CD8 T cells from WT and Otub1-TKO OT-I mice (6 weeks old) stimulated with anti-CD3 plus anti-CD28. FIGS. 4E&F, Schematic of experimental design (FIG. 4E) and representative plots (FIG. 4F) of flow cytometric analyses of S473-phosphorylated AKT in WT and Otub1-TKO OT-I cells sorted from Il15ra^{+/+} and Il15ra^{-/-} recipients 7 days after being adoptively transferred with a mixture of CFSE-labeled WT OT-I and TKO OT-I CD8 T cells and stimulated in vitro with anti-CD3 plus anti-CD28 for 5 min. FIGS. 4G&H, Immunoblot analysis of the indicated proteins in membrane (Mem) and cytosol (Cyt) fractions or whole-cell lysates (whole-cell) of untreated CD4 T, CD8 T, and NK cells (FIG. 4G) or anti-CD3/anti-CD28-stimulated CD4 and CD8 T cells (FIG. 4H). FIGS. 4I&J, Schematic of experimental design (FIG. 4I) and immunoblot analysis (FIG. 4J) of Otub1 and the indicated loading controls in membrane (Mem) and cytosol (Cyt) fractions of WT OT-I CD8 T cells sorted from Il15ra^{+/+} or Il15ra^{-/-} recipients 7 days after adoptive transfer. FIG. 4K, Immunoblot analysis of Otub1, membrane protein IGF1RP, and cytoplasmic protein α-Tubulin in membrane (Mem) and cytoplasmic (Cyt) fractions of OT-I CD8 T cells sorted from WT OT-I mice injected (i.p.) with an IL-15 neutralizing antibody (200 mg/mouse) daily for three consecutive days. Data summarize two (FIGS. 4C,F,H,J,K), three (FIGS. 4B,D,G), or six (FIG. 4A) independent experiments.

FIGS. 5A-N. Otub1 inhibits K63 ubiquitination, PIP3-binding, and membrane translocation of AKT. FIG. 5A, Immunoblot analysis of AKT in membrane (Mem) and cytosol (Cyt) fractions of IL-15-stimulated 15R-KIT T cells transduced with either a control shRNA or two different Otub1 shRNAs. FIGS. 5B&C, Co-immunoprecipitation analysis of endogenous Otub1-AKT interaction in IL-15-stimulated 15R-KIT T cells (FIG. 5B) and primary OT-I CD8 T cells (FIG. 5C). FIG. 5D, AKT ubiquitination analyses in IL-15-stimulated 15R-KIT T cells stably expressing HA-ubiquitin.

FIG. 5E, AKT ubiquitination analysis in IL-15-stimulated Otub1-knockdown and control 15R-KIT T cells stably expressing HA-AKT. FIG. 5F, AKT ubiquitination analyses in HEK293T cells transiently transfected with HA-tagged WT, K63, or K48 ubiquitin in the presence (+) or absence (−) of the indicated expression vectors. Otub1 Mut harbors D88A/C91S mutations. FIGS. 5G&H, Ubiquitination analysis of WT and mutant forms of AKT in transiently transfected HEK293 cells (FIG. 5G) or IL-15-stimulated 15R-KIT T cells stably expressing the indicated HA-AKT WT and mutants (FIG. 5H). FIG. 5I, Immunoblot analysis of phosphorylated (P) and total AKT immunoprecipitated from IL-15-stimulated 15R-KIT T cells stably expressing AKT WT and mutants. FIGS. 5J&K, Schematic of ubiquitin K63 (UbK63)-AKT and UbK63-AKT K14R (FIG. 5J) and immunoblot analysis of their phosphorylation and total protein level immunoprecipitated from stably infected 15R-KIT T cells stimulated with IL-15 (FIG. 5K). FIG. 5L, Immunoblot analysis of ubiquitinated (upper) and total (lower) AKT or UbK63-AKT proteins immunoprecipitated from transiently transfected HEK293 cells. FIG. 5M, Immunoblot analysis of PIP3-bound (upper) and total (lower) HA-AKT proteins isolated by PIP3 bead-pull down (upper) and anti-HA IP (lower), respectively, from transiently transfected HEK293 cells. FIG. 5N, Immunoblot analysis of PIP3 bead-pull down (left) and anti-HA immunoprecipitated (right) AKT or UbK63-AKT proteins from transiently transfected HEK293 cells. Data summarize two (FIGS. 5A,K) or three (FIGS. 5B-I&L-N) independent experiments.

FIGS. 6A-J. Otub1 regulates gene expression and glycolytic metabolism in activated CD8 T cells. FIG. 6A, Heatmap showing a list of differentially expressed genes from RNA sequencing analyses of WT and Otub1-TKO OT-I CD8 T cells activated for 24 h with plate-coated anti-CD3 (1 μg/ml) plus soluble anti-CD28 (1 μg/ml). FIG. 6B, Immunoblot analysis of HK2 in WT or Otub1-TKO naive OT-I CD8 T cells that were either not treated (NT) or stimulated with anti-CD3 plus anti-CD28 for 24 h (activated). FIGS. 6C-F, Seahorse analysis of extracellular acidification rate (ECAR) under baseline (glucose injection) and stressed (oligomycin injection) conditions (FIGS. 6C,D) and Seahorse analysis of oxygen consumption rate (OCR) under baseline (no treatment) and stressed (FCCP injection) conditions (FIGS. 6E,F) in naive or anti-CD3/anti-CD28-activated (24 h) WT or Otub1-TKO naive OT-I CD8 T cells. Data are presented as a representative plot (FIGS. 6C,E) and summary graphs (FIGS. 6D,F). FIGS. 6G,H, Searhorse analysis of extracellular acidification rate (ECAR) in WT or Otub1-TKO naive OT-I CD8 T cells that were activated with anti-CD3 plus anti-CD28 for 24 h in the presence of an AKT inhibitor (AKTi, 3 μM) or solvent control DMSO. Data are presented as a representative plot (FIG. 6G) and summary graphs (FIG. 6H). FIGS. 6I,J, qRT-PCR analysis of Glut1 and Hk2 expression (FIG. 6I) and ELISA of the indicated cytokines in the culture supernatant (FIG. 6J) of WT or Otub1-TKO naive OT-I CD8 T cells that were either not treated (NT) or stimulated with anti-CD3 plus anti-CD28 in the presence of an AKT inhibitor (AKTi) or solvent control DMSO for the indicated time periods (FIG. 6I) or for 66 h (FIG. 6J). Data are representative of one (FIG. 6A) or summarize three (FIGS. 6B-J) independent experiments. Summary data are mean±s.e.m. with P values being determined by two-tailed Student's t-test. *P<0.05, **P<0.01, ***P<0.001, ****P<0.0001.

FIGS. 7A-J. Otub1 deficiency promotes CD8 T cell responses to a self-antigen. FIG. 7A, A representative image of 9-month old WT and Otub1-TKO Pmel1 mice. 100% TKO Pmel1 (n=21) and 0% WT Pmel1 (n=18) mice develop severe vitiligo (hair depigmentation). FIGS. 7B&C, Flow cytometric analysis of naive (CD44^{lo}) and memory (CD44^{hi}) T cell frequency (FIG. 7B) and CXCR3+ effector T cell frequency (FIG. 7C) of splenic CD8 T cells derived from WT Pmel1 and Otub1-TKO Pmel1 mice (WT, n=4; TKO, n=5). FIG. 7D, Flow cytometric analysis of IFN-γ-producing cells in WT and Otub1-TKO Pmel1 CD8 T cells stimulated for 6 h with GP10025-33 or control OVA257-264 peptide in the presence of monnesin (WT Pmel1, n=4, TKO Pmel1, n=5). Data are representative of one (FIG. 7A) or summarize three (FIGS. 7B-J) independent experiments. Summary data are mean±s.e.m. with P values being determined by two-tailed Student's t-test. *P<0.01, **P<0.001.

FIGS. 8A-O. Otub1 regulates anticancer immunity. FIGS. 8A-C, Tumor growth curve (FIG. 8A), day 18 tumor weight (FIG. 8B), and frequency of CD8 T cells and effector (IFN-γ⁺ and Granzyme B⁺) CD8 T cells (% of CD8 T cells) in the tumor and draining LN (dLN) (FIG. 8C) of WT or Otub1-TKO mice injected s.c. with 2×10⁵ B16-OVA cells (WT, n=6; TKO, n=5). FIG. 8D, Flow cytometric analysis of Glut1 expression in tumor-infiltrating CD8 T cells. FIGS. 8E-G, Schematic of experimental design (FIG. 8E), tumor growth curve (FIG. 8F), and Kaplan-Meier plot survival curve (FIG. 8G) of B6 mice that were inoculated with B16F10 melanoma cells and subsequently irradiated and injected with in vitro activated (with anti-CD3 plus anti-CD28 for 5 days) WT and Otub1-TKO Pmel1 T cells (6×10⁵). Control mice were inoculated with B16F10 cells without irradiation and Pmel1 T cell injection. Control, n=4; WT Pmel1, n=5; TKO Pmel1, n=5. In FIG. 8G, the lines represent, from left to right when read at 50% survival, Control, WT-Pmel1, and KO-Pmel1. FIGS. 8H-L, Schematic of experimental design (FIG. 8H), tumor growth curve (FIG. 8I), day 22 tumor weight (FIG. 8J), frequency of tumor-infiltrating immune cells (FIG. 8K), and frequency of tumor-infiltrating effector (IFN-γ⁺ and Granzyme B+) CD8 T cells (% of CD8 T cells) (FIG. 8L). FIGS. 8M-O, Tumor growth curve (FIG. 8M), day 21 tumor weight (FIG. 8N), and frequency of tumor-infiltrating immune cells (FIG. 8O) in WT and Otub1-iKO (iKO) mice inoculated with B16F10 melanoma cells and, where indicated, injected with NK cell- and CD8 T cell-depletion antibodies (anti-NK1.1 and anti-CD8a) as depicted in FIG. 14D). In FIG. 8O, each group of columns represents, from left to right, WT, iKO, iKO α-NK1.1, and iKO α-CD8. Data are representative of two (FIGS. 8A-G) or three (FIGS. 8H-0) independent experiments each with multiple biological replicates. Summary data are mean s.e.m. with P values being determined by two-way ANOVA with Bonferroni's post-test (FIGS. 8A,F,I,M), two-tailed Student's t-test (FIGS. 8B-D&J-L&N&O), or Log-Rank (FIG. 8G). *P<0.05, **P<0.01, ***P<0.001, ****P<0.0001.

FIGS. 9A-E. Otub1 deficiency does not influence the frequency of thymocyte and peripheral T cell populations. FIG. 9A, Schematic picture of Otub1 gene targeting using an FRT-LoxP vector. Targeted mice were crossed with FLP deleter (Rosa26-FLPe) mice to generate Otub1-floxed mice, which were further crossed with Cd4-Cre mice to generate T cell-conditional KO (TKO) mice. FIG. 9B, Genotyping PCR analysis of floxed and control mice using P1/P2 primer pair for WT allele and P3/P4 primer pair for flox allele. FIG. 9C, Immunoblot analysis of Otub1 using sorted T and B cells from WT or Otub1-TKO (KO) mice. FIG. 9D, Flow cytometric analysis of thymocytes from WT and Otub1-TKO (KO) mice (6 wk old), showing the percentage of CD4⁻CD8⁻ double negative, CD4⁺CD8⁺ double positive, and CD4⁺ and CD8⁺ single positive populations. A summary graph of total thymocyte cell number is shown. FIG. 9E, Flow cytometric analysis of frequency of CD4 and CD8 T cells in the splenocytes of WT and Otub1-TKO mice.

FIGS. 10A-E. Otub1 is dispensable for Treg cell generation and function. FIGS. 10A&B, Flow cytometric analysis of the frequency of Treg cells (Foxp3⁺CD25⁺) among CD4⁺ T cells in the thymus and spleen of age- and sex-matched WT and Otub1-TKO (KO) mice (6-8 weeks), presented as a representative plot (FIG. 10A) and summary graph based on multiple mice (FIG. 10B, each circle represents an individual mouse). FIG. 10C, Body weight loss of 6-week-old Rag1-KO mice following adoptive transfer with WT naïve CD45RB^{hi} CD4 T cells together with either PBS (CD45RB^{hi}+PBS) or sorted Treg cells derived from 6-week-old WT mice (CD45RB^{hi}+WT Treg) or Otub1-TKO mice (CD45RB^{hi}+KO Treg). FIG. 10D, Bone marrow cells (2×10⁶) from Otub1-TKO (KO, CD45.1-CD45.2⁺) and WT B6.SJL (WT, CD45.1⁺CD45.2-) mice were mixed in 1:1 ratio and adoptively transferred into γ-irradiated Rag1-KO mice. After 6 weeks, recipient mice were sacrificed for flow cytometric analyses of CD4 and CD8 T cells derived from WT and Otub1-KO (KO) bone marrows (left) and the naïve and memory populations based on CD44 and CD62L markers (naïve: CD44^{lo}CD62L^{hi}; memory: CD44^{hi}CD62L^{lo}) (right). FIG. 10E, Summary graphs of the naïve and memory T cell data from FIG. 10D based on four recipients of each group. *P<0.05 (two-tailed unpaired t test).

FIGS. 11A-E. IL-15 primes CD8 T cells for activation under the control of Otub1. FIG. 11A, ELISA of naïve CD8 T cells derived from WT, Otub1-TKO (TKO), WT/Il15ra^{-/-}, and Otub1-TKO/Il15ra^{-/-} mice, in vitro stimulated with anti-CD3 plus anti-CD28 for 66 h. FIG. 11B, Schematic of mixed T cell adoptive transfer and listeria infection. Il15ra^{+/+} or Il15ra^{-/-} mice were adoptively transferred with CFSE-labeled WT OT-I or Otub1-TKO OT-I naïve CD8 T cells mixed in 1:1 ratio (5×10⁶ cells each) and infected with ovalbumin-expressing recombinant Listeria monocytogenes (LM-OVA, 2×10⁴). Transferred OT-I cells were analyzed 7 days later. FIGS. 11C&D, Flow cytometric analysis of total population (FIG. 11C) or IFNg-producing effector frequency of WT and Otub1-TKO OT-I cells isolated from the LM-OVA-infected recipient mice shown in FIG. 11B, stimulated in vitro with OVA257-274 for 6 h (FIG. 11D). FIG. 11E, Scatterplot of significantly upregulated (pink, 6821 genes) and downregulated (blue, 1142 genes) genes in Otub1-TKO OT-I T cells relative to WT OT-I T cells. Some of the genes presented in the heatmap shown in FIG. 2G are indicated in green color. RNA sequencing was performed with RNA isolated from untreated naïve WT or Otub1-TKO OT-I CD8 T cells. NS, non-significant; *P<0.05; **P<0.01, two-tailed student's t-test.

FIGS. 12A-G. Otub1 negatively regulates AKT activation in CD8 T cells. FIGS. 12A-D, Immunoblot analysis of the indicated phosphorylated (P-) and total proteins in naïve OT-I CD8 T cells (FIGS. 12A&B), naïve CD8 T cells (FIG. 12C), or naïve CD4 T cells (FIG. 12D) stimulated with the indicated inducers. A panel of P-AKT T308 with 3 times more loading materials (3× loading) was included in FIG. 12A to visualize the weak AKT T308 phosphorylation stimulated by IL-15. FIG. 12E, Co-IP analysis of Otub1-AKT interaction in HEK293 cells transiently transfected with expression vectors encoding the indicated proteins. FIGS. 12F&G, Immunoblot analysis of the indicated phosphorylated (P-) and total proteins in IL-15-stimulated Otub1-deficient OT-I CD8 T cells (FIG. 12F) or Otub1-knockdown 15R-KIT T cells (FIG. 12G) infected with an empty retroviral vector or vectors encoding Otub1 wildtype (WT) or an inactive mutant (Mut, D88A/C91S).

FIG. 13. Otub1 controls gene expression in CD8 T cells. Scatterplot of significantly upregulated (pink, 1254) and downregulated (blue, 297) genes in Otub1-TKO (KO) OT-I T cells relative to WT OT-I T cells stimulated with anti-CD3 plus anti-CD28 for 24 h and analyzed by RNA sequencing. Some of the genes presented in the heatmap of FIG. 6A are indicated in green color.

FIGS. 14A-E. Otub1 deletion promotes antitumor immunity via CD8 T cells and NK cells. FIG. 14A, Schematic of experimental procedure, in which the indicated mice were injected with tamoxifen daily for 4 consecutive times starting from day 14 before tumor cell inoculation and one more time on day 7 after tumor inoculation for generating WT or Otub1 induced KO (iKO) MC38-bearing mice. FIG. 14B, Tumor burden of WT and Otub1-iKO mice, presented as tumor grow curve (left) and day 19 tumor weight (right). FIG. 14C, Summary graph of flow cytometric analysis of tumor-infiltrating immune cells in WT and Otub1-iKO mice. FIG. 14D, Schematic of experimental procedure, in which the indicated mice were injected with tamoxifen daily for 4 consecutive times starting from day 14 before tumor cell inoculation and one more time on day 7 after tumor inoculation for generating WT or Otub1 induced KO (iKO) B16F10-bearing mice. Some of the tumor-bearing mice were also injected i.p with anti-NK1.1 and anti-CD8a for depletion of NK cells and CD8 T cells, respectively. (FIG. 14E) Flow cytometric analysis of NK cells and CD8 T cells showing the efficiency of antibody-mediated depletion. P values are determined by two-way ANOVA with Bonferroni's post-test (FIG. 14B) or two-tailed student's t-test (FIG. 14C).

FIGS. 15A-C. Otub1 expression level is inversely associated with patient survival and effector T cell signature gene expression in skin cutaneous melanoma. FIG. 15A, Heatmap illustrating the expression of major CD8 effector T cell signature genes (rows) across the 458 skin cutaneous melanoma patients (columns). The color scale of the heatmap indicates relative gene expression. FIG. 15B, mRNA level of CD8 T cell signature genes in Otub1 low and high group. ****P<0.0001, two-tailed student's t-test. FIG. 15C, Kaplan-Meier plot comparing survival for the two broad clusters of patients identified in hierarchical clustering analysis. (p<0.0001, Log-Rank test). The top line represents Otub1 Low; the bottom line represents Otub1 High.

FIG. 16. Live immune cell populations were gated on the FSC-A and SSC-A, and single cells were gated basing on FSC-A and FAS-H. The subpopulations of the indicated immune cells were gated basing on specific surface markers as indicated in the individual panels.

FIGS. 17A-D. Generation of B16F10-hCD19 cell clone and anti-hCD19 CAR T cells. FIG. 17A, Flow cytometric analysis of CD19 in B16F10 cells transduced with a retroviral vector encoding human CD19 (hCD19). FIG. 17B, CAR construction with CD8α signal peptide, Myc epitope-Tag, anti-human CD19 scFv, mouse CD28, mouse CD3z signaling domain, the P2A self-cleaving peptide and the mouse Thy1.1 reporter. FIG. 17C, workflow of generating anti-hCD19 CAR T cells. FIG. 17D, Flow cytometric analysis of CAR expression in anti-hCD19 CAR-transduced murine CD8 T cells based on surface expression of Myc epitope tag and Thy1.1. Mock-transduced CD8+ T cells were used as controls.

FIGS. 18A-D. Genetic ablation of Otub1 promotes the activity of CAR T cells against B16 melanoma. FIG. 18A, Schematic of experimental design. B6 mice were inoculated with B16F10-hCD19 melanoma cells and, on day 7, adoptively transferred with anti-hCD19 CAR-transduced mouse CD8 T cells. FIGS. 18B,C, Tumor growth curve presented as a summary graph based on the indicated numbers of mice (FIG. 18B) and as curves of individual mice (FIG. 18C). In FIG. 18B, the lines represent, from top to bottom when read at 30 days after tumor injection, PBS, WT-CarT, and KO-CarT. FIG. 18D, Kaplan-Meier survival plot. Summary data are shown as mean±SEM with P values being determined by two-way ANOVA with Bonferroni correlation (FIG. 18A) or log-rank test (FIG. 18C).

FIGS. 19A-C. CAR T cell therapy using OT-I T cell model. B6 mice were inoculated with B16F10-hCD19 melanoma cells and, on day 7, adoptively transferred with anti-hCD19 CAR-transduced mouse OT-I CD8 T cells. The treated mice were monitored for tumor growth (FIGS. 19A and B) and survival (FIG. 19C) as described in the legend of FIG. 2. Summary data are shown as mean±SEM with P values being determined by two-way ANOVA with Bonferroni correlation (FIG. 19A) or log-rank test (FIG. 19C).

FIGS. 20A-E. ShRNA-mediated Otub1 knockdown increases the antitumor activity of CAR T cells. FIG. 20A, Immunoblot analysis of endogenous Otub1 in murine EL4 thymoma cells transduced with an Otub1-specific shRNA (F9) or a non-silencing (NS) control shRNA. FIG. 20B, Workflow for generating control or Otub1-knockdown anti-hCD19 CAR-transduced OT-I CD8 T cells. FIGS. 20C-E, Tumor growth summary curves based on multiple mice (FIG. 20C), tumor growth curves based on individual mice (FIG. 20D), and Kaplan-Meier survival plot (FIG. 20E) of B16F10-hCD19-bearing mice treated with control (NS) and Otub1 knockdown (F9) CAR T cells. Summary data are shown as mean±SEM with P values being determined by two-way ANOVA with Bonferroni correlation (FIG. 20C) or log-rank test (FIG. 20E).

FIGS. 21A-D. Genetic ablation of Otub1 increases the antitumor function of CAR NK cells. FIG. 21A, Workflow for generating anti-hCD19 CAR NK cells and adoptive transfer into tumor-bearing mice. FIGS. 21B-D, Tumor growth summary curves based on multiple mice (FIG. 21B), tumor growth curves based on individual mice (FIG. 21C), and Kaplan-Meier survival plot (FIG. 21D) of B16F10-hCD19-bearing mice treated with wildtype (WT) or Otub1-TKO (KO) CAR NK cells. Summary data are shown as mean±SEM with P values being determined by two-way ANOVA with Bonferroni correlation (FIG. 21B) or log-rank test (FIG. 21D).

FIGS. 22A-B. Generation and characterization of shRNAs targeting human Otub1. FIG. 22A, Sequences of four new human Otub1 shRNAs (H1-H4), as well as two commercially available human Otub1 shRNAs (#2 and #4), which were cloned into the pGIPZ lentiviral vector. Nucleotide numbers are based on the hOtub1 cDNA sequence. FIG. 22B, Immunoblot analysis of Otub1 and the loading control HSP60 in human 293T cells transduced with pGIPZ lentiviral vectors encoding a non-silencing (NS) control shRNA or the indicated Otub1 shRNAs, showing high knockdown efficiency of H2 and H3.

### DETAILED DESCRIPTION

CD8 T cells and natural killer (NK) cells, central cellular components of immune responses against pathogens and cancer, rely on IL-15 for homeostasis. IL-15 mediates homeostatic priming of CD8 T cells for antigen-stimulated activation, which is controlled by a deubiquitinase, Otub1. IL-15 mediates membrane recruitment of Otub1, which inhibits ubiquitin-dependent activation of AKT, a pivotal kinase for T cell activation and metabolism. Otub1 deficiency in mice causes aberrant responses of CD8 T cells to IL-15, rendering naive CD8 T cells hyper-sensitive to antigen stimulation characterized by enhanced metabolic reprograming and effector functions. Otub1 also controls the maturation and activation of NK cells. Otub1 controls the activation of CD8 T cells and NK cells by functioning as a checkpoint of IL-15-mediated priming. Consistently, Otub1 deletion profoundly enhances anticancer immunity through unleashing the activity of CD8 T cells and NK cells.

Chimeric antigen receptor (CAR)-transduced T cells targeting tumor-associated antigens have shown promise in the treatment of B cell malignancies; however, CAR T cell therapy is less effective against solid tumors because of tumor-infiltrated T cell exhaustion. While extensive effort has been made to modify CAR signaling motifs, much less is known about how to target intracellular factors for improving the efficacy of CAR T cell therapy. Using a human CD19 CAR T cell system, provided herein is pre-clinical evidence that Otub1 knockout or knockdown profoundly boosts the function of CAR T cells against hCD19-transduced solid tumors. Targeting Otub1 also enhances the function of CAR NK cells.

### I. ASPECTS OF THE PRESENT EMBODIMENTS

The results presented here suggest a ubiquitin-dependent mechanism that regulates IL-15R signaling and the IL-15-dependent homeostasis of CD8 T cells and NK cells and establish the DUB Otub1 as a crucial regulator. Otub1 controls IL-15-stimulated ubiquitination and activation of AKT, a kinase mediating the activation and metabolic reprograming of CD8 T cells. Despite the abundant expression of Otub1 in CD4 T cells, the Otub1 deficiency had no effect on the homeostasis of CD4 T cells. This cell type-specific function of Otub1 is explained by its role in regulating IL-15R signaling, which is specifically required for the homeostasis of CD8 T cells and NK cells (Schluns et al., 2000; Schluns & Lefrancois, 2003; Guillerey et al., 2016).

These data suggest that homeostatic exposure of CD8 T cells to IL-15 serves as a crucial priming step for antigen-specific CD8 T cell activation, which is controlled by Otub1. T cell-specific deletion of Otub1 rendered CD8 T cells hyper-responsive to bacterial infections in vivo and to activation by TCR-CD28 signals in vitro. This phenotype was due to aberrant priming of the naive CD8 T cells by IL-15, since it was not detected in IL-15Ra-deficient mice. In CD8 T cells and NK cells, Otub1 is located to the membrane compartment. The membrane localization of Otub1 was dependent on IL-15 signaling, thus implicating Otub1 as a checkpoint of IL-15-mediated CD8 T cell priming. Since AKT activation occurs in various membrane compartments (Jethwa et al., 2015), these findings suggest that the membrane localization of Otub1 may facilitate its role in regulating AKT activation.

Otub1 regulates different aspects of CD8 T cell activation and function. Otub1 deficiency sensitized CD8 T cells for activation by both TCR-CD28 stimuli and listeria infections and promoted generation of antigen-specific effector cells. The crucial role of Otub1 in regulating CD8 T cell responses was also revealed by the development of vitiligo in Otub1-TKO Pmel1 mice, which was due to aberrant CD8 T cell activation by the melanocyte self-antigen gp100. Another important function of Otub1 was to regulate the metabolic reprograming of activated CD8 T cells, an essential mechanism for supporting proliferation, effector cell generation and function (Pearce et al., 2013). This function of Otub1 is in line with its role in AKT regulation, since AKT is a master kinase mediating the activation, metabolism, and effector functions of CD8 T cells (Gubser et al., 2013; Kim & Suresh, 2013; Cammann et al., 2016).

Inducible deletion of Otub1 in adult mice greatly promoted tumor rejection, associated with increased tumor-infiltration with various immune cells, including CD8 T cells, NK cells, as well as CD4 T cells and cDC1 cells. Depletion of either NK cells or CD8 T cells impaired the anticancer immunity, erasing the differences between the WT and Otub1-iKO mice in tumor rejection. Antibody-mediated cell depletion studies revealed a crucial role for NK cells in mediating the recruitment of CD4 T cells and cDC1 cells in the Otub1-iKO mice. In an adoptive T cell therapy model, Otub1 deletion also profoundly enhanced the tumor-rejection activity of CD8 effector T cells, which was consistent with the role of Otub1 in regulating the metabolism and effector molecule expression of activated CD8 T cells. These findings implicate Otub1 as a potential drug target for cancer immunotherapy.

The role of ubiquitination in regulating IL-15R signaling has been poorly defined. The present results demonstrated Otub1 as a DUB specifically regulating AKT axis of IL-15R signaling. A central step in AKT activation is its recruitment to the plasma membrane, where it is activated via S473 phosphorylation by mTORC2 and T308 phosphorylation by PDK1 (Mishra et al., 2014). The membrane recruitment of AKT involves its binding, via N-terminal PH domain, to the membrane phospho-lipid PIP3. These data suggest that Otub1-mediated AKT deubiquitination attenuates its binding to PIP3. Notably, the ubiquitination site, K14, of AKT is located in its PH domain. It is thought that inactive AKT exists in a closed conformation due to intramolecular interaction between its N-terminal PH domain and C-terminal kinase domain (Calleja et al., 2009). Thus, ubiquitination of AKT in its PH domain may interfere with the intramolecular interaction, thereby facilitating the exposure of PH domain for PIP3 binding.

### II. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “haplotyping or tissue typing” refers to a method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are six genetic loci important in transplantation, designated HLA-A, HLA-B, HLA-C, and HLA-DR, HLA-DP and HLA-DQ. At each locus, there can be any of several different alleles.

A widely used method for haplotyping uses the polymerase chain reaction (PCR) to compare the DNA of the subject, with known segments of the genes encoding MHC antigens. The variability of these regions of the genes determines the tissue type or haplotype of the subject. Serologic methods are also used to detect serologically defined antigens on the surfaces of cells. HLA-A, -B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The pattern of cells destroyed by lysis indicates the degree of histologic incompatibility. If, for example, the lymphocytes from a person being tested for HLA-A3 are destroyed in a well containing antisera for HLA-A3, the test is positive for this antigen group.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, and dendritic cells, or molecules, naturally occurring or synthetic capable of presenting antigen, such as purified MHC Class I molecules complexed to 02-microglobulin.

### III. ENGINEERED CD8 T CELLS AND NK CELLS

The present disclosure provides methods for producing engineered CD8 T cells or NK cells that have altered expression of certain genes, such as Otub1. These engineered CD8 T cells and NK cells are contemplated for use in adoptive immunotherapy, which involves the transfer of autologous or allogeneic antigen-specific T cells generated ex vivo. The engineered CD8 T cells and NK cells may be further modified to express an antigen-specific receptor on their surface. Novel specificities in T cells have been successfully generated through the genetic transfer of transgenic T cell receptors or chimeric antigen receptors (CARs). CARs have successfully allowed T cells to be redirected against antigens expressed at the surface of tumor cells from various malignancies including lymphomas and solid tumors.

A. CD8 T Cell Preparation

The CD8 T cells may be derived from the blood, bone marrow, lymph, lymphoid organs, or tumor biopsies. In some aspects, the cells are human cells. The cells may be primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD8⁺ selection step is used to separate CD4⁺ helper and CD8⁺ cytotoxic T cells. Such CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of engineered T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T cell receptor stimulation in the presence of feeder lymphocytes and either interleukin-2 (IL-2) or interleukin-15 (IL-15). The non-specific T-cell receptor stimulus can include around 30 ng/ml of OKT3, a mouse monoclonal antibody for human anti-CD3 (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A1 (HLA-A1) binding peptide, in the presence of a T-cell growth factor, such as 300 IU/ml IL-2 or IL-15. The in vitro-induced T-cells are rapidly expanded by re-stimulation with the same antigen(s) of the cancer pulsed onto HLA-A1-expressing antigen-presenting cells. Alternatively, the T-cells can be re-stimulated with irradiated, autologous lymphocytes or with irradiated HLA-A1+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

B. NK Cell Preparation

The method may comprise obtaining a starting population of cells from cord blood, peripheral blood, bone marrow, CD34⁺ cells, or iPSCs, particularly from cord blood. The starting cell population may then be subjected to a Ficoll-Paque density gradient to obtain mononuclear cells (MNCs).

The MNCs can then be depleted of CD3, CD14, and/or CD19 cells for negative selection of NK cells or may be positively selected for NK cells by CD56 and/or CD16 selection. The selected NK cells may be characterized to determine the percentage of CD56⁺/CD3⁻ cells. The NK cells may then be incubated with APCs and cytokines, such as IL-2, IL-21, and IL-18 followed by Otub1 knock-down. The engineered NK cells can be further expanded in the presence of irradiated APCs and cytokines, such as IL-2 and IL-15.

The NK cells may be expanded in the presence of APCs, particularly irradiated APCs, such as UAPCs. The expansion may be for about 2-30 days or longer, such as 3-20 days, particularly 12-16 days, such as 12, 13, 14, 15, 16, 17, 18, or 19 days, specifically about 14 days. The NK cells and APCs may be present at a ratio of about 3:1-1:3, such as 2:1, 1:1, 1:2, specifically about 1:2. The expansion culture may further comprise cytokines to promote expansion, such as IL-2, IL-21, and/or IL-18. The cytokines may be present at a concentration of about 10-500 U/mL, such as 100-300 U/mL, particularly about 200 U/mL. The cytokines may be replenished in the expansion culture, such as every 2-3 days. The APCs may be added to the culture at least a second time, such as after CAR transduction.

Following expansion the immune cells may be immediately infused or may be stored, such as by cryopreservation. In certain aspects, the cells may be propagated for days, weeks, or months ex vivo as a bulk population within about 1, 2, 3, 4, 5 days.

Expanded NK cells can secrete type I cytokines, such as interferon-γ, tumor necrosis factor-α, and granulocyte-macrophage colony-stimulating factor (GM-CSF), which activate both innate and adaptive immune cells, as well as other cytokines and chemokines. The measurement of these cytokines can be used to determine the activation status of NK cells. In addition, other methods known in the art for determination of NK cell activation may be used for characterization of the NK cells of the present disclosure.

C. Modification of Gene Expression

In some embodiments, the immune cells of the present disclosure are modified to have altered expression of certain genes, such as Otub1. In some embodiments, the immune cells may be modified to express a decreased level of Otub1. In some embodiments, the immune cells may be modified such that the Otub1 gene is knocked out. The Otub1-KO immune cells may be administered to a cancer patient as part of a therapeutic regime. This approach may be used alone or in combination with other checkpoint inhibitors to improve anti-tumor activity.

In some embodiments, the altered gene expression is carried out by effecting a disruption in the gene, such as a knock-out, insertion, missense or frameshift mutation, such as biallelic frameshift mutation, and/or deletion of all or part of the gene, e.g., one or more exon or portion therefore. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, gene alteration is achieved using antisense techniques, such as by RNA interference (RNAi), small interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

In some embodiments, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

D. Genetically Engineered Antigen Receptors

The CD8 T cells and/or NK cells of the present disclosure can be genetically engineered to express antigen receptors such as engineered TCRs and/or CARs. For example, the CD8 T cells and NK cells are modified to express a TCR having antigenic specificity for a cancer antigen. Multiple CARs and/or TCRs, such as to different antigens, may be added to the CD8 T cells and NK cells.

1. Chimeric Antigen Receptors

Chimeric antigen receptor molecules are recombinant fusion protein and are distinguished by their ability to both bind antigen and transduce activation signals via immunoreceptor tyrosine-based activation motifs (ITAMs) present in their cytoplasmic tails. Receptor constructs utilizing an antigen-binding moiety (for example, generated from single chain antibodies (scFv) afford the additional advantage of being “universal” in that they bind native antigen on the target cell surface in an HLA-independent fashion.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

Embodiments of the CARs described herein include nucleic acids encoding an antigen-specific chimeric antigen receptor (CAR) polypeptide, including a comprising an intracellular signaling domain, a transmembrane domain, and an extracellular domain comprising one or more signaling motifs. In certain embodiments, the CAR may recognize an epitope comprised of the shared space between one or more antigens. In some embodiments, the chimeric antigen receptor comprises: a) an intracellular signaling domain, b) a transmembrane domain, and c) an extracellular domain comprising an antigen binding domain. Optionally, a CAR can comprise a hinge domain positioned between the transmembrane domain and the antigen binding domain. In certain aspects, a CAR of the embodiments further comprises a signal peptide that directs expression of the CAR to the cell surface. For example, in some aspects, a CAR can comprise a signal peptide from GM-CSF.

In certain embodiments, the CAR can also be co-expressed with a membrane-bound cytokine to improve persistence when there is a low amount of tumor-associated antigen. For example, CAR can be co-expressed with membrane-bound IL-15.

Depending on the arrangement of the domains of the CAR and the specific sequences used in the domains, immune effector cells expressing the CAR may have different levels activity against target cells. In some aspects, different CAR sequences may be introduced into immune effector cells to generate engineered cells, the engineered cells selected for elevated SRC and the selected cells tested for activity to identify the CAR constructs predicted to have the greatest therapeutic efficacy.

a. Antigen Binding Domain

In certain embodiments, an antigen binding domain can comprise complementary determining regions of a monoclonal antibody, variable regions of a monoclonal antibody, and/or antigen binding fragments thereof. In another embodiment, that specificity is derived from a peptide (e.g., cytokine) that binds to a receptor. A “complementarity determining region (CDR)” is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen—each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions.

It is contemplated that the CAR nucleic acids, in particular the scFv sequences are human genes to enhance cellular immunotherapy for human patients. In a specific embodiment, there is provided a full length CAR cDNA or coding region. The antigen binding regions or domains can comprise a fragment of the VH and VL chains of a single-chain variable fragment (scFv) derived from a particular mouse, or human or humanized monoclonal antibody. The fragment can also be any number of different antigen binding domains of an antigen-specific antibody. In a more specific embodiment, the fragment is an antigen-specific scFv encoded by a sequence that is optimized for human codon usage for expression in human cells. In certain aspects, VH and VL domains of a CAR are separated by a linker sequence, such as a Whitlow linker. CAR constructs that may be modified or used according to the embodiments are also provided in International (PCT) Patent Publication No. WO/2015/123642, incorporated herein by reference.

As previously described, the prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to an antigen of interest, such as tumor associated antigen. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, it is shown that a CAR may be engineered using VH and VL chains derived from different mAbs to generate a panel of CAR+ T cells. The antigen binding domain of a CAR can contain any combination of the LCDR1-3 sequences of a first antibody and the HCDR1-3 sequences of a second antibody.

b. Hinge Domain

In certain aspects, a CAR polypeptide of the embodiments can include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-gene modified T cells. In some aspects, the hinge domain comprises a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor. In certain aspects, the hinge domain (and/or the CAR) does not comprise a wild type human IgG4 CH2 and CH3 sequence.

In some cases the CAR hinge domain could be derived from human immunoglobulin (Ig) constant region or a portion thereof including the Ig hinge, or from human CD8 α transmembrane domain and CD8a-hinge region. In one aspect, the CAR hinge domain can comprise a hinge-CH2-CH3 region of antibody isotype IgG₄. In some aspects, point mutations could be introduced in antibody heavy chain CH₂ domain to reduce glycosylation and non-specific Fc gamma receptor binding of CAR-T cells or any other CAR-modified cells.

In certain aspects, a CAR hinge domain of the embodiments comprises an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. In some aspects, a CAR hinge domain comprises an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. In further aspects, a CAR hinge domain can comprise an IgG4-Fc domain having an amino acid substitution at position L235 for an amino acid that is hydrophilic, such as R, H, K, D, E, S, T, N or Q or that has similar properties to an “E” such as D. In certain aspects, a CAR hinge domain can comprise an IgG4-Fc domain having an amino acid substitution at position N297 for an amino acid that has similar properties to a “Q” such as S or T.

In certain specific aspects, the hinge domain comprises a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain or an engineered hinge domain.

c. Transmembrane Domain

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3ζ domain, or a cysteine mutated human CD3ζ domain, or other transmembrane domains from other human transmembrane signaling proteins, such as CD16 and CD8 and erythropoietin receptor. In some aspects, for example, the transmembrane domain comprises a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions of particular use in this invention may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

d. Intracellular Signaling Domain

The intracellular signaling domain of the chimeric antigen receptor of the embodiments is responsible for activation of at least one of the normal effector functions of the immune cell engineered to express a chimeric antigen receptor. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. In some aspects, the intracellular signaling domain is derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. In a preferred embodiment, the human CD3ζ intracellular domain is used as the intracellular signaling domain for a CAR of the embodiments.

In specific embodiments, intracellular receptor signaling domains in the CAR include those of the T cell antigen receptor complex, such as the (chain of CD3, also Fcγ RIII costimulatory signaling domains, CD28, CD27, DAP10, CD137, OX40, CD2, alone or in a series with CD3ζ, for example. In specific embodiments, the intracellular domain (which may be referred to as the cytoplasmic domain) comprises part or all of one or more of TCRζ chain, CD28, CD27, OX40/CD134, 4-1BB/CD137, FcεFRIγ, ICOS/CD278, IL-2Rβ/CD122, IL-2Rα/CD132, DAP10, DAP12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example the CD28 and 4-1BB can be combined in a CAR construct.

In some embodiments, the CAR comprises additional other costimulatory domains. Other costimulatory domains can include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

e. Suicide Genes

The CAR of the immune cells of the present disclosure may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

2. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable R chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the 3-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest,” US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and R (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). In some embodiments, phage display is used to isolate TCRs against a target antigen. In some embodiments, the TCR or antigen-binding portion thereof can be synthetically generated from knowledge of the sequence of the TCR.

3. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

4. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens. Tumor-associated antigens may be derived from prostate, breast, colorectal, lung, pancreatic, renal, mesothelioma, ovarian, or melanoma cancers. Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins. Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®. Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria. Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A Streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

5. Methods of Delivery

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

E. Bioreactor

The engineered T cells and/or NK cells may be expanded in a functionally closed system, such as a bioreactor. Expansion may be performed in a gas-permeable bioreactor, such as G-Rex cell culture device. The bioreactor may support between 1×10⁹ and 3×10⁹ total cells in an average 450 mL volume.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel).

Hollow fiber bioreactors can be used to enhance the mass transfer during culture. A Hollow fiber bioreactor is a 3D cell culturing system based on hollow fibers, which are small, semi-permeable capillary membranes arranged in parallel array with a typical molecular weight cut-off (MWCO) range of 10-30 kDa. These hollow fiber membranes are often bundled and housed within tubular polycarbonate shells to create hollow fiber bioreactor cartridges. Within the cartridges, which are also fitted with inlet and outlet ports, are two compartments: the intracapillary (IC) space within the hollow fibers, and the extracapillary (EC) space surrounding the hollow fibers.

Thus, for the present disclosure, the bioreactor may be a hollow fiber bioreactor. Hollow fiber bioreactors may have the cells embedded within the lumen of the fibers, with the medium perfusing the extra-lumenal space or, alternatively, may provide gas and medium perfusion through the hollow fibers, with the cells growing within the extralumenal space.

The hollow fibers should be suitable for the delivery of nutrients and removal of waste in the bioreactor. The hollow fibers may be any shape, for example, they may be round and tubular or in the form of concentric rings. The hollow fibers may be made up of a resorbable or non-resorbable membrane. For example, suitable components of the hollow fibers include polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, and mixtures thereof.

The bioreactor may be primed prior to seeding of the cells. The priming may comprise flushing with a buffer, such as PBS. The priming may also comprise coating the bioreactor with an extracellular matrix protein, such as fibronectin. The bioreactor may then be washed with media, such as alpha MEM.

In specific embodiments, the present methods use a GRex bioreactor. The base of the GRex flask is a gas permeable membrane on which cells reside. Hence, cells are in a highly oxygenated environment, allowing them to be grown to high densities. The system scales up easily and requires less frequent culture manipulations. GRex flasks are compatible with standard tissue culture incubators and cellular laboratory equipment, reducing the specialized equipment and capital investment required to initiate an ACT program.

The cells may be seeded in the bioreactor at a density of about 100-1,000 cells/cm², such as about 150 cells/cm², about 200 cells/cm², about 250 cells/cm², about 300 cells/cm², such as about 350 cells/cm², such as about 400 cells/cm², such as about 450 cells/cm², such as about 500 cells/cm², such as about 550 cells/cm², such as about 600 cells/cm², such as about 650 cells/cm², such as about 700 cells/cm², such as about 750 cells/cm², such as about 800 cells/cm², such as about 850 cells/cm², such as about 900 cells/cm², such as about 950 cells/cm², or about 1000 cells/cm². Particularly, the cells may be seeded at a cell density of about 400-500 cells/cm², such as about 450 cells/cm².

The total number of cells seeded in the bioreactor may be about 1.0×10⁶ to about 1.0×10⁸ cells, such as about 1.0×10⁶ to 5.0×10⁶, 5.0×10⁶ to 1.0×10⁷, 1.0×10⁷ to 5.0×10⁷, 5.0×10⁷ to 1.0×10⁸ cells. In particular aspects, the total number of cells seeded in the bioreactor are about 1.0×10⁷ to about 3.0×10⁷, such as about 2.0×10⁷ cells.

The cells may be seeded in any suitable cell culture media, many of which are commercially available. Exemplary media include DMEM, RPMI, MEM, Media 199, HAMS and the like. In one embodiment, the media is alpha MEM media, particularly alpha MEM supplemented with L-glutamine. The media may be supplemented with one or more of the following: growth factors, cytokines, hormones, or B27, antibiotics, vitamins and/or small molecule drugs. Particularly, the media may be serum-free.

In some embodiments the cells may be incubated at room temperature. The incubator may be humidified and have an atmosphere that is about 5% CO₂ and about 1% O₂. In some embodiments, the CO₂ concentration may range from about 1-20%, 2-10%, or 3-5%. In some embodiments, the O₂ concentration may range from about 1-20%, 2-10%, or 3-5%.

### IV. METHODS OF TREATMENT

In some embodiments, the present disclosure provides methods for immunotherapy comprising administering an effective amount of the engineered CD8 T cells and/or NK cells of the present disclosure. In one embodiment, a medical disease or disorder is treated by transfer of a CD8 T cell and/or NK cell population that elicits an immune response. In certain embodiments of the present disclosure, cancer or infection is treated by transfer of a CD8 T cell and/or NK cell population that elicits an immune response. Provided herein are methods for treating or delaying progression of cancer in an individual comprising administering to the individual an effective amount an adoptive cell therapy. The present methods may be applied for the treatment of solid cancers, hematologic cancers, and infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In some embodiments, T cells are autologous. However, the cells can be allogeneic. If the T cells are allogeneic, the T cells can be pooled from several donors. The cells are administered to the subject of interest in an amount sufficient to control, reduce, or eliminate symptoms and signs of the disease being treated.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a growth factor that promotes the growth and activation of the engineered T cells and/or NK cells is administered to the subject either concomitantly with the engineered T cells and/or NK cells or subsequently to the engineered T cells and/or NK cells. The growth factor can be any suitable growth factor that promotes the growth and activation of the engineered T-cells and/or NK cells. Examples of suitable growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

The engineered T cells or NK cells may be administered intravenously, intramuscularly, subcutaneously, intraperitoneally, by implantation, or by infusion. Intratumoral injection, or injection into the tumor vasculature is specifically contemplated for discrete, solid, accessible tumors. Local, regional or systemic administration also may be appropriate.

The appropriate dosage of the engineered immune cell therapy may be determined based on the type of disease to be treated, severity and course of the disease, the clinical condition of the individual, the individual's clinical history and response to the treatment, and the discretion of the attending physician. The therapeutically effective amount of immune cells for use in adoptive cell therapy is that amount that achieves a desired effect in a subject being treated.

The engineered immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

B. Pharmaceutical Compositions

Also provided herein are pharmaceutical compositions and formulations comprising immune cells (e.g., engineered T cells or NK cells) and a pharmaceutically acceptable carrier. Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX©, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

C. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. his approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the modified immune cells described herein may be included in the article of manufacture or kit. Alternatively, reagents for preparing modified immune cells as described herein may be included in the articles of manufacture or kit. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods for Examples 1-9

Mice. The Otub1-flox mice (in B6 genetic background) were generated using embryos obtained from The European Conditional Mouse Mutagenesis Program (EUCOMM, strain Otubt1^{tm1a(EUCOMM)Hmgu}). Otub1-flox mice were crossed with CD4^{Cre} transgenic mice (both in B6 genetic background and from Jackson Laboratories) to produce age-matched Otub1^{+/+}CD4^{Cre} (named WT) and Otub1^{fl/fl}CD4^{Cre} (named T cell-conditional Otub1 knockout or TKO) mice. The Otub1-flox mice were also crossed with ROSA26-CreER (Jackson Laboratories) to generate Otub1^{+/+}CreER and Otub1^{fl/fl}Cre-ER mice, which were then injected i.p. with tamoxifen (2 mg per mouse) in corn oil daily for four consecutive days to induce Cre function for generation of WT and induced Otub1 KO (iKO) mice. OT-I and Pmel1 TCR-transgenic mice, B6.SJL (CD45.1⁺), C57BL/6, Rag1-KO, and Il15ra-KO mice were from Jackson Laboratory. Experiments were performed with young adult (6-8 weeks) female and male mice except where indicated otherwise. All mice were in B6 genetic background and maintained in a specific pathogen-free facility of The University of Texas MD Anderson Cancer Center, and all animal experiments were done in accordance with protocols approved by the Institutional Animal Care and Use Committee of the University of Texas MD Anderson Cancer Center.

Cell lines. The HEK293T, B16F10, MC38 were from ATCC, and B16-OVA was provided by Qing Yi (Cleveland Clinic). The KIT225 T cell line stably transfected with IL-15Ra (15R-KIT) (Dubois et al., 2002) was provided by Dr. Sigrid Dubois (NCI/NIH) and cultured in RPMI 1640 medium supplemented with 10% FBS, antibiotics and human IL-2 (0.5 nM).

Plasmids, antibodies, and reagents. pMIGR1-HA-AKT was generated by inserting human AKT1 cDNA into the EcoRI and BglII sites of the retrovirus vector pMIGR1 downstream of an HA tag, and the AKT mutants (K8R, K14R, E17K) were created by site-directed mutagenesis. The pcDNA3 expression vectors for Flag-tagged Otub1 and Otub1 C91S mutant were provided by Dr. Danuek Durocher (Lunenfeld-Tanenbaum Research Institute), and Flag-Otub1 C91S/D88A mutant was generated by site-directed mutagenesis. pPRIChp-Otub1-HA and pPRIChp-Otub1C91S/D88A-HA were generated by inserting human Otub1 and Otub1 C91S/D88A into the pPRIChp-HA retroviral vector (provided by Dr. Patrick Martin, University of Nice Sophia Antipolis). PRK5-HA-ubiquitin WT, K63, and K48 were obtained from Addgene (Plasmid #17608, #17605, #17606). Ubiquitin K63 and K48 harbor lysine-to-arginine substitutions at all lysines, except lysine 63 and lysine 48, respectively. pLenti puro HA-ubiquitin was obtained from Addgene (plasmid #74218), and pLenti puro HA-Ub-AKT was generated by inserting human AKT1 cDNA into pLenti puro HA-ubiquitin immediately downstream of the ubiquitin cDNA. pLenti puro HA-Ub-AKT K14R was created by site-directed mutagenesis. pLenti puro HA-UbK63-AKT, and HA-UbK63-AKT K14R were generated by replacing WT ubiquitin with UbK63 in the pLenti HA-Ub-AKT and HA-Ub-AKT K14R vectors. T7-AKT was generated by inserting human AKT1 cDNA into the BamH1 and XbaI sites of T7-RelA vector (Addgene, #21984) to replace the RelA cDNA.

Functional grade anti-mouse (m) CD3_{ε} (145-2C11) and anti-mCD28 (37.51) antibodies were from eBioscience. Goat anti-hamster IgG (H+L) was from Southern biotech. Mouse IL-15 monoclonal antibody (AIO.3) used for in vivo IL-15 neutralization was from eBioscience. Antibodies for AKT1 (B-1; used for immunoblotting assays), ERK1/2 (K-23), Ubiquitin (P4D1), SLP76 (H-300), Zap70 (1E7.2), P85α (B-9) and PTEN (A2B1) were from Santa Cruz Biotechnology. Anti-AKT (40D4; used for IP) was from Cell Signaling, and anti-Otub1 (EPR13028(B)) was from Abcam. Anti-Actin (C-4), and horseradish peroxidase-conjugated anti-Flag (M2) were from Sigma-Aldrich. Antibodies for phospho-AKT1 S473 (D9E), phospho-AKT1 T308 (C31E5E), phospho-Fox01 Thr24/FoxO3a Thr32, phospho-S6K1 Thr421/Ser424, phospho-S6 Ser235/236 (D57.2.2E), phospho-Stat5 Tyr694 (C11C5), phosphor-SLP76 Ser376, phosphor-Zap70 Tyr329/Syk Tyr352, S6K1 (49D7), S6 (54D2), Fox01 (C29H4), Foxo3a (75D8), HK2 (C64G5), α-Tubulin, IGF1Rβ (111a9), and Stat5 were from Cell Signaling Technology. Horseradish peroxidase-conjugated anti-hemagglutinin (HA-7) was from Roche. The anti-CD8 (YTS169.4) and anti-NK1.1 (PK136) neutralizing antibodies were purchased from BioXCell.

Fluorescence-labeled antibodies for mCD4 (L3T4), mCD8 (53-6.7), mCD3 (145-2C11), CD44 (IM7), mCD62L (MEL-14), mTCRβ (H57-597), mCD45.1 (A20), mCD45.2 (104), mCXCR3 (CXCR3-173), mFoxp3 (FJK-16S), mCD45 (30-F11), mNK 1.1 (PK136), mCD11c (N418), mMHCII (M5/114.15.2), mCD64 (X54-5/7.1), mCD11b (M1/70), mIL-2 (JES6-SH4), mTNF (MP6-XT22) mGranzyme B (NGZB) and mIFN-γ (XMG1.2) were purchased from eBioscience. mCD24 (M1/69) and mCD103 (M290) were from BD and mCCL5(2E9/CCL5) was ordered from Biolegend. Glut1 (EPR3915) was from abcam.

Recombinant mouse IL-15, IL-2, IL-12, IL-18 and human IL-15 cytokines were from R&D. Human IL-2 were requested from NCI. The ELISA reagents for mouse IL-2, TNF, IFN-γ were from eBioscience. PIP3 beads and ELISA kits for detecting the activity of PI3K and PTEN were from Echelon. The GP10025-33 and OVA257-264 were ordered from ANAspec. The AKT inhibitor 1/2 (AKTi) was from Calbiochem.

Flow cytometry analysis and cell sorting. Single-cell suspensions of splenocytes and lymph node cells were subjected to flow cytometry analysis and cell sorting as previously described (Yu et al., 2015) using FACS fortessa and FACSAria (BD Biosciences). For intracellular cytokine staining (ICS) assays, T cells isolated from spleen, draining lymph nodes, or tumors of mice or from in vitro cultures were stimulated for 4 hours with PMA (50 ng/mL) and ionomycin (500 ng/mL) in the presence of monensin (10 μg/mL) during the last hour. The stimulated cells were fixed in 2% paraformaldehyde and permeablized in 0.5% saponin and then subjected to cytokine staining flow cytometry analyses. FACS data were analyzed in FlowJo 9.7.7 and proliferation index of CFSE labeled cells were calculated in FlowJo 10 proliferation modeling module. Gating strategies are summarized in FIG. 16.

L. monocytogenes infection. Age- and sex-matched WT and KO mice (6-8 wk old) were infected i.v. with 1×10⁵ colony-forming units of OVA-expressing recombinant L. monocytogenes (LM-OVA) (Pearce & Shen, 2007) (provided by Dr. Hao Shen, University of Pennsylvania). One day 7 post-infection, the mice were sacrificed for analysis of OVA-specific CD8 effector T cells in the spleen. Briefly, splenocytes were stimulated for 6 h with 10 μg/ml of OVA257-264 peptide (SIINFEKL, Genemed Synthesis), in the presence of a protein transport inhibitor, monensin, during the last hour, and then subjected to intracellular IFN-γ staining and flow cytometry analysis. 2×10⁴ colony-forming units of LM-OVA were used to infect WT OT-I and Otub1-TKO OT-I mice. On day 7 post-infection, splenocytes were collected and stimulated for 6 h with OVA257-264 peptide (10 μg/ml), with monensin being added during the last hour, and then subjected to intracellular IFN-γ staining and flow cytometry analysis.

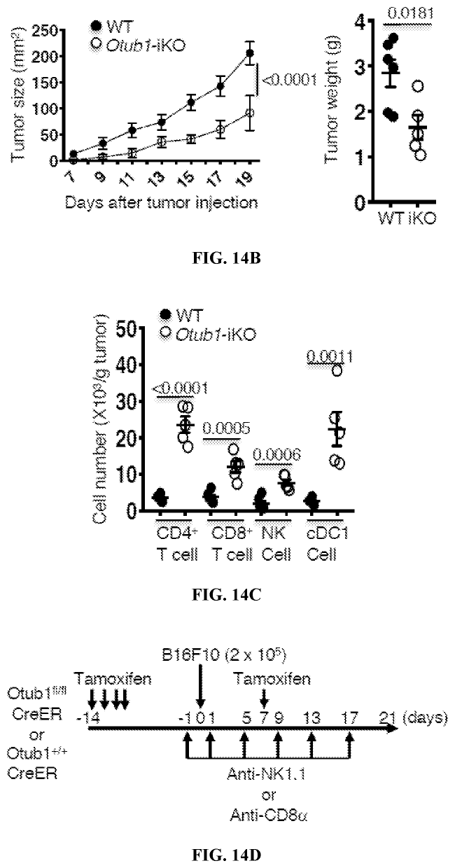

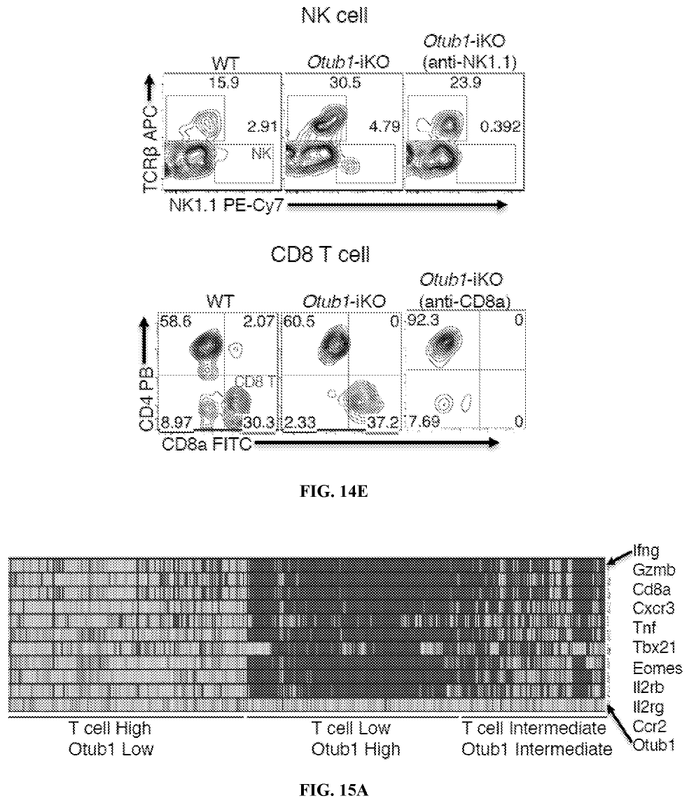

Tumor models. Age- and sex-matched WT and Otub1-TKO or WT and Otub1-iKO mice were injected s.c. with 2×10⁵ murine melanoma cells B16F10 or B16-OVA or with 2×10⁶ MC38 colon cancer cells and monitored for tumor growth. Mice were sacrificed and considered lethal when their tumor size reached 225 mm² based on protocols approved by the Institutional Animal Care and Use Committee of the University of Texas MD Anderson. At the indicated time point, all mice were sacrificed for flow cytometric analysis of immune cells from both the draining lymph nodes and tumors. For CD8 T cell and NK cell depletion experiments, age- and sex-matched WT and Otub1-iKO mice were inoculated s.c. with 2×10⁵ B16F10 melanoma cells and also injected i.p. with an anti-CD8 (clone YTS169.4) and anti-NK1.1 (clone PK136) neutralizing antibodies (100 μg) as depicted in FIG. 14D.

Adoptive cell therapy (ACT) was performed using Pmel1 CD8 T cells recognizing the B16 melanoma antigen gp100. Briefly, splenocytes were isolated from WT Pmel1 or Otub1-TKO Pmel1 mice and stimulated in vitro using plate-coated anti-CD3 (1 μg/ml) and soluble anti-CD28 (1 μg/ml) antibodies. The culture was provided with mIL-2 (10 ng/ml) on day 2, and CD8 T cells were purified from the culture on day 5 and used for adoptive transfer experiment. To generate tumor-bearing mice, WT B6 mice were injected s.c. with B16F10 melanoma cells. After four days, the tumor-bearing mice were subjected to whole-body irradiation (500 rads, ¹³⁷Cs irradiator) to induce lymphodepletion. One day after the irradiation, the mice were injected with the in vitro activated WT Pmel1 or Otub1-TKO Pmel1 CD8 T cells (6×10⁵). Control mice were not irradiated or injected with Pmel1 T cells. Tumor size was measured every other day for the indicated time period.

Mixed bone marrow and mixed T cell adoptive transfer. Bone marrow cells (2×10⁶) isolated from Otub1-TKO (CD45.2⁺) mice were mixed with bone marrow cells from WT B6.SJL (CD45.1⁺) mice in 1:1 ratio and adoptively transferred into irradiated (1000 rad) Rag1-KO mice. After 6 weeks, the bone marrow chimeric mice were sacrificed for analyzing the homeostasis of T cells derived from WT (B6.SJL) and Otub1-TKO bone marrows by flow cytometry based on the CD45.1 and CD45.2 congenital markers.

For mixed T cell transfer, WT (CD45.1⁺) and Otub1-TKO (CD45.2⁺) naive CD8 T cells (WT: CD45.1⁺; TKO: CD45.2⁺) or WT and Otub1-TKO naive OT-I CD8 T cells (WT OT-I: CD45.1⁺CD45.2⁺; TKO OT-I: CD45.2⁺) were labeled with CFSE dye, mixed in 1:1 ratio, and adoptively transferred into WT and Il15ra-KO mice. The transferred WT and Otub1-TKO CD8 T cells were analyzed by flow cytometry at the indicated time point. In some experiments, the Il15ra^{+/+} and Il15ra^{-/-} recipient mice were sublethally irradiated (600 rads, ¹³⁷Cs irradiator) to examine the role of IL-15 in mediating lymphopenic proliferation of CD8 T cells.

Metabolic assays. OCR and ECAR were measured with an XF96 extracellular flux analyzer (Seahorse Bioscience) following the manufacturer's instruction. Briefly, WT or Otub1-TKO CD8 naive T cells, either freshly isolated or in vitro activated with anti-CD3 plus anti-CD28 (for 24 h), were seeded in XF96 microplates (150,000 cells/well). The plates were quickly centrifuged to immobilize the cells. After incubation in a non-buffered assay medium (Seahorse Biosciences) in a non-CO₂ incubator for 30 min, the cells were subjected to glycolysis assays with a XF glycolysis stress test kit (Seahorse Biosciences). Initial measurement of ECAR was done when cells were incubated in a glycolysis stress test medium without glucose to record the baseline. Glucose (10 mM) was then injected to induce ECAR, reflecting glycolysis rate under basal conditions. Subsequently, oligomycin (1 μM) was injected to inhibit mitochondrial ATP production and shift the energy production to glycolysis, thereby measuring the maximum glycolytic capacity (also called stressed ECAR). Finally, a glucose analog, 2-deoxy-glucose (2-DG, 100 mM) was injected to inhibit glycolysis through targeting glucose hexokinase, resulting in decreased ECAR that served as a measure to confirm the glycolysis-dependence of the detected ECAR. Inhibitor studies were carried out by culturing the cells in 24-well plates (4×10⁶ cells/well) in the presence of indicated concentrations of AKT1/2 inhibitor or DMSO.

The Mito stress test kit (Seahorse Biosciences) was used to measure OCR under different conditions. After initial measurement of baseline OCR, 1 μM oligomycin was injected to calculate ATP-linked respiration, followed by injection of the protonophore FCCP (0.25 μM) that uncoupled oxygen consumption from ATP production to obtain maximal OCR (also called stressed OCR). Lastly, 0.5 M rotenone/antimycin A was injected to inhibit complex I and III and shut down ETC respiration for measuring non-mitochondrial respiration.

T-cell and NK cell purification and in vitro treatments. CD8 and CD4 T cells were isolated from splenocytes with anti-CD8- or anti-CD4-conjugated magnetic beads (Miltenyi), and naive CD8 or CD4 T cells were further purified by FACS sorting to get CD44^{lo}CD62L^{hi} population. The naive T cells were stimulated in replicate wells of 96-well plates (1×10⁵ cells per well) for 66 h, and the culture supernatants were analyzed by ELISA (eBioScience).

NK cells were isolated from splenocytes with NK cell isolation kit (Mietenyi). Purified NK cells were stimulated with IL2 (5 ng/ml), IL12 (10 ng/ml), and IL18 (10 ng/ml) for the indicated time periods and then subjected to flow cytometric analysis of intracellular granzyme B and CCL5.

RNA-sequencing analysis. Naïve CD8 T cells were isolated from the spleen of young (6-8 wk old) WT OT-I and Otub1-TKO OT-I mice and were either immediately lysed for RNA preparation or activated for 24 h with anti-CD3 (1 μg/ml) plus anti-CD28 (1 μg/ml). Total RNA was isolated with TRIzol (Invitrogen) and subjected to RNA-sequencing analysis using an Illumina sequencer in the Sequencing and Microarray Facility of the University of Texas MD Anderson Cancer Center. The raw reads were aligned to the mm10 reference genome (build mm10), using Tophat2 RNASeq alignment software. The mapping rate was 70% overall across all the samples in the dataset. HTseq-Count was used to quantify the gene expression counts from To-phat2 alignment files. Differential expression analysis was performed on the count data using R package DESeq2. P-values obtained from multiple binomial tests were adjusted using false discovery rate (Benjamini-Hochberg). Significant genes are defined by a Benjamini-Hochberg corrected p-value of cut-off of 0.05 and fold-change of at least two. RNA-sequencing data were analyzed by Genesis (available at genome.tugraz.at/) and multiplot (available at genepattern.broadinstitute.org/gp/pages/login.jsf). RNA sequencing data were deposited to Gene Expression Omnibus.

Real-time quantitative PCR. RNA was extracted with TRIzol reagent from isolated WT OT-I or Otub1-TKO OT-I CD8 T cells. The RNA samples were subjected to quantitative PCR analyses using the SYBR regent (Bio-Rad). The expression of individual genes was calculated by a standard curve method and was normalized to the expression of Actb. Gene-specific primer sets used in this study (all for mouse genes) are listed in Table 1.

TABLE 1
Real-time quantitative PCR primers

| | | | Product |
|---|---|---|---|
| | | | Size |
| Gene | Forward Primer Sequence | Reverse Primer Sequence | (bp) |
| Actin | CGTGAAAAGATGACCCAGATCA | CACAGCCTGGATGGCTACGT | 71 |
| | (SEQ ID NO: 9) | (SEQ ID NO: 10) | |
| Otub1 | GTAGCGACTCCGAAGGTGTT | ACCAGAGGATTCTGCACAGC | 100 |
| | (SEQ ID NO: 11) | (SEQ ID NO: 12) | |
| Cxcr3 | AGCACCAGCCAAGCCATGTA | CGTAGGGAGAGGTGCTGTTTT | 97 |
| | (SEQ ID NO: 13) | (SEQ ID NO: 14) | |
| Ccl5 | GCAGTCGTGTTTGTCACTCG | AGAGCAAGCAATGACAGGGA | 151 |
| | (SEQ ID NO: 15) | (SEQ ID NO: 16) | |
| Bcl2 | TCTGTGCACTGTGCATCTCTC | GACTTGGTGCATGGAACACTG | 121 |
| | (SEQ ID NO: 17) | (SEQ ID NO: 18) | |
| Eomes | TGAATGAACCTTCCAAGACTCAGA | GGTTATGGTCGATCTTTAGCTG | 108 |
| | (SEQ ID NO: 19) | (SEQ ID NO: 20) | |
| Runx3 | TGTCAGCGTGCGACATGGCT | GAGTGAAGCGGCGGCTGGTG | 99 |
| | (SEQ ID NO: 21) | (SEQ ID NO: 22) | |
| Runx2 | ATACCCCCTCGCTCTCTGTT | ACATAGGTCCCCATCTGCCT | 81 |
| | (SEQ ID NO: 23) | (SEQ ID NO: 24) | |
| Ccl2 | GGGATCATCTTGCTGGTGAA | AGGTCCCTGTCATGCTTCTG | 127 |
| | (SEQ ID NO: 25) | (SEQ ID NO: 26) | |
| Ccr5 | AGACATCCGTTCCCCCTACA | GCAGGGTGCTGACATACCAT | 107 |
| | (SEQ ID NO: 27) | (SEQ ID NO: 28) | |
| Cxcr4 | CCATGGAACCGATCAGTGTGA | TTTTCATCCCGGAAGCAGGG | 106 |
| | (SEQ ID NO: 29) | (SEQ ID NO: 30) | |
| Cd44 | CTCAGGAGCCCACAACGAGTGC | TCTGGGCTTCTTGCCTCTTGGGT | 78 |
| | (SEQ ID NO: 31) | (SEQ ID NO: 32) | |
| Il12rb2 | CGGGAAGAGCTCTGGAGAACC | GCATTCTCTAACAGTCTGTGCC | 72 |
| | (SEQ ID NO: 33) | (SEQ ID NO: 34) | |
| Tbx21 | GCCAGGGAACCGCTTATATG | GACGATCATCTGGGTCACATTGT | 136 |
| | (SEQ ID NO: 35) | (SEQ ID NO: 36) | |
| Lef1 | TCATCACCTACAGCGACGAG | GGGTAGAAGGTGGGGATTTC | 104 |
| | (SEQ ID NO: 37) | (SEQ ID NO: 38) | |
| Slamf1 | GCCTCTTATGCTTCAAACAACA | CAGCAGCATTGCCAAACAGT | 99 |
| | (SEQ ID NO: 39) | (SEQ ID NO: 40) | |
| Ly6a | GAAACCCCTCCCTCTTCAGGA | AGGGCTGCACAGATAAAACTTC | 131 |
| | (SEQ ID NO: 41) | (SEQ ID NO: 42) | |
| Hk2 | GATCGCCGGATTGGAACAGA | GGTCTAGCTGCTTAGCGTCC | 97 |
| | (SEQ ID NO: 43) | (SEQ ID NO: 44) | |
| Glut1 | GCTGTGCTTATGGGCTTCTC | CACATACATGGGCACAAAGC | 114 |
| | (SEQ ID NO: 45) | (SEQ ID NO: 46) | |

Retroviral and lentiviral infections. Retroviral particles were prepared using the indicated pMIGR1-GFP-based or pPRIChp-aHA-mCherry based expression vectors, as previously described (Yu et al., 2015). For production of lentiviral particles, ITEK293T cells were transfected (by calcium method) with pGIPZ lentiviral vectors encoding human Otub1-specific shRNAs (the binding site for shRNA #2 is: 5′-UCCGACUACCUUGUGGUCU-3′ (SEQ TD NO: 1); the binding site for shRNA #4 is: 5′-AAGGAGUUGCAGCGGUUCA-3′ (SEQ ID NO: 2)) or a non-silencing control shRNA along with the packaging vectors psPAX2 and pMID2. 15R-KIT T cells were infected with the recombinant retroviruses or lentiviruses. After 48 h, the transduced cells were enriched by flow cytometric cell sorting based on GFP expression. For primary T cell infection, naive OT-I CD8 T cells were stimulated in 12-well plates for 24 h with plate-bound anti-CD3 (1 g/ml) plus anti-CD28 (1 μg/ml) in the presence of 10 ng/ml IL-15 and 5 ng/ml IL-2 and then infected twice (at 48 h and 72 h) with retroviruses. 24 h after the second retroviral transduction, the infected T cells were starved in a low serum (0.5% FBS) medium overnight and then stimulated IL-15 (60 ng/ml) for signaling assays.

Immunoblot, co-immunoprecipitation, and ubiquitination assays. For immunoblot analysis of protein phosphorylation, naive CD4 and CD8 T cells or 15R-KIT T cell line cells were stimulated with IL-15 (60 ng/ml), IL-2 (60 ng/ml), or IL-7 (60 ng/ml) for the indicated time periods and lysed in a kinase cell lysis buffer supplemented with phosphatase inhibitors (Reiley et al., 2007). T cell stimulation with TCR and CD28 agonistic antibodies was performed using a crosslinking method (Reiley et al., 2007). Briefly, the cells were incubated on ice with anti-CD3 (2 μg/ml) and anti-CD28 (2 μg/ml), followed by crosslinking with goat anti-hamster Ig (25 μg/ml) for different time periods at 37° C. and then immediately lysed as described above for immunoblot assays.

Co-immunoprecipitations was performed essentially as described (Xiao et al., 2001). Primary OT-I CD8 T cells or 15R-KIT T cell line cells were stimulated with IL-15 (80 ng/ml) for the indicated time periods and lysed in a kinase cell lysis buffer (Reiley et al., 2007). Cell lysates were immediately subjected to immunoprecipitation using the indicated antibodies followed by immunoblot analysis of the precipitated proteins. For ubiquitination assays, stimulated T cells or transiently transfected HEK293 cells were lysed in RIPA buffer [50 mM Tris-HCl, pH 7.4, 150 mM NaCl, 1% (vol/vol) Nonidet P-40, 0.5% (vol/vol) sodium deoxycholate, and 1 mM EDTA] supplemented with 6 M urea and 4 mM N-ethylmaleimide. Lysates were diluted 1 time with RIPA buffer and then subjected to AKT immunoprecipitation, followed by detection of ubiquitinated AKT by immunoblot.

Membrane protein detection. Membrane and cytosol protein fractions were isolated from CD4 and CD8 T cells or NK cells with Mem-Per Plus Kit (Thermo Fisher) and subjected to immunoblot assays. To test the role of IL-15 in mediating Otub1 membrane localization, OT-I mice injected (i.p.) with a mouse IL-15 neutralizing antibody (AIO.3; 200 μg/mouse) daily for three times, and CD8 T cells were isolated on day 4 for preparing membrane and cytosol protein fractions. In some experiments, a T cell adoptive transfer approach was used. Briefly, OT-I CD8 T cells were labeled with CFSE and adoptively transferred into Il15ra^{+/+} or Il15ra^{-/-} recipient mice. After 7 days, the OT-I CD8 T cells were isolated from recipient mice for membrane and cytosol protein preparations.

Immune signature and survival analysis of human cancer. To correlate the expression level of Otub1 with the level of CD8 effector T cells in human cancer, 10 well-defined CD8 T cell-associated genes were collected to form the immune signature. SKCM tumor samples (n=458), including clinical and mRNA expression information, were downloaded from oncolnc.org/ and the compiled dataset was submitted to GenePattern (available at genepattern.broadinstitute.org/gp/pages/login.jsf) to do unsupervised hierarchical clustering analysis. Survival data from different clusters were used to do Kaplan-Meier estimation in GraphPad Prism software.

Statistical analysis. For the tumor clinical scores, differences between groups were evaluated by two-way ANOVA with Bonferroni's post-test. For survival, differences between groups were evaluated by Log-Rank test. Other statistical analyses were performed by two-tailed unpaired T test using the Prism software. P values less than 0.05 were considered significant, and the level of significance was indicated as *P<0.05, **P<0.01, ***P<0.001, ****P<0.0001.

In animal studies, 3-4 mice were required for each group based on our calculation to achieve a 2.3-fold change (effect size) in two-tailed T-test with 90% power and a significance level of 5%. All statistical tests justified as appropriate, and the data met the assumptions of the tests. The variance was similar between the groups that are being statistically compared.

Data availability. RNA sequencing datasets were deposited to Gene Expression Omnibus with the accession code GSE126777.

### Example 1—T Cell-Specific Otub1 Deficiency Causes Aberrant Activation of CD8 T Cells

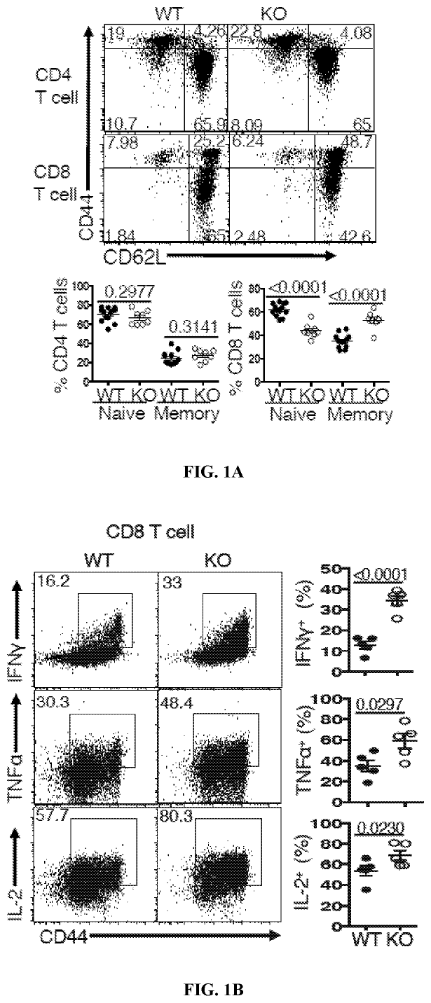

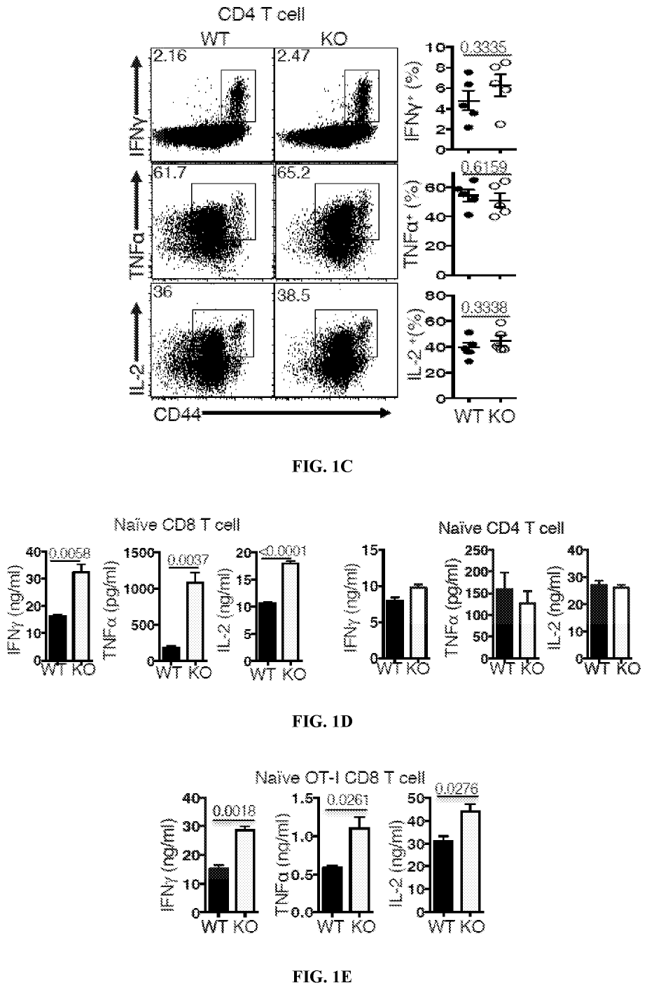

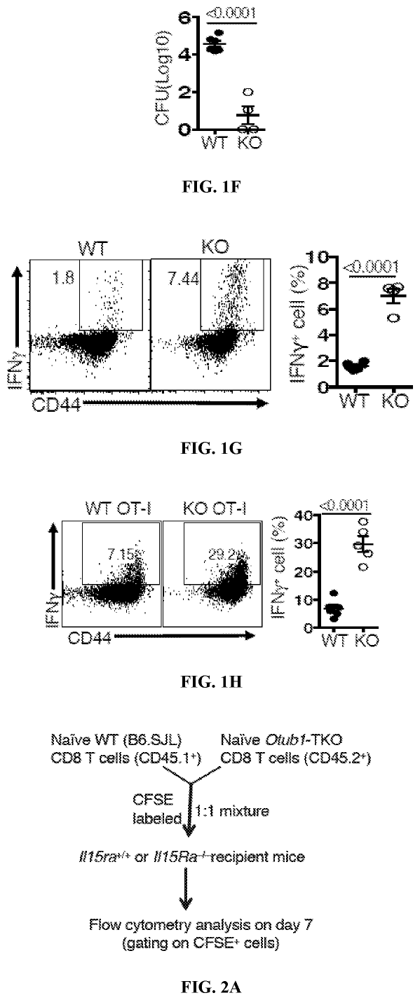

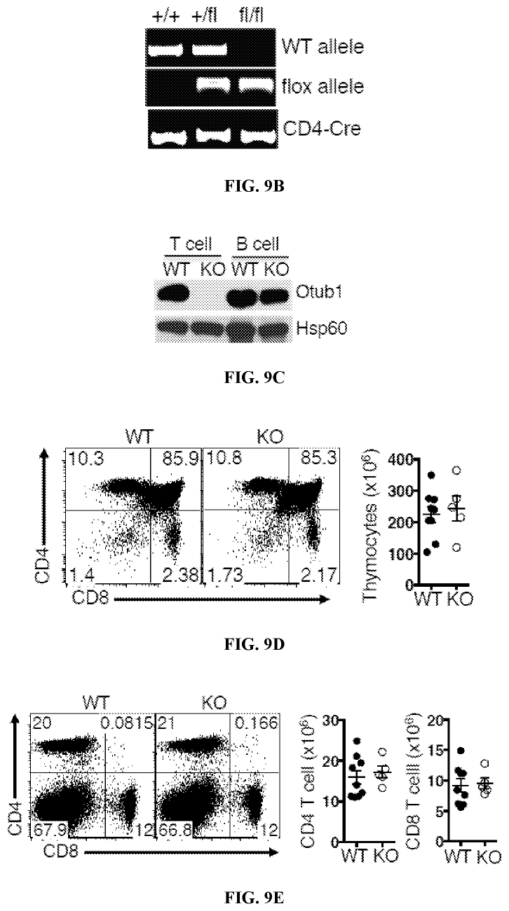

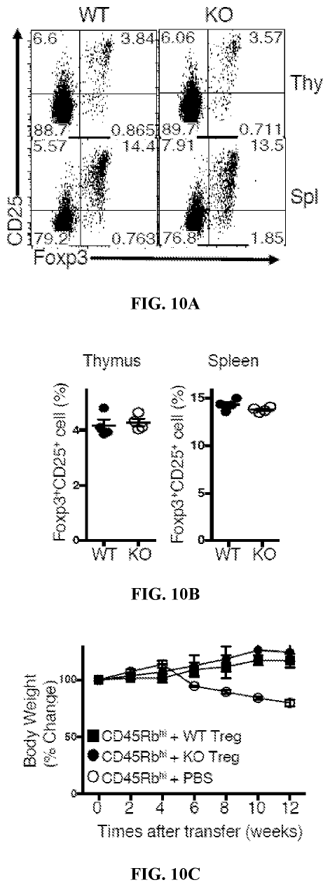

To study the function of Otub1 in T cells, Otub1 T cell conditional knockout (TKO) mice were generated (FIGS. 9A-C). The Otub1-TKO mice had normal frequencies of thymocyte and peripheral T cell populations (FIGS. 9D&E). However, they had increased frequencies of effector/memory-like (CD44hi) CD8 T cells producing effector cytokines, IFN-γ, TNF, and IL-2 (FIGS. 1A&B). Although Otub1 was similarly expressed in CD4 and CD8 T cells, Otub1 deficiency did not increase the frequency of CD4 effector/memory T cells (FIGS. 1A&C). The Otub1-TKO and wildtype (WT) mice had comparable frequencies of regulatory T cells (Treg cells), and the Otub1-deficient Treg cells were fully functional in suppressing naive CD4 T cells (FIGS. 10A-C). Mixed bone marrow adoptive transfer studies revealed that the Otub1-TKO CD8 T cells had increased frequencies of effector/memory-like population than WT CD8 T cells even in the same recipient mice (FIGS. 10D&E), suggesting a cell-intrinsic role for Otub1 in maintaining CD8 T cell homeostasis. Furthermore, the Otub1-deficient naive CD8 T cells were hyper-responsive to in vitro activation (FIG. 1D). Similar results were obtained with naive CD8 T cells from OT-I mice, producing CD8 T cells with a recombinant TCR specific for the chicken ovalbumin (OVA) peptide SIINFEKL21 (FIG. 1E). In contrast, Otub1 deficiency had no effect on naive CD4 T cell activation (FIG. 1D).

To examine the in vivo function of Otub1, a bacterial infection model was employed using a recombinant Listeria monocytogenes strain expressing chicken ovalbumin, LM-OVA. The Otub1-TKO mice displayed markedly enhanced immune responses against LM-OVA infection, as demonstrated by reduced liver bacterial load and increased frequencies of antigen-specific CD8 effector T cells producing IFN-γ (FIGS. 1F&G). Similar results were obtained using WT and Otub1-TKO OT-I mice producing OVA-specific CD8 T cells (FIG. 1H). These results suggest that Otub1 maintains CD8 T cell homeostasis and negatively regulates CD8 T cell activation.

### Example 2—Otub1 Regulates CD8 T Cell Responses to IL-15

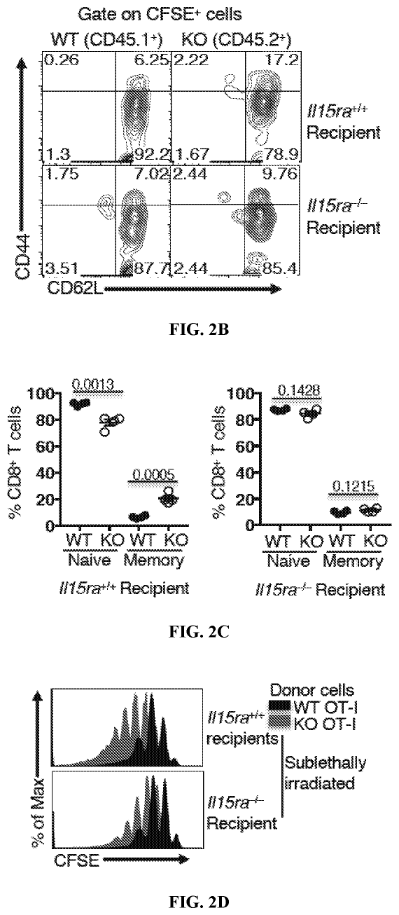

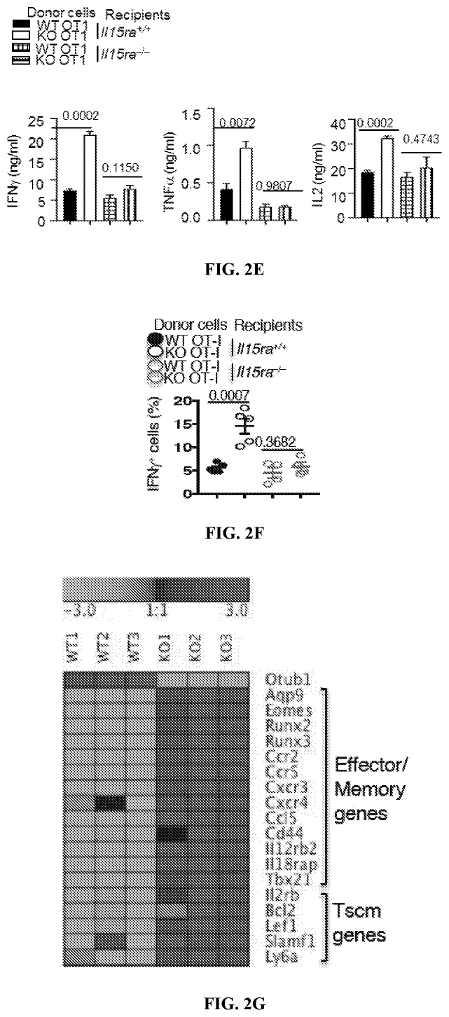

The γc family cytokines IL-7 and IL-15 are important for T cell homeostasis (Surh & Sprent, 2008; Lodolce et al., 2002). While IL-7 regulates both CD4 and CD8 T cells, IL-15 is particularly important for regulating CD8 T cells that express high levels of IL-15Rβ and γc (Schluns et al., 2000; Schluns & Lefrancois, 2003). Since Otub1 deficiency had selective effect on CD8 T cells (FIG. 1A), whether Otub1 played a role in regulating CD8 T cell responses to IL-15 by performing mixed CD8 T cell transfer using Il15ra^{+/+} or Il15ra^{-/-} recipient mice was tested (FIG. 2A). Since IL-15Ra is required for IL-15 transpresentation, T cells transferred to the Il15ra^{-/-} mice are defective in IL-15 stimulation (Burkett et al., 2003; Schlung et al., 2004). In the Il15ra^{+/+} recipients, Otub1-TKO CD8 T cells had much higher frequencies of memory-like T cells than WT CD8 T cells (FIGS. 2B&C). However, this phenotype was no longer significant in Il15ra^{-/-} recipients, suggesting a role for Otub1 in controlling CD8 T cell responses to IL-15 (FIGS. 2B&C).

The effect of Otub1 deficiency on IL-15-mediated CD8 T cell proliferation under lymphopenic conditions was also examined. OT-I CD8 T cells were used, since the OT-I TCR does not respond to commensal antigens and OT-I T cell expansion is mediated by homeostatic cytokines, predominantly IL-7 and IL-15 (Surh & Sprent, 2008; Goldrath et al., 2002). WT OT-I T cells proliferated to a similar level in Il15ra^{+/+} and Il15ra^{-/-} recipient mice (FIG. 2D), consistent with the involvement of both IL-7 and IL-15 in mediating lymphopenic T cell proliferation (Surh & Sprent, 2008; Schluns et al., 2000; Goldrath et al., 2002). However, the hyper-proliferation of the Otub1-TKO OT-I T cells was critically dependent on IL-15, since it was largely eliminated in the Il15ra^{-/-} recipient mice (FIG. 2D). These results further emphasize a crucial role for Otub1 in controlling CD8 T cell responses to the homeostatic cytokine IL-15.

### Example 3—IL-15 Primes CD8 T Cells for Activation Under the Control of Otub1

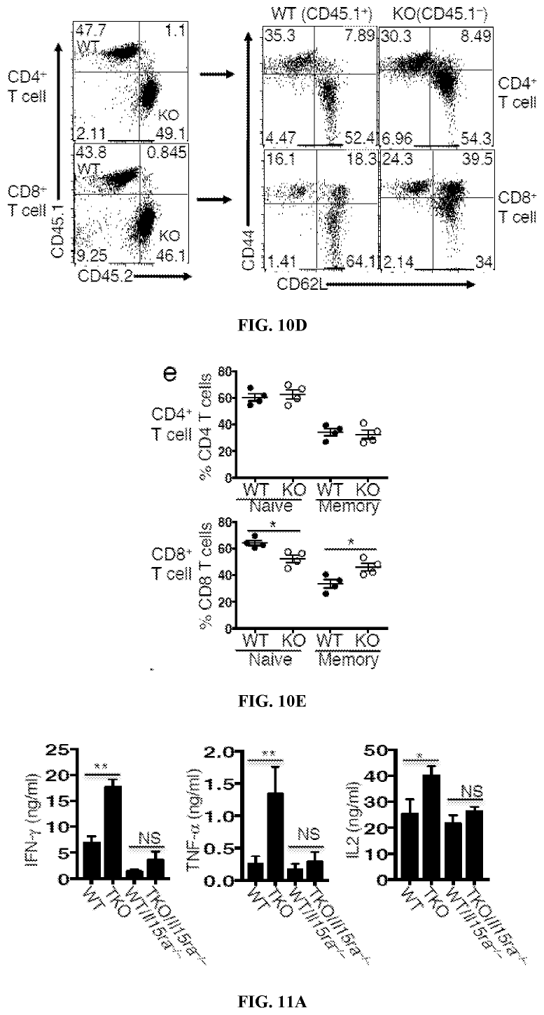

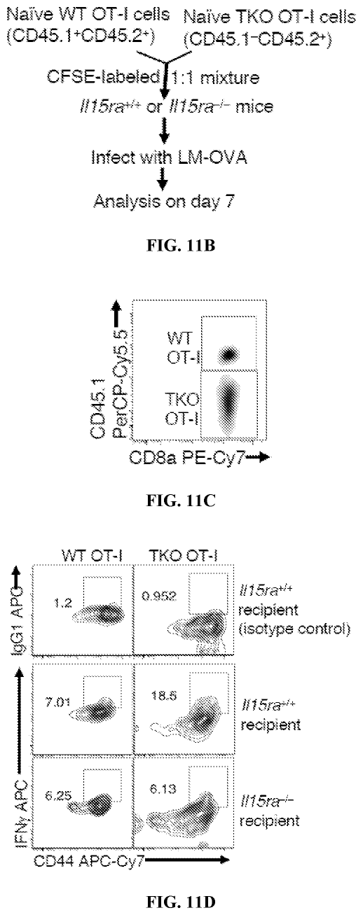

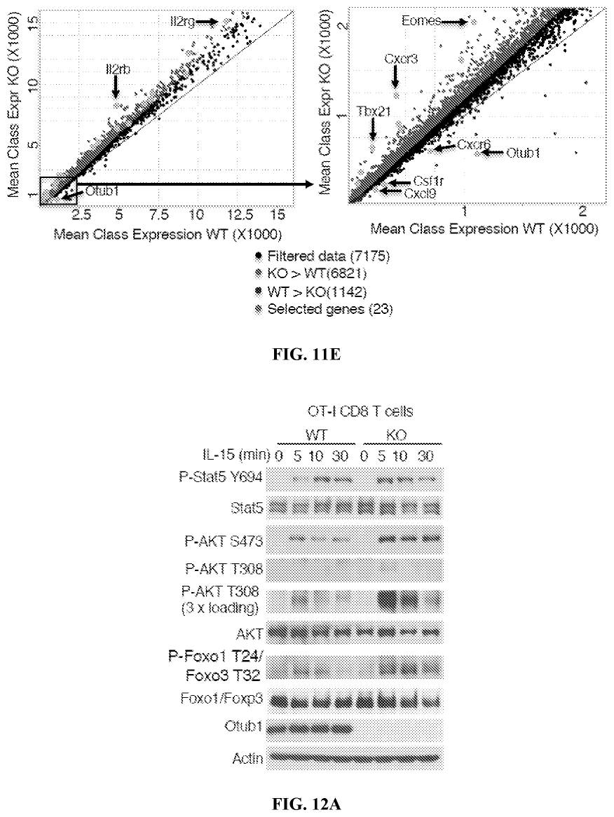

The fact that Otub1 deficiency promoted the activation of CD8 T cells by TCR-CD28 signals indicated that homeostatic exposure of CD8 T cells to IL-15 might prime them for activation by antigens. In further support of this, the hyper-responsive phenotype of Otub1-TKO CD8 T cells was detected in Il15ra^{+/+}, but not Il15ra^{-/-}, background (FIG. 11A). Furthermore, in a T cell adoptive transfer experiment, Otub1-TKO OT-I CD8 T cells isolated from Il15ra^{-/-} recipients, but not Il15ra^{-/-} recipients, displayed the hyper-activation phenotype (FIG. 2E). As an in vivo model, LM-OVA infection was performed using Il15ra^{+/+} or Il15ra^{-/-} mice adaptively transferred with a mixture of WT and Otub1-TKO naive OT-I CD8 T cells (FIGS. 11B&C). In Il15ra^{+/+} recipients, the Otub1-TKO OT-I T cells displayed a much stronger response to LM-OVA infection than the WT OT-I T cells, but this phenotype was not detected in the Il15ra^{-/-} recipients (FIGS. 2F& 11D). Thus, Otub1 controls IL-15-mediated priming of CD8 T cells for antigen-specific responses both in vitro and in vivo.

RNA sequencing revealed that the Otub1-TKO naive OT-I T cells had upregulated expression of a large number of genes under homeostatic conditions (FIG. 11E), including signatures associated with effector/memory functions and stem memory T cells (Tscm) (FIG. 2G). To examine whether this gene expression signature was dependent on IL-15 signaling, qRT-PCR analysis was performed using WT and Otub1-TKO CD8 T cells isolated from adoptively transferred Il15ra^{+/+} or Il15ra^{-/-} recipient mice (FIG. 2H). Within the Il15ra^{+/+} recipient mice, the Otub1-TKO CD8 T cells displayed upregulated expression of almost all of the genes analyzed compared to the WT CD8 T cells (FIG. 2H). However, within the Il15ra^{-/-} recipient mice, the WT and Otub1-TKO CD8 T cells no long displayed differences in gene expression, and both displayed reduced level of gene expression compared to CD8 T cells derived from the Il15ra^{+/+} recipient mice (FIG. 2H). Together, these results suggest that under homeostatic conditions, IL-15 primes CD8 T cells for responding to TCR-CD28 signals, which is negatively regulated by Otub1.

### Example 4—Otub1 Regulates NK Cell Maturation and Activation

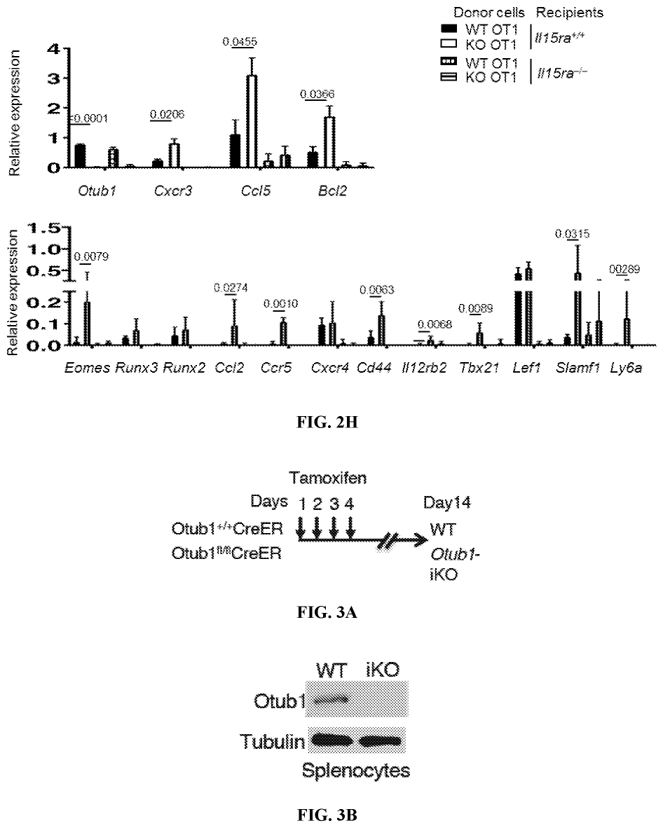

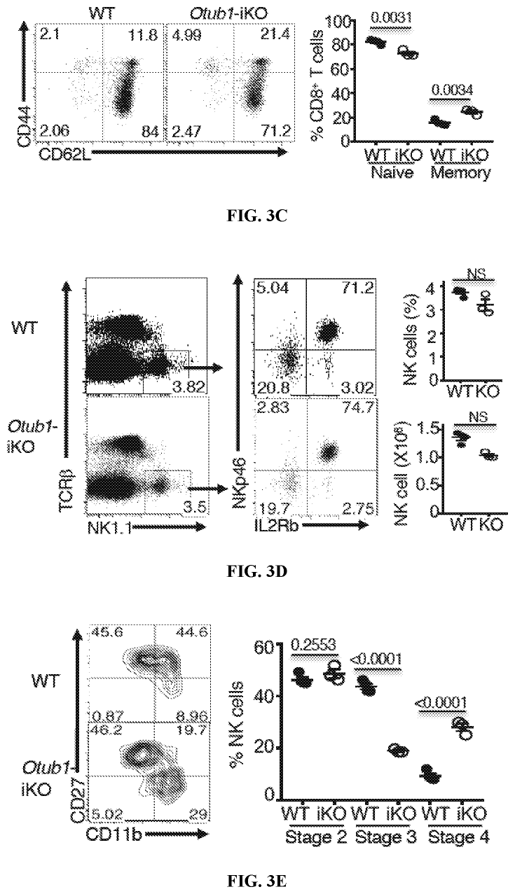

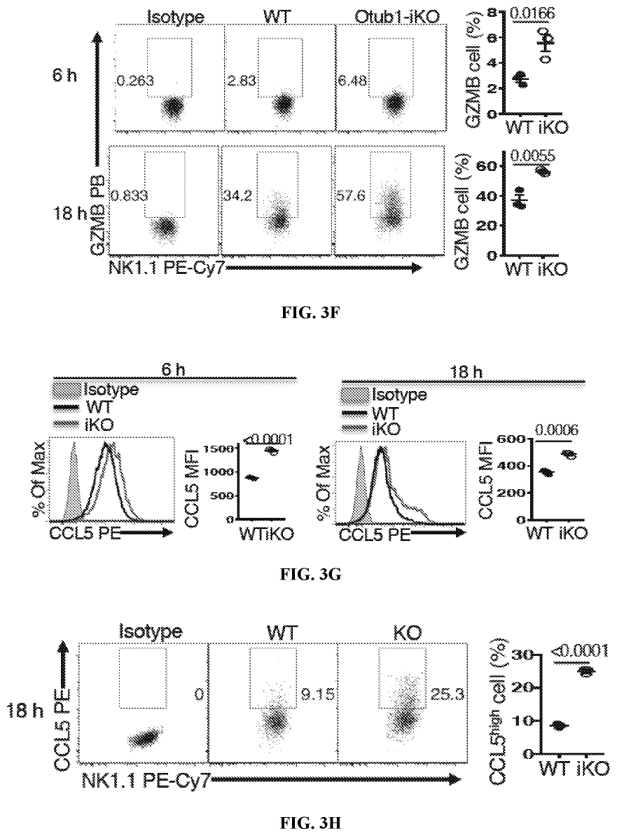

NK cells also express high levels of IL-15R β/γ heterodimer and rely on IL-15 for maturation and activation (Guillerey et al., 2016). Based on surface expression of CD11b and CD27, NK cells can be divided into four maturation stages: stage 1 (CD11b^{lo}CD27^{lo}), stage 2 (CD11b^{lo}CD27^{hi}), stage 3 (CD11b^{hi}CD27^{hi}), and stage 4 (CD11b^{hi}CD27^{lo}), with progressive acquisition of effector functions (Chiossone et al., 2009). IL-15 deficiency impairs generation of stage 3 and stage 4 NK cells, whereas IL-15 overexpression causes predominant accumulation of stage 4 NK cells (Polansky et al., 2016). To study the function of Otub1 in NK cell regulation, Otub1 was inducibly deleted in adult mice using a tamoxifen-inducible Cre (CreER) system (FIGS. 3A&B). As expected from the Otub1-TKO result (FIG. 1A), Otub1 induced KO (Otub1-iKO) mice had increased frequencies of memory-like CD8 T cells (FIG. 3C). Importantly, although the Otub1 deletion had no effect on total NK cell number in the spleen, it markedly increased the frequency of stage 4 mature NK cells (CD11b^{hi}CD27^{lo}) and concomitantly reduced stage 3 NK cells (CD11b^{hi}CD27^{hi}) (FIGS. 3D&E). Consistently, Otub1-iKO NK cells were hyper-responsive to cytokine-stimulated activation, detected based on production of Granzyme B and the chemokine CCL5 (FIGS. 3F-H), mediating NK cell effector function and recruitment of type 1 conventional dendritic cells (cDC1), respectively (Bottcher et al., 2018). These results suggest that Otub1 controls the maturation and activation of NK cells, further emphasizing the role of this DUB in regulating IL-15 responses.

### Example 5—Otub1 Regulates the AKT Axis of IL-15 Receptor Signaling

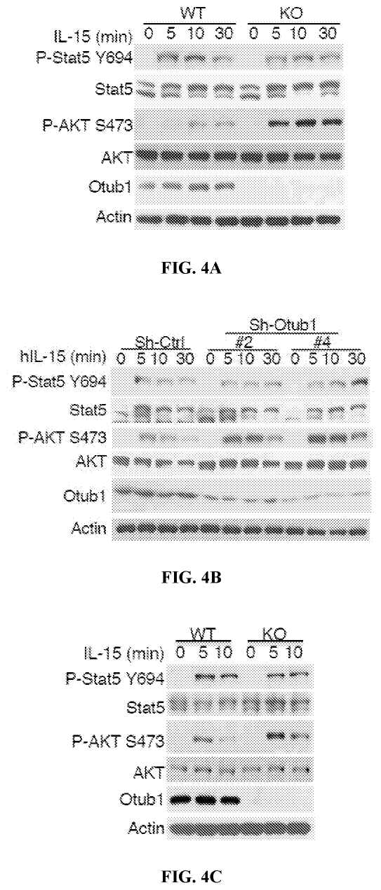

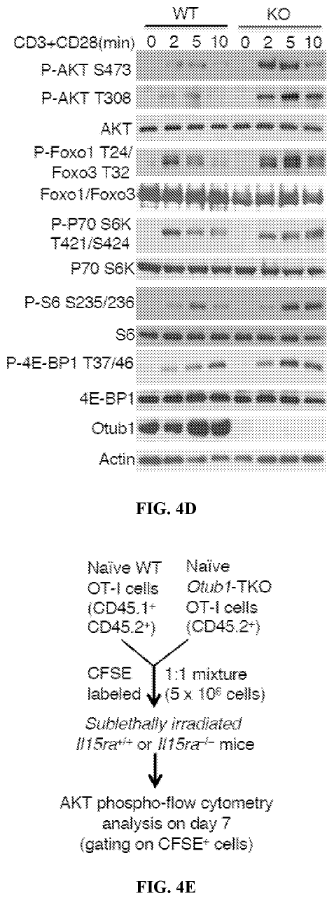

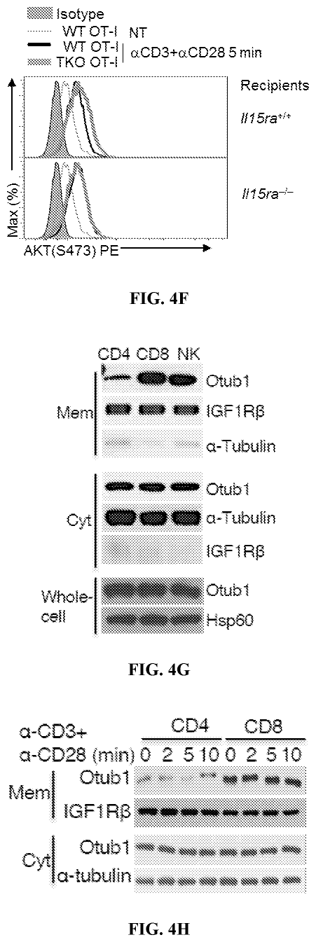

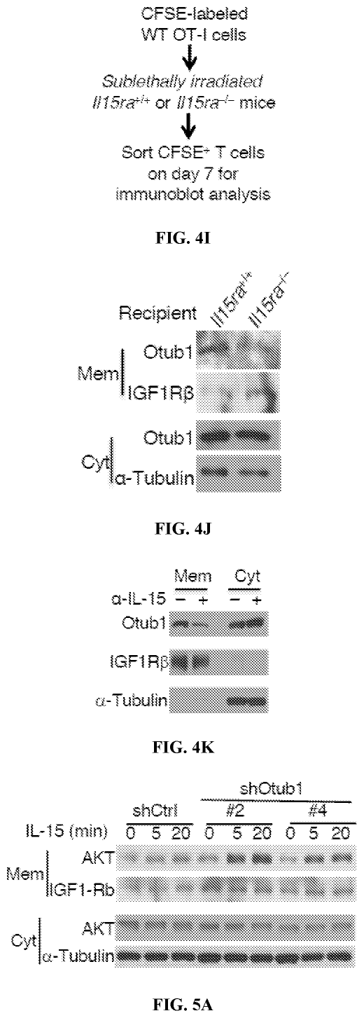

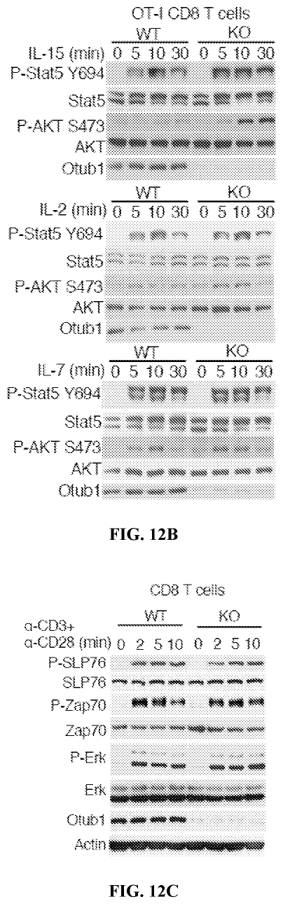

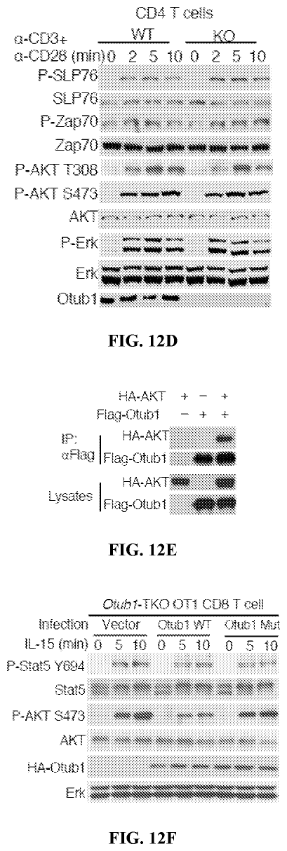

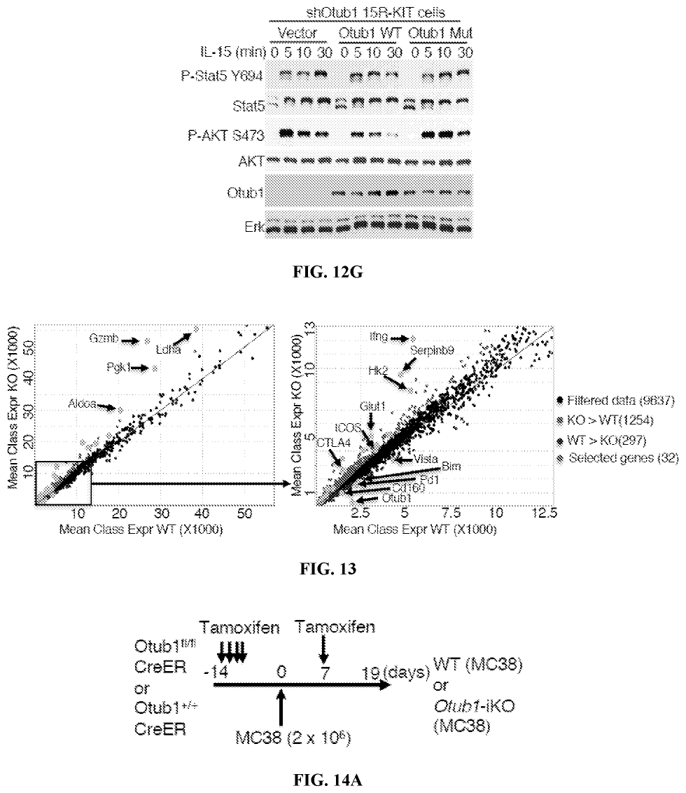

Stimulation of naive CD8 T cells with IL-15 triggered activation of the transcription factor STAT5 and the kinase AKT, as shown by their site-specific phosphorylation (FIG. 4A). Otub1 deficiency did not affect STAT5 activation but strikingly enhanced activation of AKT (FIG. 4A). AKT activation is mediated via its phosphorylation at threonine 308 (T308) and serine 473 (S473). AKT T308 phosphorylation is crucial for activation of the metabolic kinase mTORC1, whereas AKT S473 phosphorylation is required for phosphorylating and inactivating FOXO family of transcription factors, a mechanism that promotes CD8 T cell effector functions (Vadlakonda et al., 2013; Kim et al., 2012). The Otub1 deficiency enhanced IL-15-stimulated phosphorylation of AKT S473 as well as FOXO1 and FOXO3 (FIGS. 4A&12A). IL-15-stimulated AKT T308 phosphorylation was relatively weak, which required loading more cell lysates for clear detection (FIG. 12A). Nevertheless, the AKT T308 phosphorylation was also enhanced in Otub1-deficient CD8 T cells (FIG. 12A). Otub1 deficiency only had a weak effect on IL-2- and IL-7-stimulated AKT phosphorylation (FIG. 12B). Notably, the receptors of IL-2 and IL-15 share two common subunits, IL-2/IL-15Rβ and γc, although these two cytokines display different biological functions (Waldmann, 2015). These findings suggested signaling differences between these two closely related cytokines. The role of Otub1 in regulating IL-15-stimulated AKT activation was further demonstrated using an IL-15-responsive T cell line, 15R-KIT (human KIT-225 cell line stably transfected with IL-15Ra). Otub1 knockdown in 15R-KIT T cells strongly promoted IL-15-stimulated AKT phosphorylation (FIG. 4B). Furthermore, Otub1 deficiency in NK cells also profoundly enhanced IL-15-stimulated activation of AKT, but not activation of STAT5 (FIG. 4C). Thus, Otub1 controls the AKT axis of IL-15R signaling in both CD8 T cells and NK cells.

Since the Otub1-deficient CD8 T cells were hyper-responsive to TCR-CD28 stimulation in vitro and antigen-specific responses in vivo (FIG. 1D-H), the effect of Otub1 deletion on TCR signaling was examined. Otub1 deficiency did not influence the phosphorylation of the protein tyrosine kinase Zap70, the adaptor protein SLP76, and the MAP kinase ERK (FIG. 12C). However, Otub1 deficiency markedly enhanced TCR-CD28-stimulated activation of AKT and phosphorylation of several AKT downstream proteins, including the transcription factors Foxo1 and Foxo3 and the mTORC1 targets S6 kinase (S6K), ribosomal S6 protein, and 4E-BP1 (FIG. 4D). On the other hand, the Otub1 deficiency did not affect TCR-CD28-stimulated AKT signaling in CD4 T cells (FIG. 12D), consistent with the finding that Otub1 controlled the activation of CD8, but not CD4, T cells (FIG. 1D).

To examine whether the TCR-CD28-stimulated AKT hyper-activation in Otub1-deficient CD8 T cells was due to IL-15 priming, WT or Otub1-TKO naive OT-I T cells were adoptively transferred to Il15ra^{+/+} or Il15ra^{-/-} recipient mice and the transferred T cells were sorted for an AKT activation assay (FIG. 4E). The Otub1-TKO OT-I CD8 T cells isolated from Il15ra^{+/+}, but not Il15ra^{-/-}, recipient mice displayed hyper-activation of AKT (FIG. 4F), suggesting that IL-15 primes CD8 T cells for the AKT axis of TCR-CD28 signaling under the control of Otub1. In an effort to further explore the mechanism by which Otub1 selectively regulates AKT signaling in CD8 T cells, it was found that CD8, but not CD4, T cells contained abundant membrane-associated Otub1 (FIG. 4G). Like CD8 T cells, NK cells also contained a high level of membrane-associated Otub1 (FIG. 4G). The membrane association of Otub1 was not affected by TCR-CD28 signaling (FIG. 4H), but was critically dependent on IL-15 since it was diminished in CD8 T cells derived from Il-15Rα-deficient host in a T cell transfer study (FIGS. 4I&J). Antibody-mediated IL-15 neutralization in WT OT-I mice also inhibited Otub1 membrane localization in CD8 T cells (FIG. 4K). Since AKT activation occurs in various membrane compartments (Jethwa et al., 2015), these findings provide insight into the mechanism underlying the AKT-regulatory function of Otub1.

### Example 6—Otub1 Inhibits K63 Ubiquitination and the PIP3-Binding Function of AKT

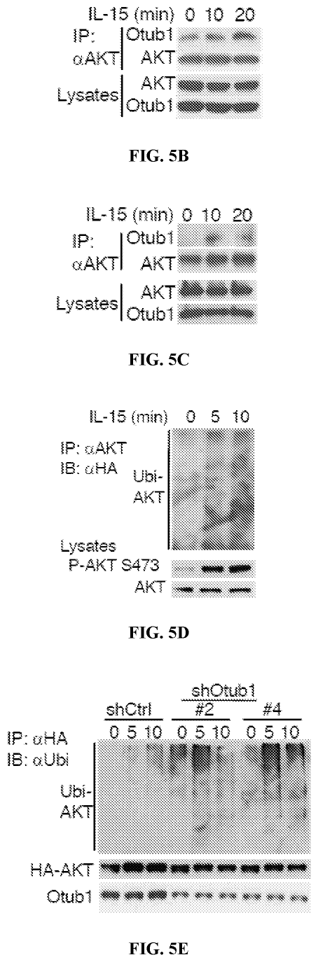

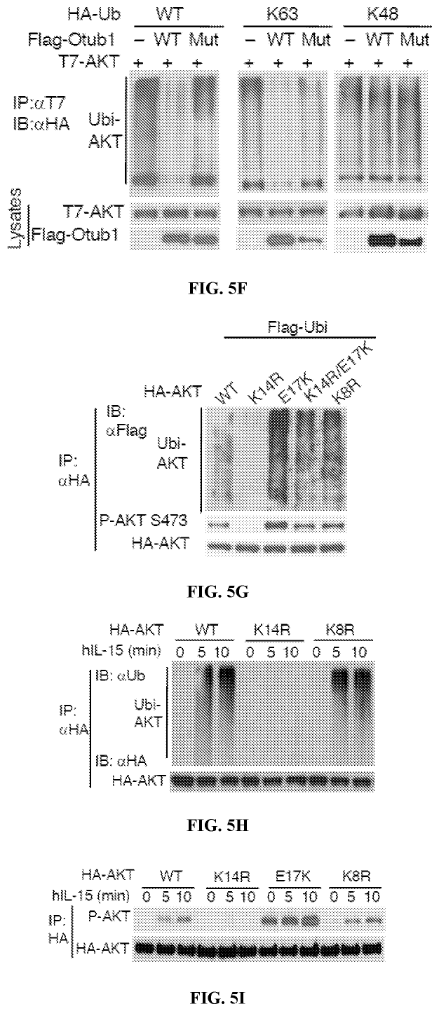

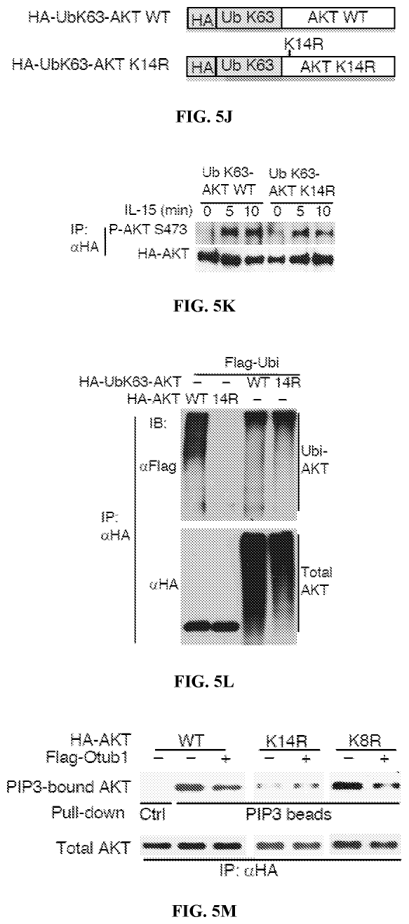

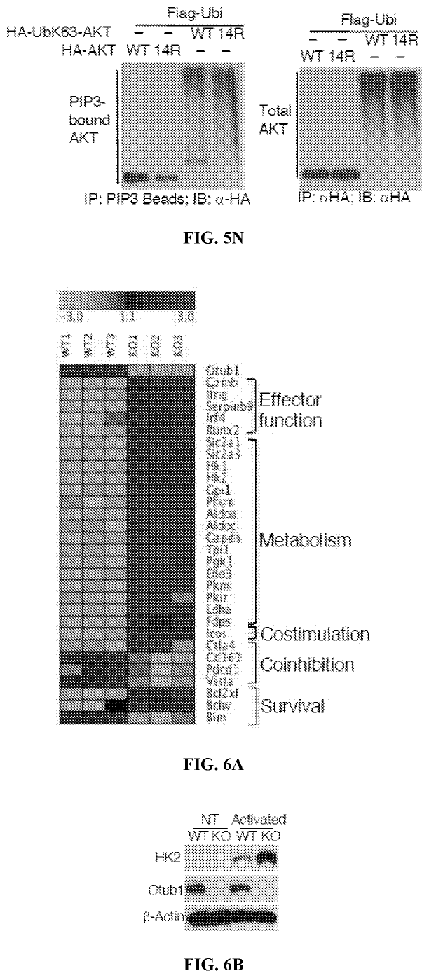

A key step in AKT activation is its recruitment to membrane compartments via interaction of its pleckstrin homology (PH) domain with the membrane lipid PIP3 (Cantley, 2002). Once in the membrane, AKT is phosphorylated at T308 and S473 by PDK1 and mTORC2, respectively. IL-15-stimulated membrane translocation of AKT was greatly enhanced by Otub1 knockdown (FIG. 5A). Otub1 knockdown had no obvious effect on the activity of AKT upstream regulators, PI3 kinase (PI3K) and PTEN, which catalyze the forward and reverse PIP3 generation reactions, respectively (Carnero et al., 2008). Interestingly, AKT was physically associated with Otub1 in 15R-KIT cells, which was strongly enhanced upon IL-15 stimulation (FIG. 5B). In primary OT-I CD8 T cells, the AKT-Otub1 interaction was barely detectable at steady state but was strongly induced by IL-15 (FIG. 5C). The Otub1-AKT binding was also readily detected under transfection conditions (FIG. 12E).

Since Otub1 is a DUB, it was next examined whether Otub1 regulated the ubiquitination of AKT. IL-15 stimulated ubiquitination of AKT, which was enhanced upon Otub1 knockdown (FIGS. 5D&E). Conversely, Otub1 overexpression inhibited AKT ubiquitination, which was efficient for K63-linked, but not K48-linked, polyubiquitin chains (FIG. 5F). A previous study identified three catalytic residues of Otub1: cysteine 91 (C91), aspartate 88 (D88), and histidine 265 (H265) (Balakirev et al., 2003). Mutation of C91 only moderately inhibited the function of Otub1, but simultaneous mutations of D88 and C91 generated an Otub1 mutant, D88A/C91S, that was unable to inhibit AKT ubiquitination (FIG. 5F). WT Otub1, but not D88A/C91S, was also able to suppress AKT activation in reconstituted Otub1-deficient CD8 T cells and Otub1-knockdown 15R-KIT cells (FIGS. 12F&G), thus suggesting that Otub1-mediated inhibition of AKT K63 ubiquitination contributes to the negative regulation of AKT activation.

TRAF6 is known to mediate growth factor-induced AKT ubiquitination at K8 and K14 in cancer cells (Yang et al., 2009). Mutation of K14 also abolished AKT ubiquitination under basal and IL-15-stimulated conditions (FIGS. 5G&H). However, mutation of K8 had no effect on AKT ubiquitination (FIGS. 5G&H). Consistently, mutation of K14, but not K8, abolished AKT phosphorylation (FIG. 5I), suggesting AKT K14 ubiquitination mediates its activation by IL-15. To further assess the function of AKT K63 ubiquitination, a K63 ubiquitin mutant (UbK63) was fused with AKT or AKT K14R at the N-terminus close to residue K14 (FIG. 5J). The UbK63-AKT fusion protein behaved like AKT in responding to IL-15 for phosphorylation (FIG. 5K). Fusion of UbK63 to AKT K14R largely rescued its defect in IL-15-stimulated phosphorylation as well as in ubiquitination (FIGS. 5K&L), suggesting that the fused UbK63 could serve as an acceptor ubiquitin for polyubiquitin chain formation and, thus, AKT activation.

AKT normally exists in a closed conformation due to the intramolecular interaction between its N-terminal PH domain and C-terminal kinase domain (Calleja et al., 2009). Since ubiquitination often causes conformation changes, it was hypothesized that AKT ubiquitination might promote its PIP3-binding activity. While WT AKT and AKT K8R displayed strong PIP3-binding activity, the AKT K14R mutant was defective in PIP3 binding (FIG. 5M). Moreover, Otub1 strongly inhibited the PIP3-binding activity of AKT WT and AKT K8R, but it did not affect the residual PIP3-binding activity of K14R (FIG. 5M). Fusion of UbK63 to AKT K14R, which restored its ubiquitination (FIG. 5L), completely restored its PIP3-binding function (FIG. 5N). These results suggest that Otub1 deubiquitinates AKT to interfere with the PIP3-binding and membrane translocation of AKT, thereby inhibiting its phosphorylation and activation.

### Example 7—Otub1 Regulates Important Gene Signatures and Metabolic Programing in Activated CD8 T Cells

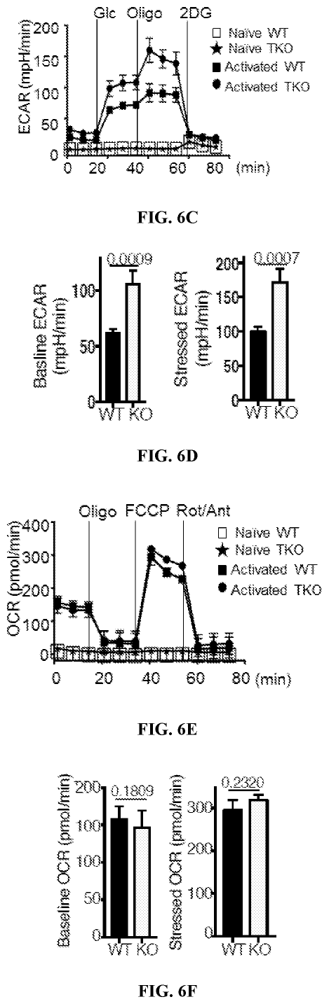

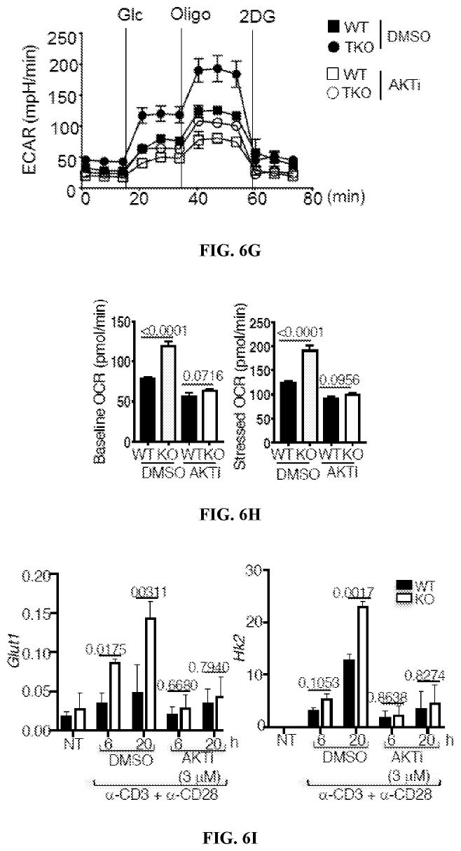

RNA sequencing analysis of in vitro activated CD8 T cells revealed that Otub1-deficient CD8 T cells had 1254 significantly upregulated and 297 significantly downregulated genes compared to WT CD8 T cells (FIG. 13). The upregulated genes included those involved in activation and effector function or survival of CD8 T cells (FIG. 6A). The major down-regulated genes included those encoding a pro-apoptotic factor, Bim, and immune checkpoint molecules (Pd1, Vista, and CD160) (FIG. 6A). The most striking result was the upregulated expression of a metabolic gene signature in the Otub1-deficient CD8 T cells, particularly those involved in the glycolytic pathway, such as glucose transporter 1 (Glut1, also called Slc2a1) and hexokinase 2 (Hk2) (FIGS. 6A&13). Immunoblot analyses confirmed the drastic upregulation of HK2, an enzyme catalyzing the first step of glycolytic pathway (Roberts & Miyamoto, 2015), in Otub1-TKO CD8 T cells (FIG. 6B). These findings are intriguing, since metabolic reprograming is a hallmark of T cell activation and required for the function of effector T cells (Pearce et al., 2013; Zheng et al., 2009; McKinney & Smith, 2018).

Next, Seahorse Extracellular Flux analyses was performed to measure extracellular acidification rate (ECAR) and oxygen consumption rate (OCR), indicators of aerobic glycolysis and oxidative phosphorylation, respectively (Pearce et al., 2013). Compared to WT CD8 T cells, Otub1-deficient CD8 T cells had enhanced ECAR and maximum glycolytic capacity (stressed ECAR) under activated conditions (FIGS. 6C&D). Unlike glycolysis, OCR was not significantly altered by the Otub1 deficiency (FIGS. 6E&F). Otub1 appeared to regulate glycolysis through controlling AKT, since a selective AKT inhibitor (AKTi) erased the ECAR differences between WT and Otub1-TKO CD8 T cells (FIGS. 6G&H). The AKT inhibitor also blocked TCR-CD28-stimulated hyper-expression of the glycolysis-regulatory genes, Glut1 and Hk2, and cytokine production in Otub1-TKO CD8 T cells (FIGS. 6I&J). These results suggest that Otub1 controls glycolysis induction in activated CD8 T cells via a mechanism that involves regulation of AKT signaling.

### Example 8—Otub1 Deficiency Impairs CD8 T Cell Self-Tolerance

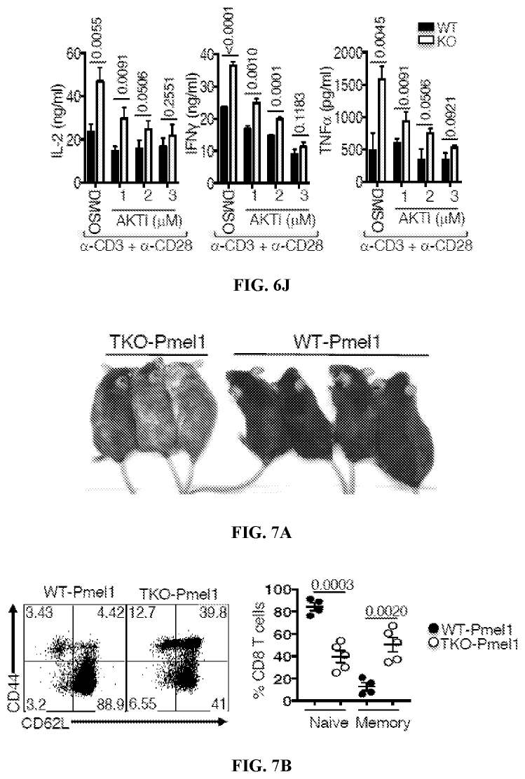

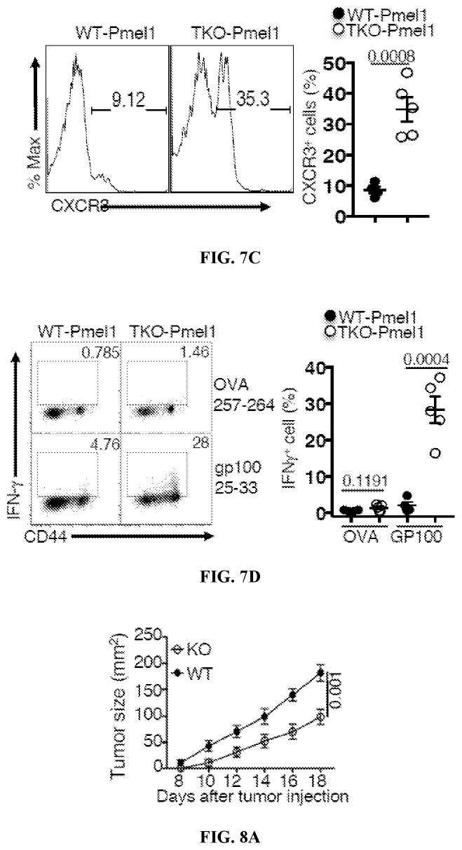

IL-15 is known to reduce the threshold of T cell activation and sensitizes CD8 T cells for responses to self-antigens (Deshpande et al., 2013; Huang et al., 2015). The role of Otub1 in regulating CD8 T cell self-tolerance was examined using a well-defined mouse model, Pmel1, producing CD8 T cells with a transgenic TCR specific for the melanocyte self-antigen, gp100 (Overwijk et al., 2003). The Pmel1 CD8 T cells are normally tolerant to the self-antigen gp100, and impaired self-tolerance causes a skin autoimmunity, vitiligo, characterized by hair depigmentation (Overwijk et al., 2003; Zhang et al., 2007). Although WT Pmel1 mice only developed minor vitiligo up to 9 months of age, 100% of the Otub1-TKO Pmel1 mice developed severe vitiligo, starting from around 3 months of age and becoming more severe over time (FIG. 7A). While the WT Pmel1 CD8 T cells were predominantly in a naive state, a large proportion of the Otub1-TKO Pmel1 CD8 T cells were activated, displaying CD44 and CXCR3 activation markers (FIGS. 7B&C). Furthermore, the Otub1-TKO, but not WT, Pmel1 T cells responded to in vitro restimulation with the antigen gp100, for IFN-γ production (FIG. 7D). These results suggest that Otub1 controls CD8 T cell responses to microbial antigens and self-antigens in vivo.

### Example 9—Otub1 Regulates Anticancer Immunity Via Both T Cells and NK Cells

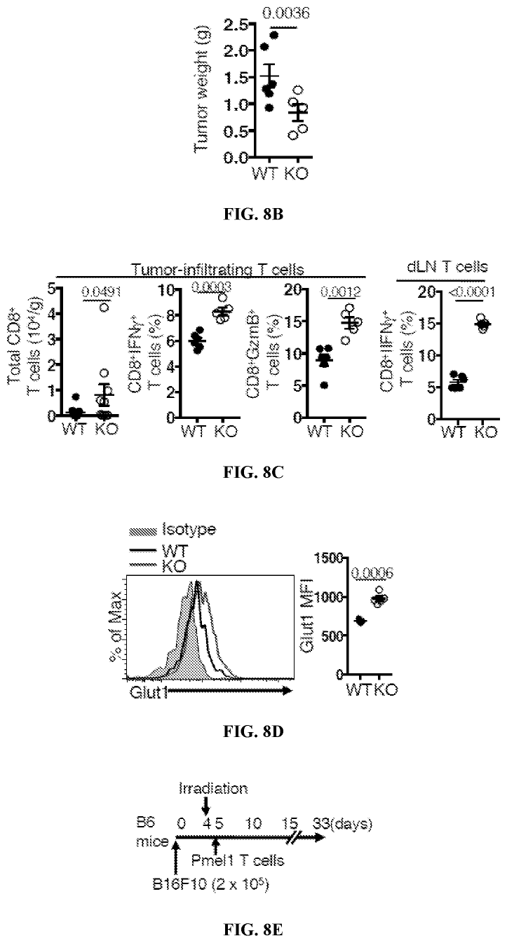

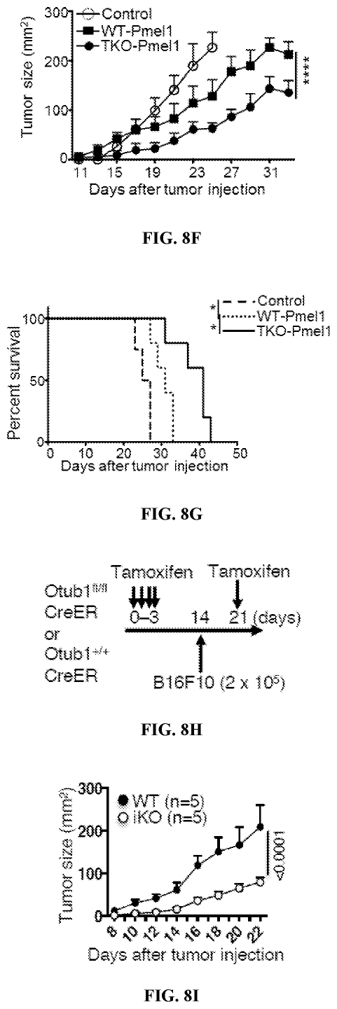

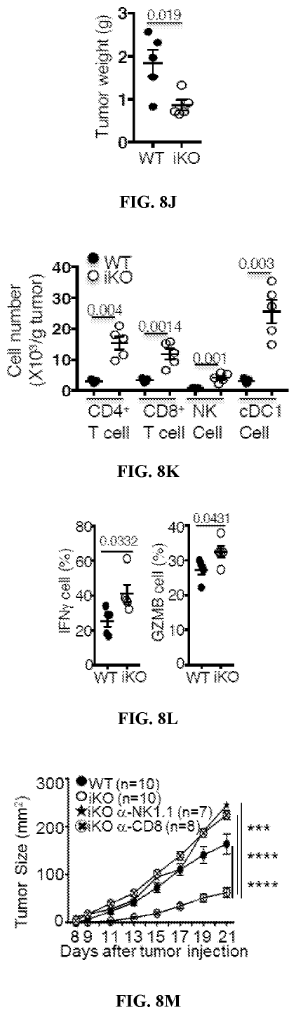

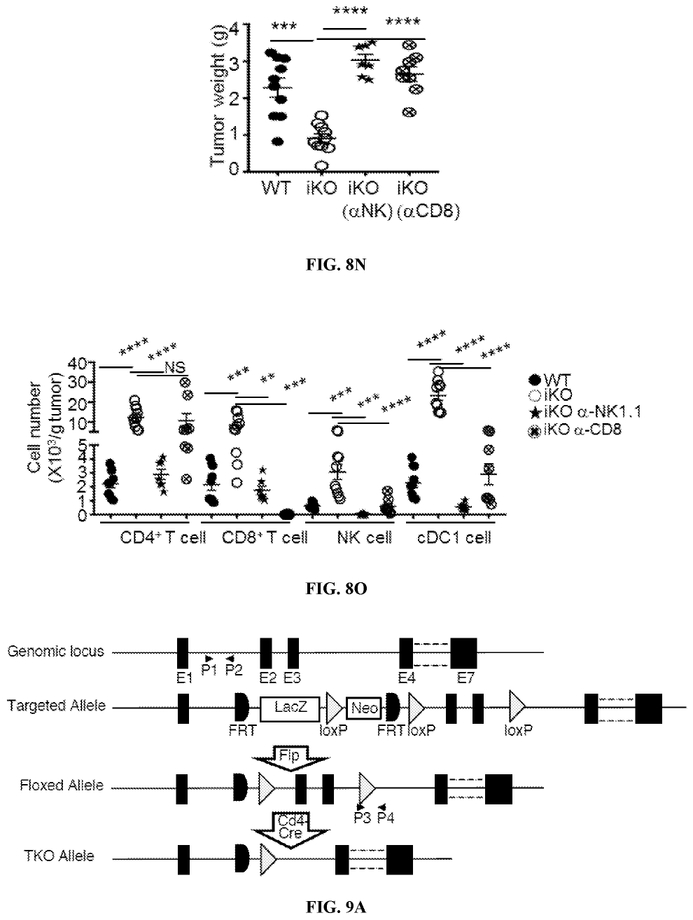

Although tolerance prevents autoimmunity, it poses a major obstacle to immune responses against cancer, and a general principle of cancer immunotherapy is to overcome immune tolerance (Maueroder et al., 2014). The finding that Otub1 controls the activation of CD8 T cells and NK cells, central components for cancer immunity (Durgeau et al., 2018; Chiossone et al., 2018), suggested a role for Otub1 in regulating antitumor immunity. The T cell-specific functions of Otub1 were first tested by employing the Otub1-TKO mice and a murine melanoma model, B16-OVA (B16 cells expressing the surrogate antigen ovalbumin). Compared with WT mice, Otub1-TKO mice had significantly reduced tumor burden (FIGS. 8A&B), coupled with increased frequencies of CD8 effector T cells producing IFN-γ and Granzyme B in both tumors and draining lymph nodes (FIG. 8C). Furthermore, the Otub1-TKO CD8 T cells expressed higher levels of Glut1 than WT CD8 T cells in tumor microenvironment (FIG. 8D), consistent with the role of Otub1 in regulating glycolysis (FIGS. 6C&D).

To examine the therapeutic potential of targeting Otub1, a mouse model of adoptive T cell therapy (ACT) was employed (Restifo et al., 2012). B6 mice were inoculated with B16F10 melanoma cells and then the tumor-bearing mice were treated by adoptive transfer of in vitro expanded CD8 T cells derived from WT or Otub1-TKO Pmel1 mice (FIG. 8E). The Pmel1 CD8 T cells recognize the tumor antigen gp100 expressed by B16F10 tumors. Compared to the WT Pmel1 CD8 T cells, the Otub1-TKO Pmel1 CD8 T cells were profoundly more effective in suppressing tumor growth and improving survival of the B16 tumor-bearing mice (FIGS. 8F&G).

Next, the Otub1-iKO model, in which Otub1 was inducibly deleted in adult mice in different cell types, was challenged with B16F10 tumor cells (FIG. 8H). The Otub1-iKO mice had greatly reduced tumor burden compared to WT mice (FIGS. 8I&J), associated with increased tumor-infiltrating CD8 T cells and NK cells as well as CD4 T cells and cDC1 cells (FIG. 8K). Moreover, tumor-infiltrating CD8 T cells in the Otub1-iKO mice contained a significantly higher frequency of effector cells expressing IFN-γ and Granzyme B (FIG. 8L). Similar results were obtained with the MC38 colon cancer model (FIGS. 14A-C). Antibody-mediated depletion of either CD8 T cells or NK cells impaired the potent anticancer immunity of Otub1-iKO mice, causing the increase of tumor burden to a level similar to or higher than that in WT mice (FIGS. 8M&N, FIGS. 14D&E). NK cell depletion in Otub1-iKO mice drastically reduced the tumor-infiltrating cDC1 and CD4 T cells, whereas CD8 T cell depletion partially reduced the tumor-infiltrating cDC1, but not CD4 T cells (FIG. 8O). These results suggest that hyper-activation of CD8 T cells and NK cells contributes to the strong anticancer immunity in the Otub1-iKO mice.

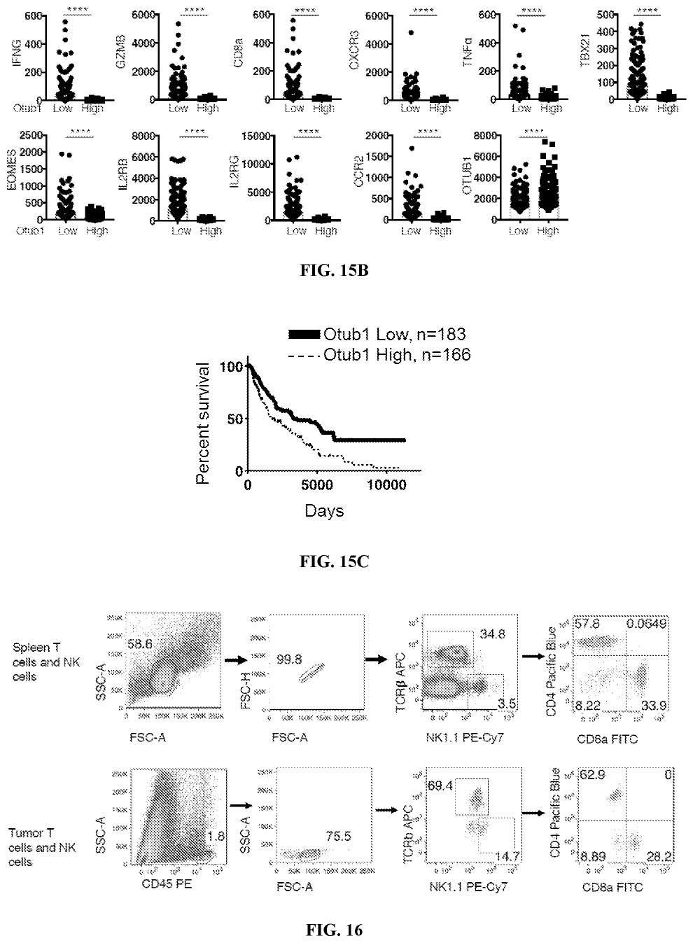

To assess the role of Otub1 in regulating antitumor immunity in human cancers, cancer databases were analyzed for potential correlation of Otub1 expression with T cell gene signature in tumors. Interestingly, an analysis of human skin cutaneous melanoma databases revealed a remarkable inverse correlation between Otub1 expression levels and the abundance of CD8 effector T cell gene signature as well as patient survival (FIGS. 15A-C). Collectively, these results establish Otub1 as an important regulator of antitumor immunity and implicate Otub1 as a potential target for cancer immunotherapy.

### Example 10—Otub1 Modulation Improves CAR Immunotherapy

Immunotherapy has become a promising therapeutic strategy for the treatment to many types of cancer. Among the major approaches of cancer immunotherapy are (1) targeting immune checkpoint receptors, such as programmed cell death protein (PD-1) and cytotoxic T cell lymphocyte-associated protein (CTLA-4) (Pardoll, 2012) and (2) and adoptive cell therapy using T cells expressing chimeric antigen receptors (CARs) that recognize tumor-associated antigens (Kuwana et al., 1987). CAR T cell immunotherapies have shown promise in the treatment of B cell malignancies (Maude et al., 2014). However, despite the attempts to modify the signaling motifs of the CARs, the efficacy of this approach for treating solid tumors is still low partly because of functional exhaustion of CAR T cells in tumor microenvironment (Wherry, 2011; Schietinger et al., 2016; Seo et al., 2019). Therapies based on a combination of checkpoint-blocking antibodies and CAR T cells have been tested in clinical trials hoping to invigorate the exhausted CAR T cells (Ramello et al., 2018). With a better understanding of the signaling mechanisms regulating T cell activation and exhaustion, it has become a valid approach to engineer functionally improved CAR T cells by targeting intracellular signaling regulators.

The concept of designing CARs is to link an extracellular single-chain variable fragment (ScFV) to an intracellular signaling module that includes signaling domains from CD3z, the costimulatory receptor CD28, and other costimulatory molecules, to induce T cell activation upon antigen binding (Srivastava and Riddell, 2015). Such a designing strategy is based on the fact that T cell activation requires both the TCR signal (signal 1) and costimulatory signals (signal 2). However, it is now clear that optimal T cell activation and effector function require additional signals, such as environmental cues (Curtsinger and Mescher, 2010). In particular, T cells receive signals from specific cytokines (signal 3) both during their priming in lymphoid organs and their effector functions in cancer microenvironments (Curtsinger et al., 1999). One important immunostimulatory cytokine is IL-15, which mediates the homeostasis, activation, and survival of CD8 T cells as well as natural killer (NK) cells and has been implicated in the regulation of antitumor immunity (Klebanoff et al., 2004). Scarcity of IL-15 signals in tumor site has been linked to poor cancer regression (Santana Carrero et al., 2019).

As shown above, loss of Otub1 dramatically enhances IL-15 mediated CD8 T cell and NK cell activation and anti-tumor immunity through increasing immune cell recruitment to tumor site and cytokine secretion (Zhou et al., 2019). However, it has been unclear whether targeting Otub1 can be used as an approach to improve the function of CAR T and CAR NK cells in cancer immunotherapy. Here, a mouse model of CAR immunotherapy was used to demonstrate that Otub1 knockout or knockdown in CAR-transduced CD8 T cells or NK cells markedly enhances the efficacy of immunotherapy against solid tumors.

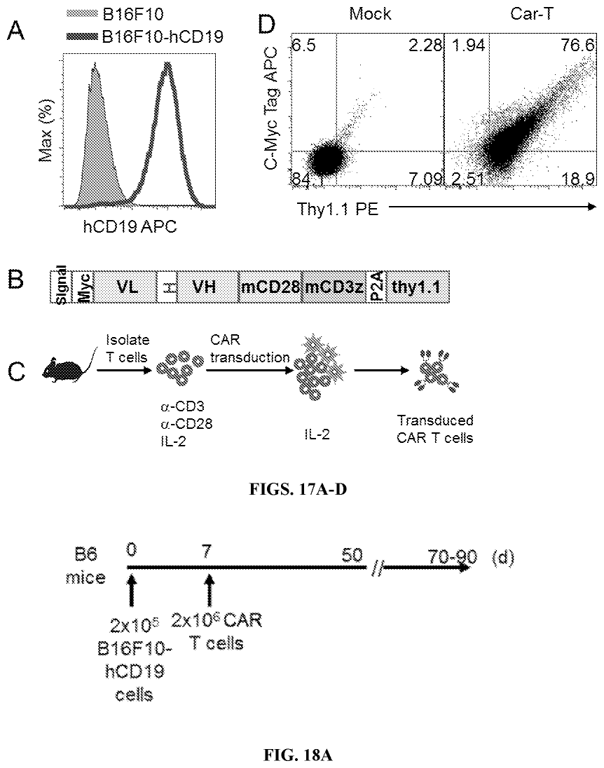

To examine the therapeutic potential of targeting Otub1 for improving the efficacy of CAR T cell-mediated solid tumor treatment, a preclinical model allowing in vivo assays of tumor rejection was set up. Briefly, a mouse B16F10 cell line was engineered to stably express the human B cell-specific antigen hCD19 (B16F10-hCD19) and the expression of hCD19 was confirmed by flow cytometry (FIG. 17A). Then, a second-generation CAR was constructed against hCD19 (anti-hCD19 CAR) and transduced into in vitro activated mouse CD8 T cells (FIGS. 17B,C). Flow cytometry assays, based on expression of Myc epitope-tagged CAR and mouse thy1.1, revealed a high efficiency of transduction (FIG. 17D).

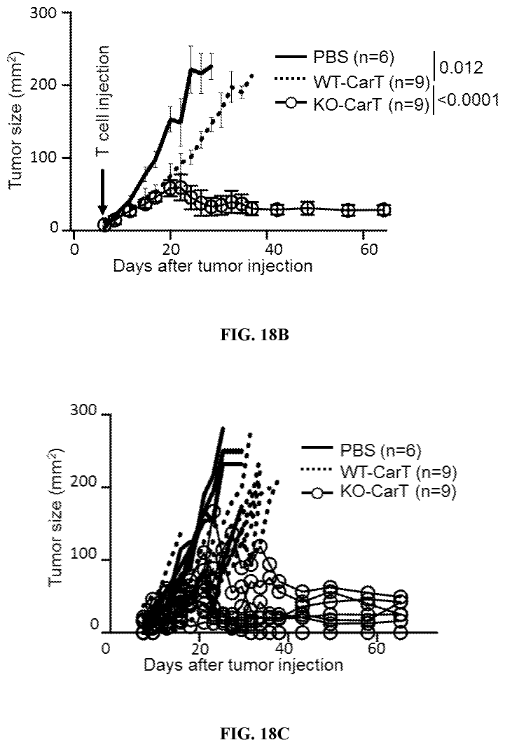

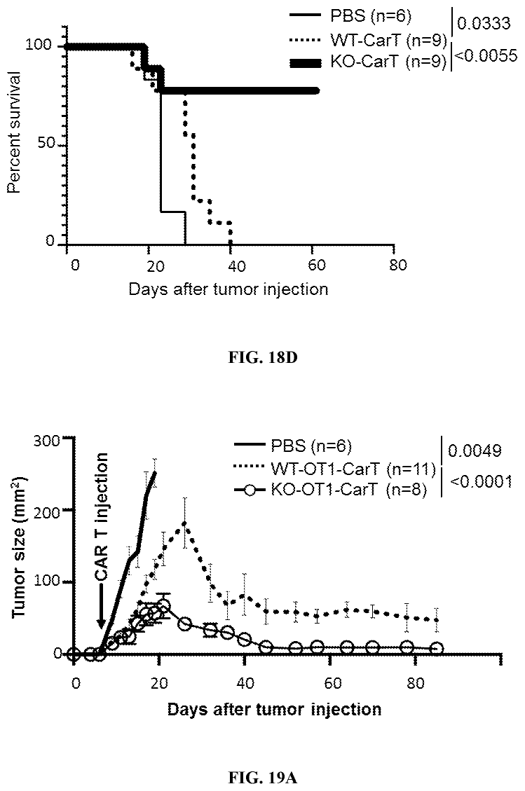

For CAR T cell-mediated therapy, B6 mice were inoculated with B16F10-hCD19 melanoma cells, and then the tumor-bearing mice were treated by adoptively transferring in vitro-expanded CD8 anti-hCD19 CAR T cells derived from WT or Otub1 T cell-conditional knockout (Otub1-TKO) mice (FIG. 18A). The anti-hCD19 CAR CD8 T cells recognize the hCD19 antigen overexpressed by B16F10-hCD19 tumors, allowing antigen-specific tumor cell destruction. Compared to control mice injected with phosphate-buffered saline (PBS), mice transferred with anti-hCD19 CAR WT T cells had moderately reduced tumor size (FIGS. 18B,C). Importantly, transfer of anti-CD19 CAR Otub1-TKO T cells caused a much more profound suppression of tumor growth, coupled with a drastically improved survival rate of the B16F10-hCD19 tumor-bearing mice (FIGS. 18B-D).

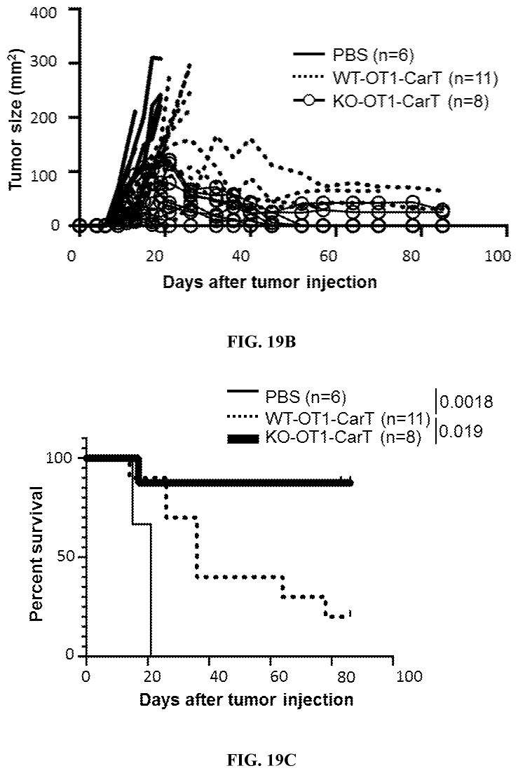

Since CARs employ a fixed artificial scFv to bypass the need of traditional T cell receptor (TCR) for antigen activation (Srivastava and Riddell, 2015), TCR transgenic CD8 T cells might be a replacement of polyclonal CD8 T cells. To test this idea, CAR T cells were generated by using CD8 T cells derived from OT-I mice, which produce CD8 T cells with recombinant TCR specific for the chicken oval-albumin (OVA) peptide SIINFEKL (Hogquist et al., 1994). The anti-CD19 CAR-transduced WT or Otub1-TKO OT-I T cells were adoptively transferred into B16F10-hCD19 tumor bearing mice followed by measuring tumor growth and survival rate. Interestingly, in this model, the WT anti-hCD19 CAR T cells showed a strong tumor-suppressing function (FIGS. 19A,B). Nevertheless, as seen with the polyclonal CD8 T cell model, mice treated with the Otub1-TKO anti-hCD19 CAR OT-I T cells displayed a much stronger tumor suppression and improved survival than those treated with the WT anti-hCD19 CAR OT1 T cells (FIGS. 19A-C). These results show that Otub1 deletion greatly improves the function of CAR T cells in tumor suppression.

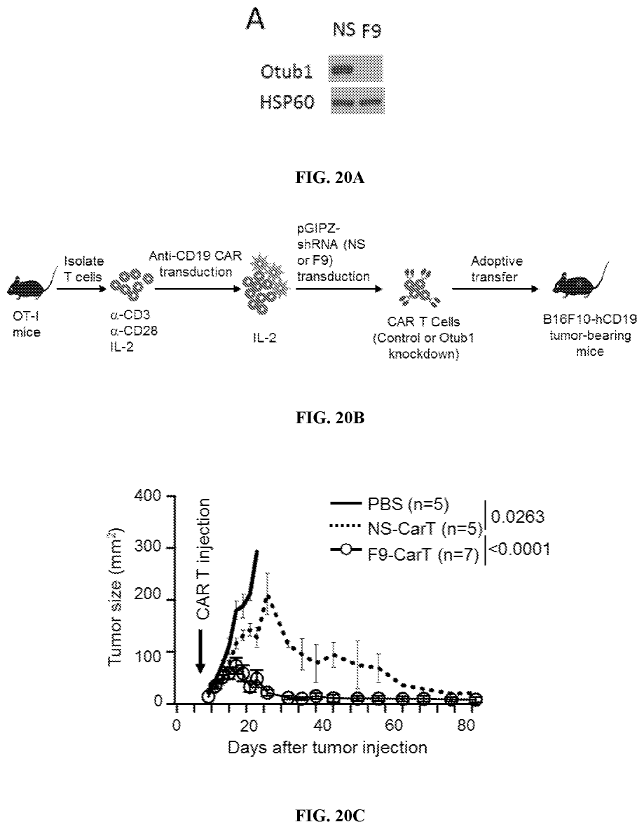

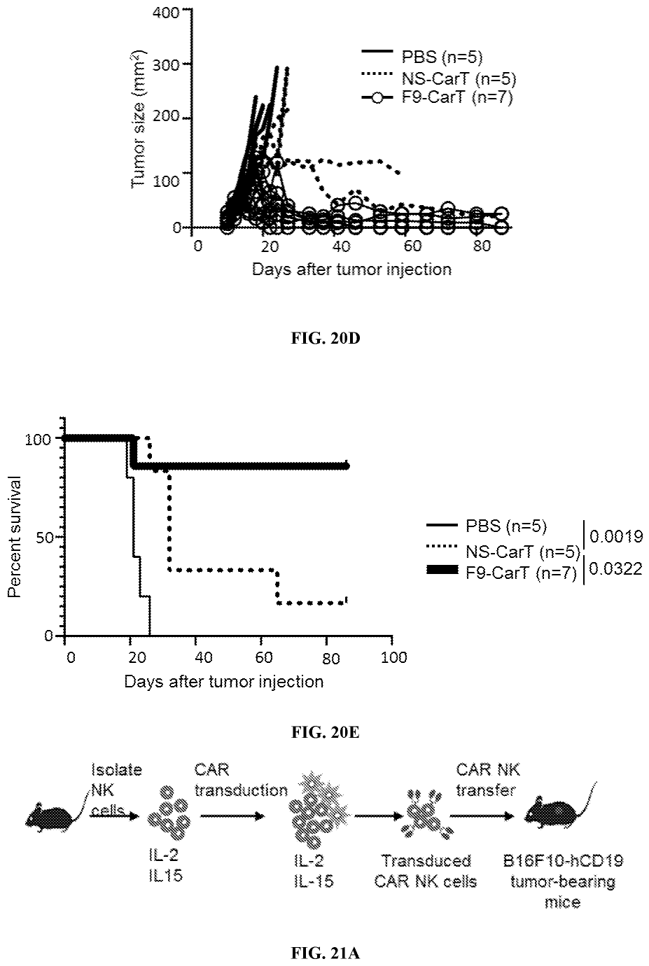

RNA interference represents a promising therapeutic strategy in cancer immunotherapy through silencing specific target genes (Ghafouri-Fard and Ghafouri-Fard, 2012). To further translate these findings into clinically applicable concepts, it was examined whether Otub1 knockdown by short hairpin RNAs (shRNAs) could improve the efficacy of CAR T cell-mediated tumor suppression. A mouse Otub1-specific shRNA (F9), cloned into the into pGIPZ lentiviral vector, could efficiently silence the expression of Otub1 (FIG. 20A). WT OT-I CD8 T cells were transduced with a non-silencing (NS) control shRNA or the Otub1-specific shRNA F9, and the cells were further transduced with anti-CD19 CAR to generate control (NS-CarT) and Otub1 knockdown (F9-CarT) CAR T cells, respectively (FIG. 20B). Adoptive transfer NS-CarT into B16F10-hCD19 tumor-bearing B6 mice suppressed tumor growth as compared with injection with PBS; however, adoptive transfer of F9-CarT caused much more profound tumor suppression than the transfer of NS-CarT cells (FIGS. 20C,D). Consistently, the tumor-bearing mice treated with F9-CarT cells had significantly improved survival rate compared to those treated with the NS-CarT cells (FIG. 20E). These data emphasized the potential of silencing Otub1 for cancer immunotherapy based on adoptive CAR T cell transfer. For Otub1 knockdown in human T cells, several novel shRNAs targeting human Otub1 were designed and cloned into the pGIPZ lentiviral vector (FIG. 22A). By transducing human 293T cell line, it was found that two of these newly designed shRNAs were efficient in silencing human Otub1 (FIG. 22B).

In addition to CD8 T cells, NK cells are an important part of the cellular immune system, with a potent ability to kill tumor and virally infected cells. NK cells mediate their tumor-killing function without requiring MHC matching, making them an ideal candidate to generate “off-the-shelf” universal CAR products for large-scale clinical applications (Ruella and Kenderian, 2017).

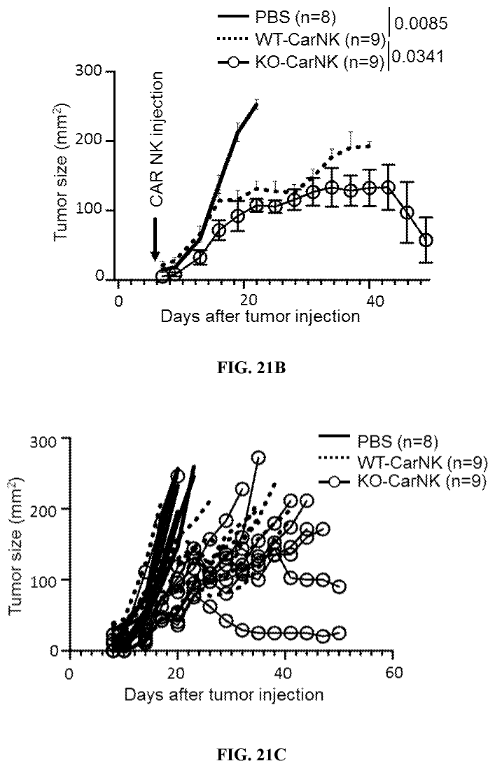

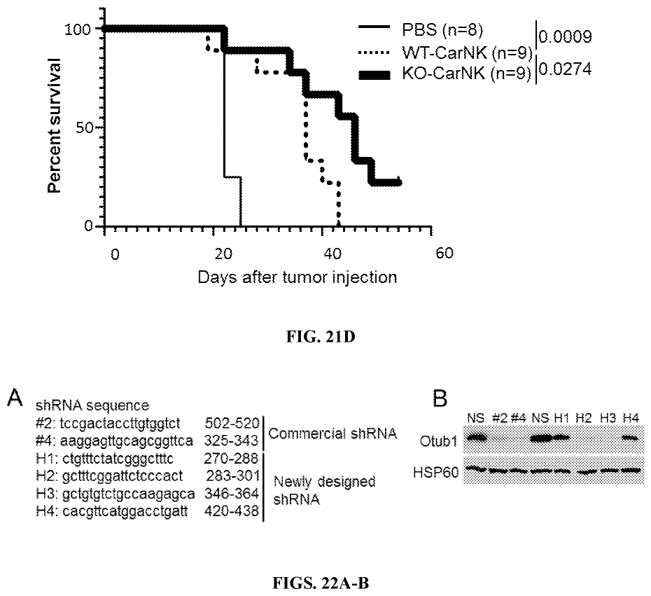

To test the effect of targeting Otub1 on CAR NK cell-mediated anti-tumor immunity, Otub1 was inducibly deleted in adult mice with a tamoxifen-inducible Cre (CreER) system. NK cells isolated from WT or induced Otub1 knockout (Otub1-iKO) mice were in vitro transduced with the anti-CD19 CAR construct. B16F10-hCD19 tumor bearing mice were then adoptively transferred with 3×10⁶ WT or Otub1-iKO CAR NK cells on day 7. As seen with CAR T transfer, adoptive transfer of CAR NK cells resulted in strong suppression of tumor growth (FIGS. 21B,C). Moreover, compared to the WT CAR NK cells, the Otub1-deficient CAR NK cells were significantly more efficient in suppressing tumor growth and improving the survival rate of the tumor-bearing mice (FIGS. 21B-D). Thus, targeting Otub1 may be an effective approach to improve the efficacy of cancer immunotherapies based on adoptive transfer of both CAR T cells and CAR NK cells.

The results present here suggest that targeting Otub1 enhances CAR T and CAR NK cell-based immunotherapies for treating solid tumors. The potency of CAR-mediated tumor cell targeting can be combined with the ability of boosting IL-15 signaling to enhance the function of CAR T and CAR NK cells. IL-15 has been widely considered a promising cancer immunotherapy agent for more than a decade; however, clinical trials based on recombinant IL-15 injection have not shown promising results due to several limitations, such as short half-life and necessity for using high doses that cause toxicity (Robinson and Schluns, 2017). More recent studies suggest that IL-15 is produced in tumor microenvironment, but the level of IL-15 is low in advanced tumors (Santana Carrero et al., 2019). Innate immune stimuli, such as STING agonists, can stimulate IL-15 production, thus contributing to the induction of antitumor immunity (Santana Carrero et al., 2019). Otub1 is a pivotal negative regulator of IL-15 induced signaling (Zhou et al., 2019). Importantly, deletion of Otub1 in adult mice is sufficient for triggering endogenous IL-15 signaling in CD8 T cells and NK cells, causing drastically enhanced antitumor immunity (Zhou et al., 2019). Data from the present study further demonstrate that targeting Otub1 is an effective approach to boost the antitumor functions of CAR T cells and CAR NK cells in adoptive cell therapy.

One major challenge of CAR-T cell therapy is its limited efficacy in treating solid tumors (Martinez and Moon, 2019). Among the major factors that limit the function of CAR T cells is the immunosuppressive tumor microenvironment rendering CAR T cells hypofunctional (Wherry, 2011). Thus, modulating the signaling network in CAR T cells to boost their functions represents a new strategy for improving the efficacy of CAR T cell-mediated solid tumor therapy. Given the potent immunostimulatory function of IL-15 and its production in tumor microenvironment, manipulating IL-15 signaling pathway represents an attractive strategy. In line with previous findings that Otub1 deficiency greatly sensitizes CD8 T cells to IL-15 stimulation, the present study demonstrated that genetic ablation or shRNA-mediated knockdown of Otub1 profoundly promote the function of CAR T cells in suppressing B16 melanoma growth and extending the survival of tumor-bearing mice. In addition to CD8 T cells, NK cells serve as a target of IL-15 and rely on IL-15 for survival and activation. Consistently, the data revealed that Otub1 deletion also enhanced the antitumor function of CAR NK cells. One advantage of CAR NK cells is their lack of activity to induce graft-versus-host disease even in MHC-mismatched patients, thus providing a potential source of “off-the-shelf” therapeutic tool (Ruella and Kenderian, 2017). To date, most CAR NK studies, including the present study, use CARs designed for T cells that are not optimized for NK cell signaling (Li et al., 2018). Even so, CAR NK cells with Otub1 knockout still showed markedly enhanced ability to mediate tumor regression. It is reasonable to expect more significant reduction of tumor size with NK cell-optimized CARs. Taken together, these findings have important implications for cancer immunotherapy, since CD8 T cells and NK cells are two primary and functionally complementary cellular components in cancer immunity.

### Example 11—Materials and Methods for Example 10

Mice. Otub1fl/fl mice, described previously (Zhou et al., 2019) were crossed with CD4-Cre transgenic mice (on B6 genetic background and from Jackson laboratories) to produce age-matched Otub1⁺/+CD4-Cre (named WT) and Otub1fl/flCD4-Cre (named T cell-conditional Otub1 knockout or TKO) mice. The Otub1fl/fl mice were also crossed with ROSA26-CreER (Jackson Laboratories) to generate Otub1⁺/+ROSA26-CreER and Otub1fl/flROSA26-CreER mice, which were then injected intraperitoneally with tamoxifen (2 mg per mouse) in corn oil daily for four consecutive days to induce Cre function for generating WT and induced KO (iKO) mice, respectively. OT-I TCR-transgenic mice and B6 mice were from Jackson Laboratories. Experiments were performed with young adult (6- to 8-week-old) female and male mice except where indicated otherwise. All mice were on the B6 genetic background and maintained in a specific-pathogen-free facility of the university of Texas MD Anderson Cancer Center, and all animal experiments were done in accordance with protocols approved by the Institutional Animal Care and Use Committee of the University of Texas MD Anderson Cancer Center.

Cell lines, plasmids, antibodies and reagents. Human HIKE293T and murine EL4 and B16F10 cell lines were from ATCC. hCD19 in lentiviral vector PLOC (precision lentiORF expression library) was purchased from the Functional Genomics Core of MD Anderson Cancer Center. Functional-grade anti-mouse (m) CD3e (145-2C11) and anti-mCD28 (27.51) were from eBioscience. Fluorescently labeled antibodies for mThy1.1 was from BD Biosciences. Myc-Tag (9B111) was from cell signaling. Recombinant mIL-2 and mIL-15 cytokines were from R&D. Anti-mCD8a- and anti-mThy1.1-conjugated microbeads and mouse NK cell isolation kit were from Miltenyi.

Construction of anti-hCD19 CAR. Anti-hCD19 CAR was designed by using published segment of the clone FMC63 of anti-hCD19 single chain variable fragment (Nicholson et al., 1997), with a portion of murine CD28 and CD3z sequences (Kochenderfer et al., 2010). The sequence for Myc-tag and hCD8 signal peptide at the N terminus was obtained from public database. The complete CAR construct was synthesized by Twist Bioscience then sub-cloned into the pMGIR1 murine retroviral vector containing the internal ribosome entry site (IRES)-EGFP reporter gene for cell selection.

Nucleotide sequence of CAR construct with Thy1.1 (hCD8 signal-Myc-VL-hinge-VH-mCD28-mCD3z-P2A-thy1.1:(2016 nt)

(SEQ ID NO: 7)
atggCTTTGCCAGTGACAGCTCTTCTCCTTCCACTGGCCCTCCTCCTTCAC
GCCGCTAGGCCAGAGCAGAAACTTATTTCAGAGGAAGACCTGGACATTCAA
ATGACACAAACTACTTCTTCTCTCTCCGCCTCACTTGGTGACCGCGTCACT
ATTAGTTGCCGCGCTAGTCAAGATATTAGTAAGTACCTGAATTGGTATCAA
CAAAAACCTGACGGGACTGTAAAGCTGCTTATATATCATACTTCTAGGCTG
CATTCTGGAGTACCTTCACGATTTAGCGGTAGCGGATCCGGCACCGACTAC
TCCCTCACAATTAGCAATCTGGAGCAAGAGGACATAGCCACCTACTTCTGC
CAGCAAGGGAATACCTTGCCATACACTTTCGGTGGTGGAACTAAGCTCGAA
ATTACTGGGGGTGGAGGCAGTGGCGGAGGGGGGTCAGGTGGGGGAGGTTCA
GAAGTCAAACTCCAGGAATCTGGACCTGGACTCGTTGCCCCTTCCCAATCC
CTTAGTGTTACATGCACTGTATCAGGTGTATCCCTCCCTGATTACGGTGTC
TCCTGGATTCGGCAGCCTCCTCGGAAGGGTCTCGAGTGGTTGGGAGTGATT
TGGGGGTCTGAAACTACTTATTATAACAGTGCCCTTAAGAGTAGATTGACT
ATAATTAAGGATAACAGTAAGTCACAAGTATTCCTCAAAATGAATTCCTTG
CAAACAGACGATACAGCAATATATTACTGCGCAAAACACTACTACTATGGC
GGTAGTTACGCTATGGACTATTGGGGTCAAGGAACCTCTGTCACAGTTTCT
AGCATTGAGTTCATGTATCCCCCACCTTACTTGGACAATGAAAGGTCTAAT
GGGACCATCATACACATTAAAGAGAAACACCTGTGTCATACTCAGAGTTCT
CCAAAATTGTTCTGGGCCTTGGTTGTCGTTGCCGGCGTACTGTTCTGTTAC
GGTCTCTTGGTTACCGTGGCACTTTGTGTTATCTGGACTAATTCCCGGCGG
AATCGGGGTGGACAGAGCGATTACATGAATATGACCCCAAGAAGACCTGGA
CTGACCAGGAAACCATATCAACCCTATGCTCCTGCTCGGGACTTTGCTGCT
TACCGCCCACGCGCAAAGTTTTCTAGGAGCGCTGAAACCGCTGCCAACCTC
CAAGACCCTAATCAGCTTTACAATGAATTGAACTTGGGACGCCGGGAGGAG
TATGACGTCCTTGAGAAAAAGCGGGCTCGGGATCCAGAAATGGGCGGAAAG
CAACAGAGGCGAAGAAATCCACAAGAGGGGGTCTATAACGCTCTTCAGAAA
GATAAAATGGCTGAGGCATATAGCGAAATTGGGACCAAGGGGGAGAGAAGA
AGAGGCAAGGGACATGACGGGCTTTACCAGGGTTTGTCTACCGCAACAAAA
GACACCTATGATGCTTTGCACATGCAAACACTGGCTCCTAGAGCCACCAAC
TTCTCCCTGCTGAAGCAGGCCGGCGACGTGGAGGAGAACCCCGGCCCCATG
AACCCAGCCATCAGCGTCGCTCTCCTGCTCTCAGTCTTGCAGGTGTCCCGA
GGGCAGAAGGTGACCAGCCTGACAGCCTGCCTGGTGAACCAAAACCTTCGC
CTGGACTGCCGCCATGAGAATAACACCAAGGATAACTCCATCCAGCATGAG
TTCAGCCTGACCCGAGAGAAGAGGAAGCACGTGCTCTCAGGCACCCTCGGG
ATACCCGAGCACACGTACCGCTCCCGCGTCACCCTCTCCAACCAGCCCTAT
ATCAAGGTCCTTACCCTAGCCAACTTCACCACCAAGGATGAGGGCGACTAC
TTTTGTGAGCTTCGAGTCTCGGGCGCGAATCCCATGAGCTCCAATAAAAGT
ATCAGTGTGTATAGAGACAAACTGGTCAAGTGTGGCGGCATAAGCCTGCTG
GTTCAGAACACATCCTGGATGCTGCTGCTGCTGCTTTCCCTCTCCCTCCTC
CAAGCCCTGGACTTCATTTCTCTGTGA

Amino acid sequence of CAR construct plus Thy1.1: (671AA)

(SEQ ID NO: 8)
MALPVTALLLPLALLLHAARPEQKLISEEDLDIQMTQTTSSLSASLGDRVT
ISCRASQDISKYLNWYQQKPDGTVKLLIYHTSRLHSGVPSRFSGSGSGTDY
SLTISNLEQEDIATYFCQQGNTLPYTFGGGTKLEITGGGGSGGGGSGGGGS
EVKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYYG
GSYAMDYWGQGTSVTVSSIEFMYPPPYLDNERSNGTIIHIKEKHLCHTQSS
PKLFWALVVVAGVLFCYGLLVTVALCVIWTNSRRNRGGQSDYMNMTPRRPG
LTRKPYQPYAPARDFAAYRPRAKFSRSAETAANLQDPNQLYNELNLGRREE
YDVLEKKRARDPEMGGKQQRRRNPQEGVYNALQKDKMAEAYSEIGTKGERR
RGKGHDGLYQGLSTATKDTYDALHMQTLAPRATNFSLLKQAGDVEENPGPM
NPAISVALLLSVLQVSRGQKVTSLTACLVNQNLRLDCRHENNTKDNSIQHE
FSLTREKRKHVLSGTLGIPEHTYRSRVTLSNQPYIKVLTLANFTTKDEGDY
FCELRVSGANPMSSNKSISVYRDKLVKCGGISLLVQNTSWMLLLLLSLSLL
QALDFISL*

Retroviral and lentiviral infections. Retroviral particles were prepared using the pMIGR1-CAR expression vectors along with the packaging vector pCL-ECO, as previously described (Zhou et al., 2019). For production of lentiviral particles, HTEK293T cells were transfected (by PEI method) with PLOC lentiviral vector encoding hCD19 or pGIPZ lentiviral vectors encoding Otub1-specific shRNAs or a non-silencing control shRNA along with the packaging vectors psPAX2 and pMD2. CD8 T cells, NK cells and B16F10 melanoma cells were infected with the recombinant retroviruses or lentiviruses. After 48 h, the transduced cells were enriched by flow cytometric cell sorting based on GFP expression. CAR T and CAR NK cells were purified by using anti-Thy1.1 microbeads.

Statistical analysis. For tumor clinical scores, differences between groups were evaluated by two-way ANOVA with Bonferroni correlation. For survival. Differences between groups were evaluated by log-rank test. P values less than 0.05 were considered significant. All statistical tests were justified as appropriate and the data met the assumptions of the tests. The variance was similar between the groups being statistically compared.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Balakirev, M. Y., Tcherniuk, S. O., Jaquinod, M. & Chroboczek, J. Otubains: a new family of cysteine proteases in the ubiquitin pathway. EMBO Rep. 4, 517-522 (2003).
Bottcher, J. P. et al. N K Cells Stimulate Recruitment of cDC1 into the Tumor Microenvironment Promoting Cancer Immune Control. Cell 172, 1022-1037 e1014 (2018).
Burkett, P. R. et al. IL-15R alpha expression on CD8⁺ T cells is dispensable for T cell memory. Proc Natl Acad Sci USA 100, 4724-4729 (2003).
Calleja, V., Laguerre, M., Parker, P. J. & Larijani, B. Role of a novel PH-kinase domain interface in PKB/Akt regulation: structural mechanism for allosteric inhibition. PLoS Biol 7, e17 (2009).
Cammann, C. et al. Early changes in the metabolic profile of activated CD8(+) T cells. BMC Cell Biol 17, 28 (2016).
Cantley, L. C. The phosphoinositide 3-kinase pathway. Science 296, 1655-1657 (2002).
Carnero, A., Blanco-Aparicio, C., Renner, O., Link, W. & Leal, J. F. The PTEN/PI3K/AKT signalling pathway in cancer, therapeutic implications. Curr Cancer Drug Targets 8, 187-198 (2008).
Castillo, E. F. & Schluns, K. S. Regulating the immune system via IL-15 transpresentation. Cytokine 59, 479-490 (2012).
Chiossone, L. et al. Maturation of mouse NK cells is a 4-stage developmental program. Blood 113, 5488-5496 (2009).
Chiossone, L., Dumas, P. Y., Vienne, M. & Vivier, E. Natural killer cells and other innate lymphoid cells in cancer. Nat Rev Immunol 18, 671-688 (2018).
Crouse, J., Xu, H. C., Lang, P. A. & Oxenius, A. NK cells regulating T cell responses: mechanisms and outcome. Trends Immunol 36, 49-58 (2015).
Curtsinger, J. M. and M. F. Mescher, Inflammatory cytokines as a third signal for T cell activation. Curr Opin Immunol, 2010. 22(3): p. 333-40.
Curtsinger, J. M., et al., Inflammatory cytokines provide a third signal for activation of naive CD4+ and CD8+ T cells. J Immunol, 1999. 162(6): p. 3256-62.
Deshpande, P. et al. IL-7- and IL-15-mediated TCR sensitization enables T cell responses to self-antigens. J Immunol 190, 1416-1423 (2013).
Dubois, S., Mariner, J., Waldmann, T. A. & Tagaya, Y. IL-15Ralpha recycles and presents IL-15 In trans to neighboring cells. Immunity 17, 537-547 (2002).
Durgeau, A., Virk, Y., Corgnac, S. & Mami-Chouaib, F. Recent Advances in Targeting CD8 T-Cell Immunity for More Effective Cancer Immunotherapy. Front Immunol 9, 14 (2018).
Ghafouri-Fard, S. and S. Ghafouri-Fard, siRNA and cancer immunotherapy. Immunotherapy, 2012. 4(9): p. 907-17.
Goldrath, A. W. et al. Cytokine requirements for acute and Basal homeostatic proliferation of naive and memory CD8⁺ T cells. J Exp Med 195, 1515-1522 (2002).
Gubser, P. M. et al. Rapid effector function of memory CD8⁺ T cells requires an immediate-early glycolytic switch. Nat Immunol 14, 1064-1072 (2013).
Guillerey, C., Huntington, N. D. & Smyth, M. J. Targeting natural killer cells in cancer immunotherapy. Nat Immunol 17, 1025-1036 (2016).
Hogquist, K. A. et al. T cell receptor antagonist peptides induce positive selection. Cell 76, 17-27 (1994).
Hu, H. & Sun, S. C. Ubiquitin signaling in immune responses. Cell Res. 26, 457-483 (2016).
Huang, P. L. et al. Skeletal muscle interleukin 15 promotes CD8(+) T-cell function and autoimmune myositis. Skelet Muscle 5, 33 (2015).
Jethwa, N. et al. Endomembrane PtdIns(3,4,5)P3 activates the PI3K-Akt pathway. J Cell Sci 128, 3456-3465 (2015).
Juang, Y. C. et al. OTUB1 co-opts Lys48-linked ubiquitin recognition to suppress E2 enzyme function. Mol Cell 45, 384-397 (2012).
Kim, E. H. et al. Signal integration by Akt regulates CD8 T cell effector and memory differentiation. J Immunol 188, 4305-4314 (2012).
Kim, E. H. & Suresh, M. Role of PI3K/Akt signaling in memory CD8 T cell differentiation. Front Immunol 4, 20 (2013).
Klebanoff, C. A. et al. IL-15 enhances the in vivo antitumor activity of tumor-reactive CD8+ T cells. Proc Natl Acad Sci USA 101, 1969-1974 (2004).
Kochenderfer et al. Adoptive transfer of syngeneic T cells transduced with a chimeric antigen receptor that recognizes murine CD19 can eradicate lymphoma and normal B cells. Blood, 116:3875-3886 (2010).
Kuwana, Y., et al., Expression of chimeric receptor composed of immunoglobulin-derived V regions and T-cell receptor-derived C regions. Biochem Biophys Res Commun, 1987. 149(3): p. 960-8.
Li et al. Human iPSC-Derived Natural Killer Cells Engineered with Chimeric Antigen Receptors Enhance Anti-tumor Activity. Cell Stem Cell, 23:181-192 e5 (2018).
Liu, K., Catalfamo, M., Li, Y., Henkart, P. A. & Weng, N. P. IL-15 mimics T cell receptor crosslinking in the induction of cellular proliferation, gene expression, and cytotoxicity in CD8+ memory T cells. Proc Natl Acad Sci USA 99, 6192-6197 (2002).
Lodolce, J. P., Burkett, P. R., Koka, R. M., Boone, D. L. & Ma, A. Regulation of lymphoid homeostasis by interleukin-15. Cytokine Growth Factor Rev 13, 429-439 (2002).
Martinez and Moon, CAR T Cells for Solid Tumors: New Strategies for Finding, Infiltrating, and Surviving in the Tumor Microenvironment. Front Immunol, 10, 128 (2019).
Maude, S. L., et al., Chimeric antigen receptor T cells for sustained remissions in leukemia. N Engl J Med, 2014. 371(16): p. 1507-17.
Maueroder, C. et al. Tumor Immunotherapy: Lessons from Autoimmunity. Frontiers in immunology 5, 212 (2014).
McKinney, E. F. & Smith, K. G. C. Metabolic exhaustion in infection, cancer and autoimmunity. Nat Immunol 19, 213-221 (2018).
Mishra, A., Sullivan, L. & Caligiuri, M. A. Molecular pathways: interleukin-15 signaling in health and in cancer. Clin Cancer Res 20, 2044-2050 (2014).
Nakada, S. et al. Non-canonical inhibition of DNA damage-dependent ubiquitination by OTUB1. Nature 466, 941-946 (2010).
Nicholson et al. Construction and characterisation of a functional CD19 specific single chain Fv fragment for immunotherapy of B lineage leukaemia and lymphoma. Mol Immunol, 34, 1157-1165 (1997).
Overwijk, W. W. et al. Tumor regression and autoimmunity after reversal of a functionally tolerant state of self-reactive CD8+ T cells. J Exp Med 198, 569-580 (2003).
Pardoll, The blockade of immune checkpoints in cancer immunotherapy. Nat Rev Cancer, 12, 252-264 (2012).
Pearce, E. L. & Shen, H. Generation of CD8 T cell memory is regulated by IL-12. J Immunol 179, 2074-2081 (2007).
Pearce, E. L., Poffenberger, M. C., Chang, C. H. & Jones, R. G. Fueling immunity: insights into metabolism and lymphocyte function. Science 342, 1242454 (2013).
Polansky, J. K. et al. High dose CD11c-driven IL15 is sufficient to drive NK cell maturation and anti-tumor activity in a trans-presentation independent manner. Sci Rep 6, 19699 (2016).
Ramello, M. C., E. B. Haura, and D. Abate-Daga, CAR-T cells and combination therapies: What's next in the immunotherapy revolution? Pharmacol Res, 2018. 129: p. 194-203.
Reiley, W. W. et al. Deubiquitinating enzyme CYLD negatively regulates the ubiquitin-dependent kinase Tak1 and prevents abnormal T cell responses. J. Exp. Med. 204, 1475-1485 (2007).
Restifo, N. P., Dudley, M. E. & Rosenberg, S. A. Adoptive immunotherapy for cancer: harnessing the T cell response. Nat Rev Immunol 12, 269-281 (2012).
Roberts, D. J. & Miyamoto, S. Hexokinase II integrates energy metabolism and cellular protection: Akting on mitochondria and TORCing to autophagy. Cell Death Differ 22, 248-257 (2015).
Robinson, T. O. and K. S. Schluns, The potential and promise of IL-15 in immuno-oncogenic therapies. Immunol Lett, 2017. 190: p. 159-168.
Rosenberg, J. & Huang, J. CD8(+) T Cells and NK Cells: Parallel and Complementary Soldiers of Immunotherapy. Curr Opin Chem Eng 19, 9-20 (2018).
Ruella, M. and S. S. Kenderian, Next-Generation Chimeric Antigen Receptor T-Cell Therapy: Going off the Shelf. BioDrugs, 2017. 31(6): p. 473-481.
Santana Carrero, R. M., et al., IL-15 is a component of the inflammatory milieu in the tumor microenvironment promoting antitumor responses. Proc Natl Acad Sci USA, 2019. 116(2): p. 599-608.
Schietinger, A., et al., Tumor-Specific T Cell Dysfunction Is a Dynamic Antigen-Driven Differentiation Program Initiated Early during Tumorigenesis. Immunity, 2016. 45(2): p. 389-401.
Schluns, K. S., Kieper, W. C., Jameson, S. C. & Lefrancois, L. Interleukin-7 mediates the homeostasis of naive and memory CD8 T cells in vivo. Nat Immunol 1, 426-432 (2000).
Schluns, K. S. & Lefrancois, L. Cytokine control of memory T-cell development and survival. Nat Rev Immunol 3, 269-279 (2003).
Schluns, K. S. et al. Distinct cell types control lymphoid subset development by means of IL-15 and IL-15 receptor alpha expression. Proc Natl Acad Sci USA 101, 5616-5621 (2004).
Seo, H., et al., TOX and TOX2 transcription factors cooperate with NR4A transcription factors to impose CD8(+) T cell exhaustion. Proc Natl Acad Sci USA, 2019. 116(25): p. 12410-12415.
Srivastava, S. and S. R. Riddell, Engineering CAR-T cells: Design concepts. Trends Immunol, 2015. 36(8): p. 494-502.
Sun, S. C. Deubiquitylation and regulation of the immune response. Nat. Rev. Immunol. 8, 501-511 (2008).
Surh, C. D. & Sprent, J. Homeostasis of naive and memory T cells. Immunity 29, 848-862 (2008).
Teague, R. M. et al. Interleukin-15 rescues tolerant CD8+ T cells for use in adoptive immunotherapy of established tumors. Nat Med 12, 335-341 (2006).
Vadlakonda, L., Dash, A., Pasupuleti, M., Anil Kumar, K. & Reddanna, P. The Paradox of Akt-mTOR Interactions. Front Oncol 3, 165 (2013).
Waldmann, T. A. The shared and contrasting roles of IL2 and IL15 in the life and death of normal and neoplastic lymphocytes: implications for cancer therapy. Cancer Immunol Res 3, 219-227 (2015).
Wang, T. et al. Evidence for bidentate substrate binding as the basis for the K48 linkage specificity of otubain 1. J Mol Biol 386, 1011-1023 (2009).
Wherry, E. J., T cell exhaustion. Nat Immunol, 2011. 12(6): p. 492-9.
Wiener, R., Zhang, X., Wang, T. & Wolberger, C. The mechanism of OTUB1-mediated inhibition of ubiquitination. Nature 483, 618-622 (2012).
Xiao, G., Harhaj, E. W. & Sun, S. C. NF-kappaB-inducing kinase regulates the processing of NF-kappaB2 p100. Mol. Cell. 7, 401-409 (2001).
Yang, W. L. et al. The E3 ligase TRAF6 regulates Akt ubiquitination and activation. Science 325, 1134-1138 (2009).
Yu, J. et al. Regulation of T-cell activation and migration by the kinase TBK1 during neuroinflammation. Nat. Commun. 6, 6074 (2015).
Zhang, P., Cote, A. L., de Vries, V. C., Usherwood, E. J. & Turk, M. J. Induction of postsurgical tumor immunity and T-cell memory by a poorly immunogenic tumor. Cancer Res 67, 6468-6476 (2007).
Zheng, Y., Delgoffe, G. M., Meyer, C. F., Chan, W. & Powell, J. D. Anergic T cells are metabolically anergic. J Immunol 183, 6095-6101 (2009).
Zhou, X., et al., The deubiquitinase Otub1 controls the activation of CD8(+) T cells and NK cells by regulating IL-15-mediated priming. Nat Immunol, 2019. 20(7): p. 879-889.

What is claimed is:

1. An ex vivo method for producing CD8 T cells and/or natural killer (NK) cells modified to express a reduced level of Otub1 compared to unmodified CD8 T cells and/or NK cells comprising:
(a) culturing a starting population of CD8 T cells and/or NK cells;
(b) introducing a vector that inhibits the expression of Otub1; and
(c) expanding the modified CD8 T cells and/or NK cells.

2. The method of claim 1, wherein the vector encodes an Otub1 inhibitory RNA.

3. The method of claim 1, wherein the vector encodes an shRNA that inhibits Otub1 mRNA expression.

4. The method of claim 1, wherein the vector encodes a construct to modify the Otub1 gene, thereby preventing Otub1 expression.

5. The method of any of claims 1-4, wherein the vector is a lentiviral vector or retroviral vector.

6. The method of any of claims 1-5, wherein introducing comprises transduction, transfection, or electroporation.

7. The method of any of claims 1-6, wherein the modified CD8 T cells and/or NK cells are further modified to express a CAR and/or a TCR.

8. The method of any of claims 1-7, wherein the starting population of CD8 T cells and/or NK cells is obtained from a sample of autologous tumor infiltrating lymphocytes having antitumor activity, cord blood, peripheral blood, bone marrow, CD34⁺ cells, or induced pluripotent stem cells (iPSCs).

9. The method of any of claims 1-8, wherein the population of modified CD8 T cells and/or NK cells are GMP-compliant.

10. A population of modified CD8 T cells and/or NK cells produced according to the methods of any one of claims 1-9.

11. A pharmaceutical composition comprising the population of modified CD8 T cells and/or NK cells of claim 10 and a pharmaceutically acceptable carrier.

12. A composition comprising an effective amount of the modified CD8 T cells and/or NK cells of claim 10 for use in the treatment of a cancer in a subject.

13. The use of a composition comprising an effective amount of the modified CD8 T cells and/or NK cells of claim 10 for the treatment of a cancer in a subject.

14. A method of treating a cancer in a patient comprising administering an anti-tumor effective amount of modified CD8 T cells and/or NK cells of claim 10 to the subject.

15. The method of claim 14, wherein the cancer is a solid cancer or a hematologic malignancy.

16. The method of claim 14, wherein the modified CD8 T cells and/or NK cells are autologous to the patient.

17. The method of claim 14, wherein the modified CD8 T cells and/or NK cells are derived from a sample of autologous tumor infiltrating lymphocytes having antitumor activity.

18. The method of claim 14, wherein the modified CD8 T cells and/or NK cells are allogeneic.

19. The method of claim 14, wherein the modified CD8 T cells and/or NK cells are HLA matched to the patient.

20. The method of claim 14, wherein the modified CD8 T cells express a CAR polypeptide and/or a TCR polypeptide.

21. The method of claim 20, wherein the modified CAR and/or TCR has antigenic specificity for CD19, CD319/CS1, ROR1, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

22. The method of claim 14, wherein the modified CD8 T cells and/or NK cells are administered to the subject intravenously, intraperitoneally, or intratumorally.

23. The method of any of claims 14-22, further comprising administering at least one additional therapeutic agent to the patient.

24. The method of claim 23, wherein the at least one additional therapeutic agent is selected from the group consisting of chemotherapy, radiotherapy, and immunotherapy.

25. The method of claim 24, wherein the at least one additional therapeutic agent is an immunotherapy.

26. The method of claim 25, wherein the immunotherapy is an immune checkpoint inhibitor.

27. The method of claim 26, wherein the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR).

28. The method of claim 27, wherein the immune checkpoint inhibitor inhibits PD-1 or CTLA-4.

29. The method of any one of claims 14-28, further comprising lymphodepletion of the subject prior to administration of the modified CD8 T cells and/or NK cells.

30. The method of claim 29, wherein lymphodepletion comprises administration of cyclophosphamide and/or fludarabine.

31. The method of any one of claims 14-30, wherein the method increases the frequency of CD8 effector T cells in the patient's cancer.

32. The method of any one of claims 14-30, wherein the method increases the frequency of stage 4 mature NK cells in the patient's cancer.

33. The method of any one of claims 14-30, wherein the method overcomes immune tolerance in the patient.

34. The method of any one of claims 14-30, wherein the method reduces CD8 T cell self-tolerance in the patient.

35. The method of any one of claims 14-30, wherein the method increases the number of tumor infiltrating CD8 T cells and NK cells in the patient's cancer.
